(19) United States
(12) Patent Application Publication
(10) Pub. No.: US 2002/0155443 A1
(43) Pub. Date: Oct. 24, 2002

(54) Metal binding DNA interactive compounds

(76) Inventors: Sean M. Kerwin, Round Rock, TX (US); Mark M. McPhee, Durham, NC (US)

Correspondence Address: ERIC B. MEYERTONS, CONLEY, ROSE & TAYON, P.C., P.O. BOX 398, AUSTIN, TX, 78767-0398, US

(21) Appl. No.: 09/771,016

(22) Filed: Jan. 25, 2001

Related U.S. Application Data
(60) Provisional application No. 60/178,082, filed on Jan. 25, 2000.

(51) Int. Cl. C12Q 1/68; C07H 21/04; C07K 9/00

(52) U.S. Cl. 435/6; 536/24.3; 530/322; 534/727

(57) ABSTRACT

In an embodiment, a novel DNA-interactive compound is formed by coupling an alkali metal ion binding moiety with a DNA interactive moiety. An alkali metal ion binding moiety is any group capable of binding alkali metal ions (e.g., lithium, sodium, potassium, etc.). The DNA-interactive moiety is a group of atoms or functionality capable of covalently modifying DNA, through, for example, alkylation, cleavage, metalation, hydrolysis, or crosslinking.

### PRIORITY CLAIM

This application claims priority to U.S. Provisional Application No. 60/178,082 entitled “Metal Binding DNA Interactive Compounds,” filed Jan. 25, 2000.

### BACKGROUND OF THE INVENTION

1. Field of the Invention

The compounds disclosed herein generally relate to compounds capable of interacting with both DNA and metal ions. More particularly, the compounds disclosed herein are composed of a metal binding moiety coupled to a DNA interactive moiety.

2. Description of the Related Art

Treatment of cancer requires discrimination between populations of human cells. However, as few as five or six critical genomic mutations may ablate the normally strict control of the cell cycle and lead to transformation of a healthy cell into a cancerous one.^{1.1} The inherent difficulty associated with cancer chemotherapy is that the biological composition of cancer cells is quite similar to that of healthy cells. Indeed, the hallmark of most cancer cells, i.e., unregulated cell division, is a result of insufficient checks on otherwise normal cellular processes. Without a molecular target which is uniquely critical to the viability of cancer cells, ammunition launched to destroy cancer cells will undoubtedly harm healthy cells as well.

Most of the clinically useful anticancer drugs used today are antiproliferative agents.^{1.2} The effectiveness of these compounds is often curtailed by dose-limiting toxicity, which prevents their administration in larger doses. In general, the toxicological side-effects due to these agents are the result of indiscriminate action at healthy, proliferating tissues as well as cancerous tissues.^{1.3} This is a direct consequence of the biological similarity of the cytotoxin target in both normal and cancerous cells. Therefore, a large effort is being expended toward the discovery of molecular entities or cellular processes that distinguish cells as cancerous.

A biological characteristic of many tumors is rapid proliferation. Thus, early anticancer efforts focused on the development of antiproliferative agents. While the macromolecular targets of antiproliferative agents as a class varies widely, a common theme to their mode of action is disruption of DNA function. Depending on the agent, these targets may occur at the level of nucleobase synthesis, DNA synthesis or replication, or transcription of genetic information to mRNA. The usefulness of many of these agents is hampered by toxicity at rapidly proliferating, healthy tissues such as gut epithelia and bone marrow.^{1.4} Furthermore, as many solid tumors grow slowly, these tissues may be somewhat refractory to antiproliferative treatment. Finally, some cancerous tissues are known to overexpress proteins which mediate the expulsion of anticancer agents. This mechanism of chemotherapy resistance has additionally hindered (sometimes severely) the usefulness of existing agents.^{1.5}

The last two decades have witnessed the discovery of natural products (e.g., the enediyne antibiotics and the CC-1065/duocarmycin family) which display exceedingly potent antitumor action derived from modification of DNA.^{1.6} While useful as leads, these agents typically have limited clinical utility due to their lack of selectivity for modifying cancer-cell DNA. The development of significantly improved anticancer agents derived from these natural products hinges on strategies for ensuring selective action at tumor tissue loci.

Many advances in the understanding of cell cycle regulation, tumor cell biology, and the transformation of normal cells into tumor cells have been made since the development of the antiproliferative agents.^{1.7} Cellular events and molecular entities have been discovered which distinguish cancer cells from normal cells. These findings have shaped current anticancer drug design.

One strategy which has been proposed to increase the effectiveness of anticancer agents seeks to increase the concentration of a cytotoxin within the region surrounding or inside a cancer cell. For example, antibodies have been developed which bind to unique protein epitopes on tumor cell surfaces. These antibodies can be conjugated to enzymes capable of activating cytotoxic prodrugs.^{1.8} In this manner, an inert compound, upon interaction with such a cancer cell-bound conjugate, is converted to a cytotoxin preferentially near its tumor cell target. In a similar vein, certain classes of molecules (e.g., porphyrins^{1.9}) have demonstrated preferential concentration within tumor cells via active transport processes. These agents can be endowed with appropriate functionality such that an externally added stimulus converts the cancer-cell concentrated agent into an active anticancer compound. Such species have proven to be effective cancer cell-selective cytotoxins in photodynamic drug therapy.^{1.10}

Another anticancer drug strategy that is being actively investigated is the prodrug approach.^{1.11} Here, a physical or chemical property that sufficiently distinguishes the tumor cell is exploited to chemically convert a prodrug into a cytotoxin. Some of these cancer cell-distinguishing activation processes that have been investigated include low extracellular pH^{1.12}, decreased oxygen tension^{1.13} and overexpression of enzymes.^{1.14} A potential advantage of the prodrug activation approach over standard antiproliferative therapeutics is that slowly growing solid tumors may be targeted as well as those that are rapidly proliferating.^{1.4}

The vast majority of anticancer agents exert their action by disrupting DNA function. DNA is a logical target for antiproliferative agents because DNA replication must necessarily precede cell division.^{1.15} There are many potential sites of interaction for drugs which disrupt DNA function. For example, the widely-used compound 5-fluorouracil blocks the synthesis of thymidine via inhibition of thymidylate synthase.^{1.16} This results in a cessation of DNA synthesis by inhibiting the synthesis of one of the DNA building blocks. Agents which covalently crosslink DNA, such as mitomycin C^{1.17} or the nitrosoureas^{1.18}, are believed to disrupt DNA replication and/or DNA transcription. There is increasing evidence that some intrastrand DNA crosslinking agents (e.g., cisplatin) may produce a bend in the DNA which is recognized by DNA binding proteins.^{1.19} The DNA-bound protein may prolong the lifetime of the DNA adduct by preventing recognition of the modified DNA by repair enzymes.

A substantial scientific effort has been undertaken to understand how unique sequences of DNA can be specifically recognized.^{1.20} As the roles of various proteins in maintaining the tumor cell cycle become further delineated, the DNA sequences that code for these proteins have attracted interest as viable anticancer targets. Along these lines, some groups have developed a molecular toolbox containing monomeric subunits capable of recognizing the minor groove of any DNA base pair.^{1.21} Using the target DNA sequence as a blueprint, a linear polymer (polyamide) may be fashioned from the appropriate subunits. Some of these polyamides have demonstrated impressive DNA sequence selectivity.^{1.22}

Recently, DNA secondary structure has been investigated as a target for anticancer drug design. Guanine-rich regions of single-stranded DNA found at the ends of chromosomes (telomeres) may form an intramolecular hairpin structure (G-quadruplex) under certain conditions.^{1.23} G-quadruplex-binding compounds are thought to stabilize these structures and prevent the DNA from serving as a template for the DNA-polymerizing enzyme telomerase.^{1.24} The resulting telomerase inhibition may, after several rounds of DNA replication, critically (and perhaps lethally) shorten the abbreviated cancer cell telomeres.

The production of DNA lesions can be a lethal cellular event.^{1.7} As a result, molecules which demonstrate a potential for cleaving DNA are being intensively studied for use as anticancer agents. Indeed, some of the most potent antitumor agents known are DNA-cleaving agents.^{1.25} Additionally, certain DNA-cleaving agents possess the capacity to induce apoptosis, or programmed cell death, in tumor cells.^{1.7}

DNA-cleaving agents are a structurally diverse class of compounds. While some of the members of this class of agents are chemically complex natural products^{1.26}, others are fairly simple transition metal ion-binders²⁷ or hydroxylated aromatic compounds.^{1.28} Despite the structural variety within the group, molecules which cleave DNA do so by a few general mechanisms. In most cases, DNA cleavage is the result of radical-induced oxidative damage, either to the nucleobase or the deoxyribose moiety.^{1.29} The reactive radical species can either be oxygen-centered or carbon-centered. A mechanism encountered less frequently under physiological circumstances is DNA cleavage occurring as a result of phosphate backbone^{1.30} or nucleobase alkylation.^{1.31} DNA cleavage can also result from inhibition of topoisomerases.^{1.32}

The enediyne antibiotics are a group of natural products which have attracted considerable interest in the biomedical community because of the phenomenal cytotoxicity some of the members have demonstrated against tumor cells.^{1.33} The cytotoxic potency of the enediyne antibiotics is widely believed to be a result of efficient double-stranded DNA cleavage.^{1.34} Most of the agents in this class of natural products require a chemical activation event^{1.35} to allow the enediyne moiety within the agent to undergo a Bergman cycloaromatization reaction.^{1.36} This results in the production of a highly reactive carbon-centered diradical structure known as a 1,4-diyl. This highly energetic diradical species can damage both strands of DNA via deoxyribose hydrogen atom abstraction.^{1.37} The enediyne antibiotics are, therefore, dissimilar from the vast majority of radical-based DNA-cleaving agents, both synthetic and naturally occurring, which damage DNA via the production of oxygen-centered radicals (e.g., hydroxyl radical).^{1.29}

Another class of DNA-cleaving agents are the propargylic sulfones.^{1.40} Under mildly basic conditions, the propargylic sulfone moiety isomerizes to an allenylic sulfone. This allene is an efficient Michael acceptor which can alkylate DNA.^{1.41} The ability of propargylic sulfones to produce frank DNA stand scission in vitro following alkylation is rather uncommon among DNA alkylators. Generally, DNA lesions due to alkylation require treatment with base (e.g., piperidine) and heat after the initial alkylation event in order to effect strand scission.^{1.31} The increased lability of the deoxyribose-base glycosidic linkage in the propargylic sulfone-DNA adduct may be due to the greater electron withdrawing effect of the alkenyl group attached to the nucleobase nucleophile. While several groups have demonstrated that propargylic sulfone-containing molecules inhibit the growth of cancer cells, the correlation of this event with DNA cleavage remains to be established.^{1.44}

As mentioned previously, the effectiveness of many cytotoxic agents is compromised by a lack of selectivity for killing tumor cells. There is evidence that the intracellular levels of some metal ions are different for tumor cells versus normal cells. As such, these differences might conceivably be exploited toward the design of metallo-regulated agents which selectively disrupt cancer cell function. Elevation and altered ratios of the intracellular levels of several species of metal ions, including sodium, potassium, calcium, iron, zinc and cadmium, during pre-neoplastic events, proliferative events, and tumor cell growth and metastasis suggests a role for these ions in these processes.^{1.52} For example, there is evidence indicates that intracellular sodium ion concentration is greater within tumor cells versus normal cells. Thus, the development of sodium ion-regulated cytotoxins may provide agents which selectively destroy tumor cells.

### SUMMARY OF THE INVENTION

In an embodiment, a novel DNA-interactive compound is formed by coupling an alkali metal ion binding moiety with a DNA interactive moiety. An alkali metal ion binding moiety is any group capable of binding alkali metal ions (e.g., lithium, sodium, potassium, etc.). Such moieties include heteroatom-containing groups, such as ethers, amines, esters, and amides. Without limiting the scope of the compounds envisioned, specific examples of these alkali metal ion binding moieties include crown ethers, cryptands, sepulchrates, spherands, calixaranes, cyclens, monensin or other polyether antibiotics, valinomycin, enniatin-B, or other cyclic peptide antibiotics, or podands.

The DNA-interactive moiety is a group of atoms or functionality capable of covalently modifying DNA, through, for example, alkylation, cleavage, metalation, hydrolysis, or crosslinking. Without limiting the scope of the compounds envisioned, specific examples of these DNA-interactive moieties include propargylic sulfones, enediynes or aza-enediynes, eneynallenes or aza-eneynallenes, cyclopropylpyrroloindoles (CPIs) or CPI analogs, pyrrolobenzodiazepines, nitrogen mustards, sulfur mustards, epoxides, aziridines, nitroso compounds, iron-EDTA complexes and analogs, sulfonate esters, alkyl halides, ortho-quinone-generating moieties, photo-activated DNA cleavage agents such as nitro compounds, azides, benzophenones, quinobenzoxazines, fluoroquinolones, Rh-complexes, Ru-complexes, Cu-complexes, Co-complexes, bleomycin or bleomycin analogs, porphyrins and porphyrin analogs, and metal salen complexes.

In one embodiment, the alkali ion binding DNA interactive compound is a compound of general structure I:

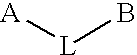

Where A is an alkali metal ion binding moiety, B is DNA-interactive moiety capable of covalent modification of DNA, and L is a linking group. Examples of linking groups include, but are not limited to alkyl, alkenyl, alkynyl, phenyl, phenylalkyl, arylalkyl, aryl, carbocyclic ring, an amine, a sulfide, an ether, a ketone, ester, amide, or imine.

In another embodiment, the alkali ion binding DNA interactive compound is a compound of general structure general structure II:

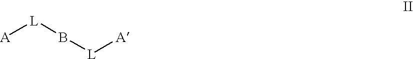

Where A and A′ are the same or different alkali metal ion binding moiety, B is DNA-interactive moiety capable of covalent modification of DNA, and L and L′ are linking groups.

In another embodiment, the alkali ion binding DNA interactive compound is a compound of general structure III:

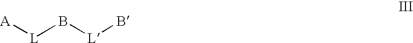

Where A is an alkali metal ion binding moiety, B and B′ are DNA-interactive moieties capable of covalent modification of DNA, and L and L′ are linking groups.

In another embodiment, the alkali ion binding DNA interactive compound is a compound of general structure IV.

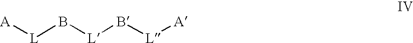

Where A and A′ are the same or different alkali metal ion binding moiety, B and B′ are DNA-interactive moieties capable of covalent modification of DNA, and L, L′, and L″ are linking groups.

In another embodiment, the alkali ion binding DNA interactive compound is a compound of general structure V:

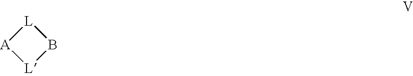

Where A is an alkali metal ion binding moiety, B is a DNA-interactive moiety capable of covalent modification of DNA, and Land L′ are linking groups.

In another embodiment, the alkali ion binding DNA interactive compound is a compound of general structure VI:

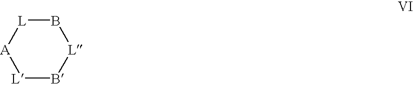

Where A is a n alkali metal ion binding moiety, B and B′ are DNA-interactive moieties capable of covalent modification of DNA, and L and L′ are linking groups.

In another embodiment, the alkali ion binding DNA interactive compound is a compound of general structure VII:

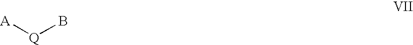

Where A is an alkali metal ion binding group and B is a leaving group and Q is a bond or group that is cleaved upon metal ion binding by A such that when Q is cleaved A, B, or both are DNA interactive agents capable of covalently modifying DNA.

In one embodiment, a series of bis(propargylic) sulfone crown ethers may be used as metallo-regulated DNA-cleaving agents. General methodology for the synthesis of bis(propargylic) sulfone crown ethers are described. Bis(propargylic) sulfone crown ethers may be produced in five steps from the corresponding ethylene glycol. The average of the overall yields for the series was 18%. Results of metal ion binding studies revealed that bis(propargylic) sulfone crown ethers showed affinity for a variety of metal ions. DNA cleavage assays of the bis(propargylic) sulfone crown ethers indicated that cleavage of DNA occurs in the presence of a variety of metal cations. Thus, the inclusion of a DNA-cleaving propargylic sulfone within metal ion-binding crown ethers does indeed produce agents which cleave DNA in a manner that is dependent upon the nature of the primary alkali metal ion present in the assay.

In another embodiment, a series of 15-crown-5-containing propargylic sulfones may be used as metal binding DNA interactive compounds. These compounds may be prepared by the esterification of chloromethyl benzoic acid analogues with a crown ether alcohol. The esterified products may be converted into isothiuronium salts which are decomposed to thiol derivatives. The thiol deriviatives may be alkylated to form sulfides. Finally the sulfides may be oxidized to form the 15-crown-5 propargylic sulfones.

In another embodiment, enediyne-crown ether may be used as metal binding DNA interactive compounds. In one embodiment, the enediyne crown ethers may be synthesized via a multistep procedure in which a macrocyclization step sets the stage for enediyne elaboration. The final step in this route involved a cheletropic ring contraction (Ramberg-Bäcklund reaction) to install the alkene portion of the enediyne. Alternatively, the enediyne crown ether may be synthesized via a carbenoid coupling strategy in which macrocyclization and enediyne formation are concurrent.

In another embodiment, the enediyne podands may be synthesized via a copper(I) and palladium(O)-catalyzed cross-coupling (Castro-Stephens reaction) between a propargyl ether and cis-ethylene dichloride.

In another embodiment, biphenyl enediyne crown ethers may be used as a metal binding DNA interactive compound. These compounds may be formed by sequentially forming a crown ether and an enediyne about a biphenyl scaffold. These compounds were found to be potent DNA-cleaving agents.

### DETAILED DESCRIPTION OF THE PREFERRED EMBODIMENTS

A current strategy in anticancer drug design involves exploiting tumor-distinguishing phenomena for the selective localization or activation of cytotoxic agents. Destruction of DNA has been demonstrated to be an extremely efficient means by which cytotoxins kill tumor cells. The proliferative, metastatic, and growth capabilities of many tumor cells appear to require either an influx or sustained elevation of intracellular sodium ions. To the best of our knowledge, this potential cancer cell-discriminating phenomenon has not been exploited as a means of selectively targeting tumor cells for destruction.

In one embodiment, crown ethers may be used as an alkali metal recognition moiety coupled to a DNA interactive moiety. The crown ether may be coupled with known DNA-cleaving moieties to create a metal binding DNA interactive compound. In some embodiments, either of two DNA-cleaving structures may be coupled with the crown either to produce the sodium binding DNA interactive compound: the electrophile-precursor propargylic sulfone and the radical-producing enediyne.

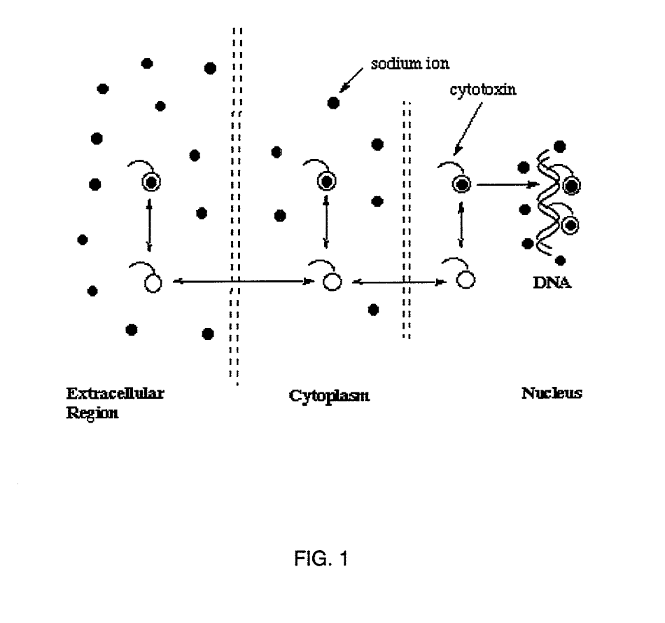

Depicted in FIG. 1 is a scheme of how it is believed that sodium binding DNA interactive compounds cleave DNA in a metallo-regulated manner. The agents would likely exist in equilibrium as metal ion-complexed and uncomplexed forms. Additionally, the uncomplexed form of the agents is believed to partition into the cell and into cellular compartments such as the nucleus. Once inside the nucleus, sodium binding by the metal ion-recognition unit of the cytotoxin may confer the resulting complex with a positive charge and consequently greatly increase the affinity of the cytotoxin for the anionic DNA target. The formation of cationic species from prodrugs has been shown to be an effective mechanism for targeting cytotoxins to DNA.⁶⁶ It is believed that the higher intranuclear sodium ion concentration that is found within many tumor cells will result in a greater localization of sodium ion-complexed DNA-cleaving agents near the DNA target within these cells versus normal cells. As the concentration of extracellular sodium is much higher to that found intracellularly, the risk exists that in vivo a sodium ion-complexing agent will exists primarily extracellularly as a cationic species. This issue might be successfully addressed by utilizing metal ion-binding elements whose complex stability constants for sodium in water allow for a satisfactory fractions of host molecules to remain uncomplexed and thus capable of penetrating the cellular membrane.

An additional mechanism of effecting sodium ion-regulated DNA destruction other than via tumor cell-selective localization can be imagined with enediyne-containing crown ethers cytotoxins. The cytotoxic capacity of most of the naturally-occurring enediynes is realized only after the molecules experience a triggering event. In another embodiment, an enediyne-containing crown ether utilizes sodium ion recognition as the triggering event to unleash the DNA-cleaving potential of the agent.

The mitochondrial membrane potential in tumor cells is reported to be elevated versus that for normal cells.^{1.67} As such, lipophillic cationic dyes^{1.68} and metal ion complexes^{1.69} have been observed to concentrate within these organelles in cancer cells. Thus, we may reasonably assume that the sodium ion-binding DNA-cleaving agents proposed in this work may effect an additional means of selective action against tumor cells due to this localization mechanism.

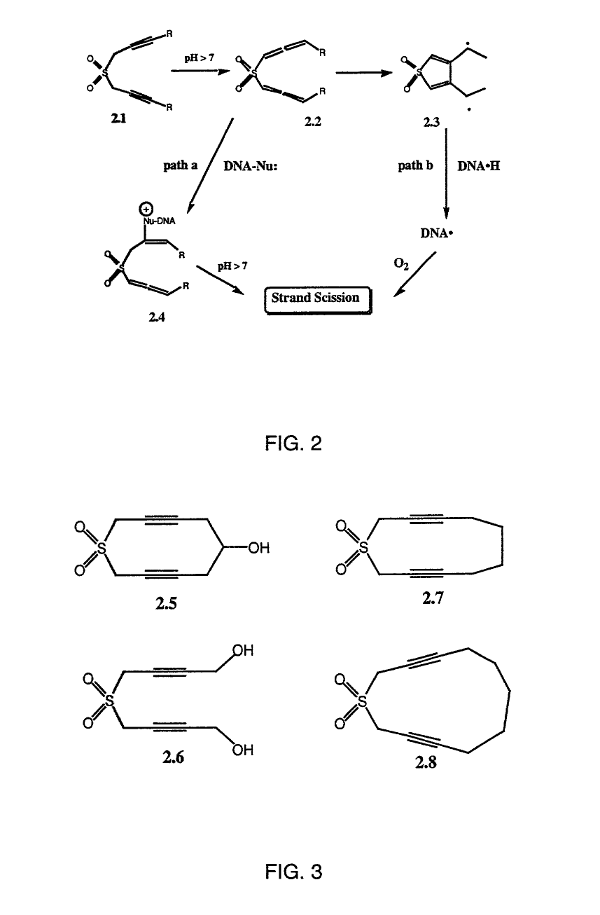

The DNA-cleaving properties of bis(propargylic) sulfones 2.1 (See FIG. 2) are known.^{2.1} These agents were designed with the capacity to enter two manifolds in which two putative DNA-cleaving intermediates could be generated (see FIG. 2). In both paths, isomerization to a bis(allenic) species, 2.2, is required. This reactive intermediate may then act as an electrophile to alkylate DNA (path a) or perhaps undergo a Braverman-Duar reaction^{2.2} (path b) to produce diradical species 2.3. While alkylated DNA species 2.4 may be expected to undergo depurination and subsequent strand scission at pH>7^{2.3}, the π,π-type diradical 2.3 may be energetic enough to abstract hydrogen atoms from the sugar backbone of DNA in order to effect strand scission.^{2.1}

Examples of DNA-cleaving bis(propargylic) sulfones are depicted in FIG. 3. DNA cleavage studies revealed that the cyclic bis(propargylic) sulfones displayed an interesting relationship between ring size and potency for damaging DNA. Compound 2.7 was found to be more potent than compound 2.8 and much more potent than compound 2.5 and the acyclic species 2.6. While somewhat less potent than compound 2.8, compound 2.5 was equipotent with the closely related acyclic compound 2.6.

In one embodiment, a metal binding DNA interactive molecule is a bis(propargylic) sulfone crown ether, 2.12. This compound was designed to act as a metallo-regulated DNA-cleaving agent.

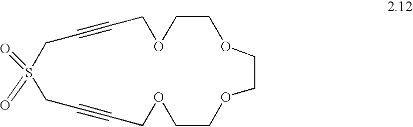

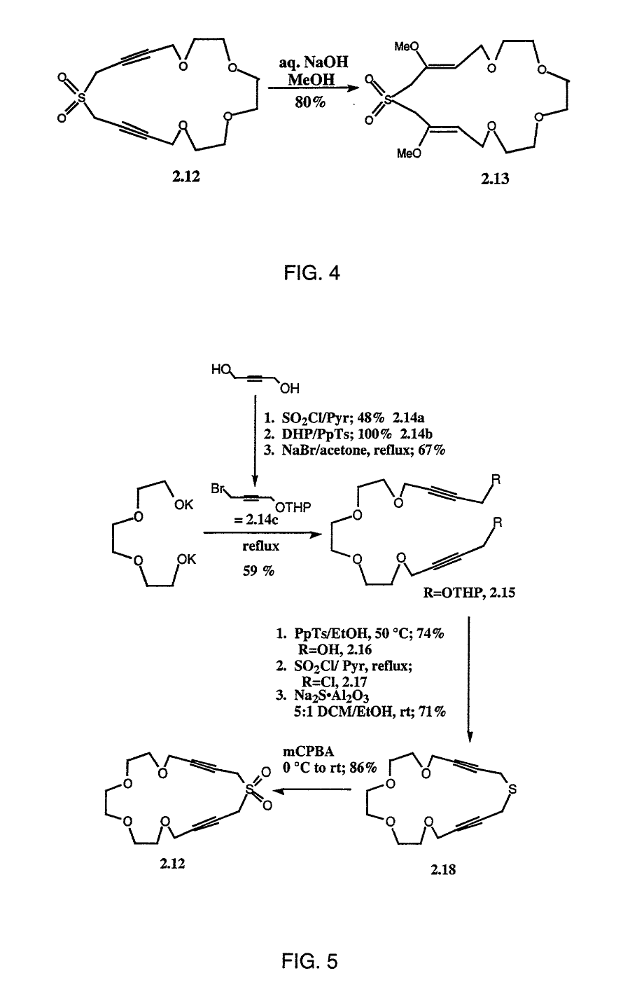

The molecule possesses a metal ion recognition unit (polyether) and two propargylic sulfone moieties, each of which may effect DNA damage via the alkylation mechanism depicted in FIG. 2. Compound 2.12 exhibited a slight affinity for alkali metal ions, as determined by picrate extraction analysis, and demonstrated dose-dependent cleavage of Form I DNA under mildly alkaline (pH 8.5) conditions. It is believed that the DNA damage due to 2.12 is insensitive to either the addition of radical scavengers or the exclusion of oxygen. Additionally, treatment of a methanolic solution of 2.12 with aqueous NaOH led to the production of the bis(enol ether) 2.13 as depicted in FIG. 4. Taken together, these results favor an alkylation mechanism (FIG. 4, path a) for the damage of DNA by bis(propargylic) sulfone crown ether 2.12.

One embodiment of the synthesis of compound 2.12 is shown in FIG. 5.^{2.5} The potassium bis(alkoxide) of triethylene glycol may be alkylated with THP-protected 4-bromo-2-butynol, 2.14. Bromide 2.14 may be prepared in three steps from 2-butyn-1,4-diol. Alcohol deprotection and subsequent chlorination may be used to afford dichloride 2.17. Macrocyclization to sulfide 2.18 may be accomplished in good yield under heterogeneous conditions with Na₂S-impregnated alumina.^{2.9} Finally, oxidation to the sulfone may be accomplished with mCPBA.

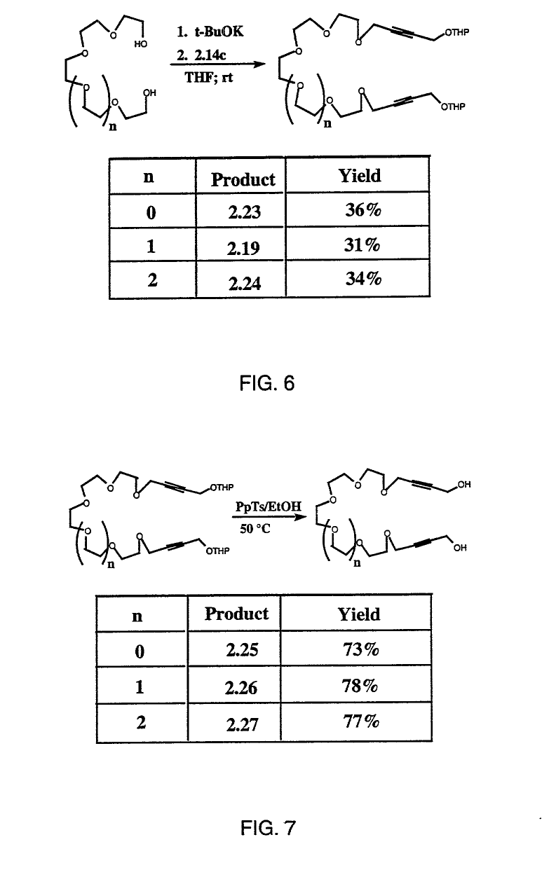

The methodology depicted in FIG. 5 may also be used to synthesize other bis(propargylic) sulfone crown ethers. FIG. 6 depicts an embodiment of a synthesis of synthetic precursors to homologues of bis(propargylic) sulfone crown ethers, In one embodiment, the bisalkylated products, depicted in FIG. 6, may be synthesized by treatment of the diol with a base (e.g., t-BuOK) and quick addition of an alkylating agent (e.g., bromide 2.14c) at room temperature.

The tetrahydropyran ethers of the alkylated crown ethers are removed to allow access to the hydroxyl functionality. This deprotection may be achieved in good yield with PpTs in EtOH at 55° C. In some instances the produced diols may be somewhat thermally labile. In some embodiments mild base (e.g., aqueous bicarbonate) is used to wash the crude diols during workup to minimize decomposition of the products. FIG. 7 summarizes the results of these deprotections.

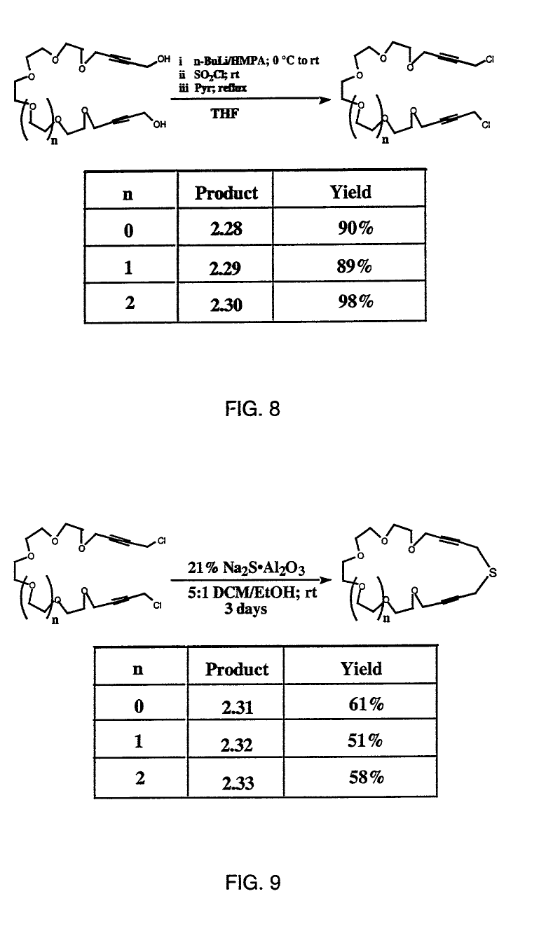

In one embodiment, excellent yields of dichlorinated material may be obtained by reacting a mixture of the dialkoxide, resulting from treatment of the diol with a base (e.g., n-BuLi/HMPA), with a large excess of thionyl chloride and decomposing the resulting bis(chlorosulfite) to the dichloride by heating the reaction mixture under reflux overnight in the presence of pyridine. In some instances, the use of a basic (e.g., aqueous bicarbonate) wash of the crude reaction mixture during workup improved the yileds of the dichlorinated material. In another embodiment, TMEDA may be used in place of HMPA, with similar results. The results of the chlorinations of the ethylene glycol homologs are summarized in FIG. 8.

The cyclization of the dichloride to a sulfide may be accomplished by the use of Na₂S-impregnated alumina. In some embodiments, lower ratios of Na₂S to alumina (21% w/w Na₂S) in the impregnated reagent^{2.9}, and more dilute reaction conditions (15 mM) allows for the formation of the desired macrocyclic sulfides in moderate yields while minimizing polymer formation. The results of the macrocyclizations are summarized in FIG. 9.

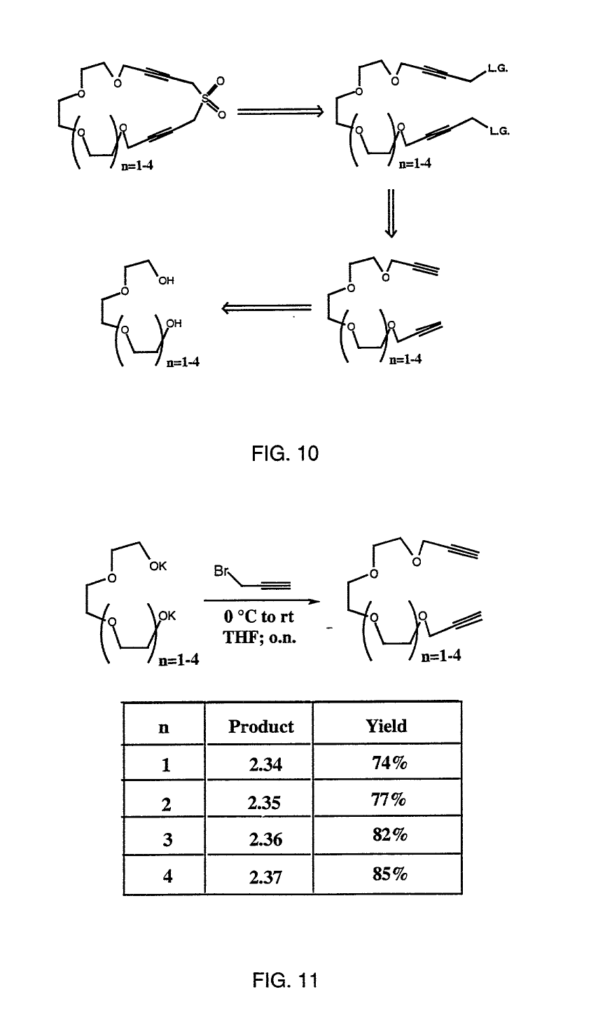

An alternate route to the synthesis of bis(propargylic) sulfone crown ethers is depicted in FIG. 10. The cogent features of the revised route are the use of propargyl bromide to alkylate the glycols, homologation of the incipient bis(propargyl ether), and installation of an alumina-stable functional group to ensure success of the macrocyclization reaction.

The alternate route begins with the alkylation of an ethylene glycol with propargyl bromide. This addition may be accomplished by inverse addition of the bis(alkoxide) to a well-stirred, ice-water bath-cooled solution of propargyl bromide. In this manner, the alkylation of ethylene glycols in high yield may be accomplished; some examples are summarized in FIG. 11.

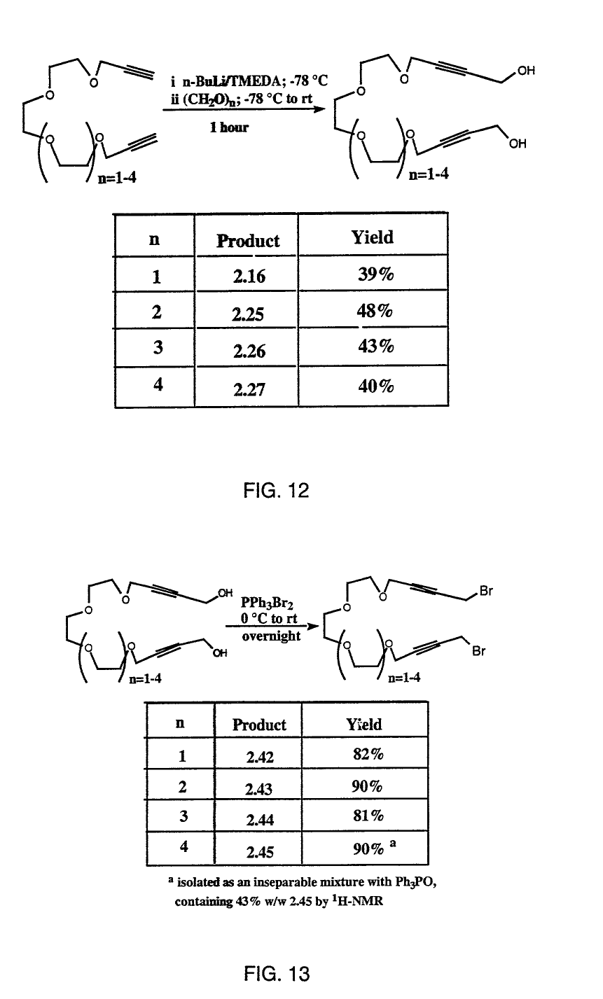

After the ethylene glycols are reacted with propargyl bromide, the terminal acetylene may be hydroxymethylated with formaldehyde in the presence of a strong base to form propargyl alcohols. In one embodiment, the base used is an alkyl lithium base, e.g., n-butyl lithium. The hydroxymethylation reaction may be controlled by the following reaction conditions. First, the use of co-solvents, such as TMEDA, inhibits aggregation of the bis(acetylide) as the reaction temperature warms to near 0° C. When TMEDA was used in these reactions, the bis(acetylide) was observed to form a flocculent suspension in intimate contact with the solid paraformaldehyde reagent as the reaction temperature warmed to near 0° C. The resulting increase in reacting surface area may explain the increased yields observed when a co-solvent was employed. Second, improved yields may be accomplished by the use of lower reaction temperatures. Third, the nascent diol product, is, in some instances, sensitive to acid. Therefore, improved yields may be obtained when saturated solutions of NaH₂PO₄ were used in place of NH₄Cl to quench the reaction. A homologous series of diols may be produced by this general method. Some of the diols produced by this method are depicted in FIG. 12.

In one embodiment, the diols may be converted into dibromides via the use of in situ generated PPh₃Br₂. After the reaction of the diols with the PPh₃Br₂ is completed, the dibromides may be purified using silica gel chromatography. Alternatively, the reaction products may be used in subsequent steps without further purification. A homologous series of dibromides produced using this method is shown in FIG. 13.

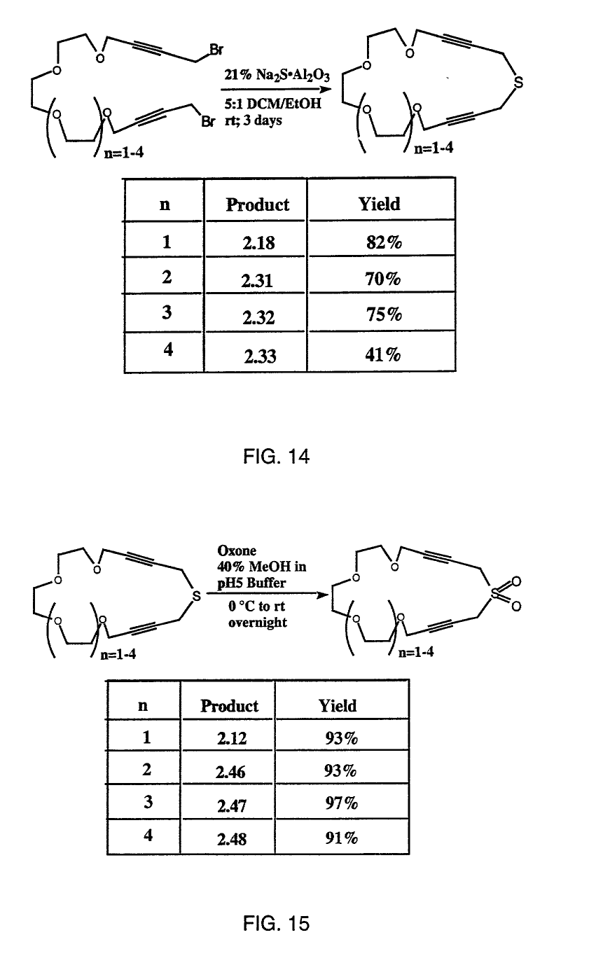

The resulting dibromides may be converted to a bis(propargylic) sulfide crown ether by the reaction of the dibromide with Na₂S-impregnated alumina. FIG. 14 depicts results of some exemplary macrocyclization reactions.

The bis(propargylic) sulfide crown ether may be oxidized with a suitable oxidation agent to form a bis(propargylic) sulfone crown ether. Suitable oxidation agents include peracetic acid, mCPBA, and OXONE. The use of OXONE allows the use of a buffered reaction mixture in which destructive acids produced during the reaction may be consumed. Excess oxidant may be extracted from the crude reaction mixture with water during workup. This alleviates the need for neutralization and extraction with allene-forming alkaline solutions during workup. This protocol affords excellent yields of the desired bis(propargylic) sulfone crown ethers, which, in most cases, were analytically pure after workup. The results of some oxidations with OXONE are depicted in FIG. 15.

After the bis(propargylic) sulfone crown ethers were synthesized, the metal binding ability of the compounds may be assessed. Many methods are available for assessing the stability constants of a ligand-metal ion complex in aqueous systems. In general, a measurement is obtained, for example by spectrophotometric, spectroscopic or potentiometric means, of the concentration of at least one component in the equilibrium mixture. Knowledge of the stoichiometry of the ligand-ion system allows for calculation of the concentrations of the other species in solution and hence the equilibrium constant for the binding reaction.^{2.12} For ligands that exhibit limiting solubility in water, mixed solvent systems, such as dioxane-water or MeOH-water, may be used, and the stability constant thereby derived is particular to that solvent system.

In one embodiment, the metal extraction ability of the bis(propargylic) sulfone crown ethers may be studied using a picrate extraction technique. A picrate extraction technique is a spectroscopic method for assessing the formation constants of hosts, H, for metal ions, M⁺, in an organic solvent.^{2.14} Briefly, an aqueous solution of an alkali metal picrate is mixed with a solution of host dissolved in an organic solvent, e.g. CHCl₃, and the layers are allowed to separate. The quantity of colored, anionic picrate that the host has extracted via formation of a lipophillic ion pair is determined by spectrophotometrically measuring an aliquot (suitably diluted with MeCN) of the organic layer. From the A₃₈₀ value and Beer's Law, A=εbc, one can obtain the concentration of the host-metal picrate complex in the organic layer. Cram and co-workers have determined the extinction coefficient, ε, for lithium, sodium and potassium picrate in MeCN at 380 nm (25° C.) to be 16,900 M⁻¹ cm⁻¹.^{2.13} Thus, one can calculate the extraction coefficient, Kₑ, according to equation 2.3

$${K}_{e} = \frac{{[ {M}^{+} \cdot H \cdot {Pic}^{-} ]}_{CHCl 3}}{( {[ {M}^{+} ]}_{H 2 O} ) ( {[ {Pic}^{-} ]}_{H 2 O} ) ( {[ H ]}_{CHCl 3} )} \qquad ( 2.3 )$$

The determined value of Kₑ, together with the value of the distribution coefficient, K_{d}, for an alkali metal ion picrate partitioning between water and chloroform (see equation 2.4), allows one to calculate the metal-host complex association constant, Kₐ, according to equation 2.5.^{2.13} Cram and co-workers have determined K_{d} values for lithium, sodium and potassium picrate to be 1.42 E⁻³ M⁻¹, 1.74 E⁻³ M⁻¹, and 2.55 E⁻³ M⁻¹, respectively.^{2.16}

$$\begin{matrix}{K}_{d} = \frac{{[ {M}^{+} {Pic}^{-} ]}_{CHCl 3}}{( {[ {M}^{+} ]}_{H 2 O} ) ( {[ {Pic}^{-} ]}_{H 2 O} )} & ( 2.4 ) \\ {K}_{a} = \frac{{[ {M}^{+} \cdot H \cdot {Pic}^{-} ]}_{CHCl 3}}{( {[ {M}^{+} {Pic}^{-} ]}_{CHCl 3} ) ( {[ H ]}_{CHCl 3} )} \\ = {K}_{e} / {K}_{d} & ( 2.5 )\end{matrix}$$

This analysis assumes that the host, H, partitions minimally into the aqueous phase.

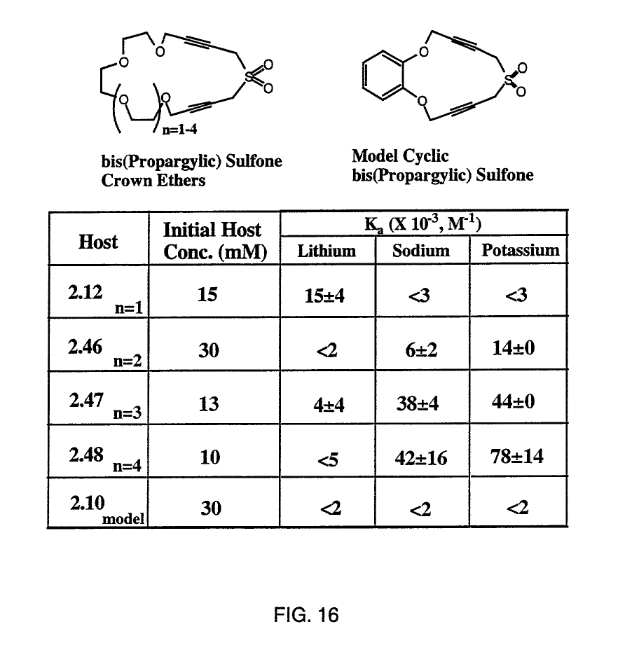

At least two separate determinations were made for each metal ion studied for bis(propargylic) sulfone crown ethers 2.12, 2.46, 2.47 and 2.48 and the model system 2.10. The complex association constants, Kₐ, for the metal-host complexes were determined as described above and the average value of these determinations are presented in FIG. 16.

The values reported in FIG. 16 represent the average and one standard deviation from at least two separate determinations. For hosts which did not display measurable affinity for a particular metal ion, the lower limit of the Kₐ value that could be determined is presented. Inspection of FIG. 16 reveals that the smallest homologue of the crown ether series, sulfone 2.12, displayed significant affinity for lithium ions. The next largest member of the crown ether series, sulfone 2.46, bound potassium ions with greater affinity than sodium ions. The hexaoxa-containing crown ether host, sulfone 2.47, bound potassium ions slightly better than sodium ions while exhibiting a slight affinity for lithium ions. The largest homologue of the crown ether series, sulfone 2.48, bound potassium with greater affinity than sodium ions. Not surprisingly, the model cyclic bis(propargylic) sulfone, compound 2.10), did not display measurable affinity for lithium, sodium or potassium ions.

The DNA cleaving capability of metal ion binding DNA interactive compounds may be investigated using a DNA cleavage assay. In one embodiment, Form I DNA from DH5αE. Coli that has been transfected with the plasmid pGAD424 may be used to assess the DNA cleavage capabilities of these compounds. From grown cultures of E. Coli, the plasmid DNA may be isolated using a QIAprep Miniprep kit (QIAGEN Inc.). The washed, plasmid DNA isolated with the kit may be resuspended in water to afford a stock solution of concentrated DNA. These solutions exhibit a satisfactory percentage (>75%) of Form I DNA. The stock solutions may be then diluted with sterile, pH 7.4 lithium, sodium or potassium phosphate buffers (containing alkali metal ions at a concentration of 20 mM) to afford DNA solutions that contained primarily a single alkali metal cation species. These alkali metal ion-enriched DNA preparations are herein referred to as M.DNAs (e.g., Li.DNA, Na.DNA, K.DNA).

A number of methods may be used to quantitate the cleavage fragments (Form II, relaxed circular; Form III, linear) produced by the reaction of the metal ion binding DNA interactive compounds with From I DNA. In one embodiment, laser scanning densitometry may be used.⁵ This technique uses a laser to scan the negative of a photograph of the ethidium bromide-stained, electrophoretically-separated DNA contained within an agarose gel. The density of each band or species of DNA from a given incubation roughly correlates with the quantity of that species.

Alternatively, a Molecular Dynamics Fluoroimager may be used to scan the ethidium bromide-stained agarose gels of the reaction products of the metal ion binding DNA interactive compounds. With this instrument, the fluorescence of very small regions of the entire gel may be digitally compiled. The integrated volume of each band may be calculated with the imaging software ImageQuant. To further enhance the extent of cleavage and thus aid in the distinction of cleavage efficiency between agents, the reaction samples may be briefly (90s) heated to 70° C.

With the data from the fluorescence-imaged gel, the degree of DNA cleavage due to the cleaving agent may be calculated. The following set of equations and assumptions may be used to establish the degree of DNA cleavage. Equation 2.6 describes the calculation of the percent cleavage of DNA due to the cleaving agent (the subscript “s” refers to reactions that contained a DNA-cleaving sample).^{2.5} The quantity of the linear fragment (Form III) is doubled since at least two cleavage events are necessary to produce this species from Form I DNA.

$$\% {cleavage}_{s} = \frac{2 \times Form {III}_{s} + Form {II}_{s}}{2 \times Form {III}_{s} + Form {II}_{s} + Form {I}_{s}} \qquad ( 2.6 )$$

As the conditions of the cleavage assay effected the cleavage of control samples that lacked bis(propargylic) sulfone, the quantity of this cleavage may be similarly calculated (see equation 2.7; the subscript “c” denotes control reactions).

$$\% {cleavage}_{c} = \frac{2 \times Form {III}_{c} + Form {II}_{c}}{2 \times Form {III}_{c} + Form {II}_{c} + Form {I}_{c}} \qquad ( 2.7 )$$

Finally, a normalized percent cleavage of DNA by the cleaving agent may be calculated by subtracting the per cent cleavage due to the control and dividing by the theoretical maximum amount of remaining cleavable DNA (see equation 2.8).

$$normalized \% {cleavage}_{s} = \frac{\% {cleavage}_{s} - \% {cleavage}_{c}}{100 - \% {cleavage}_{c}} \qquad ( 2.8 )$$

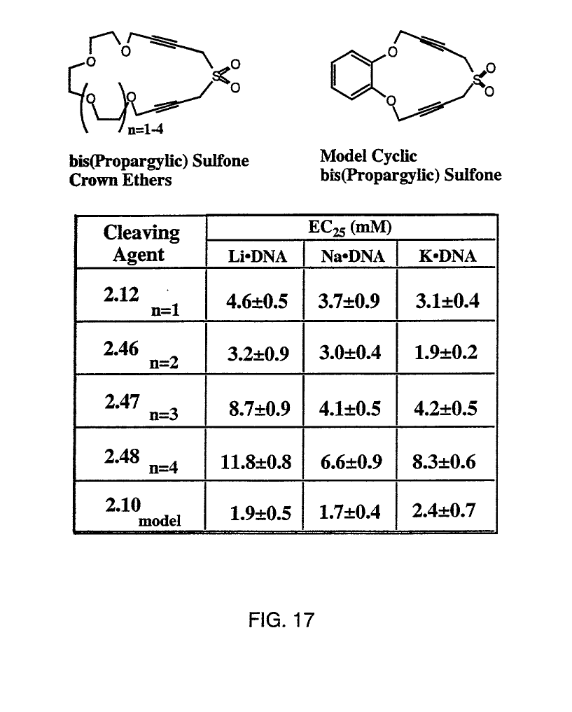

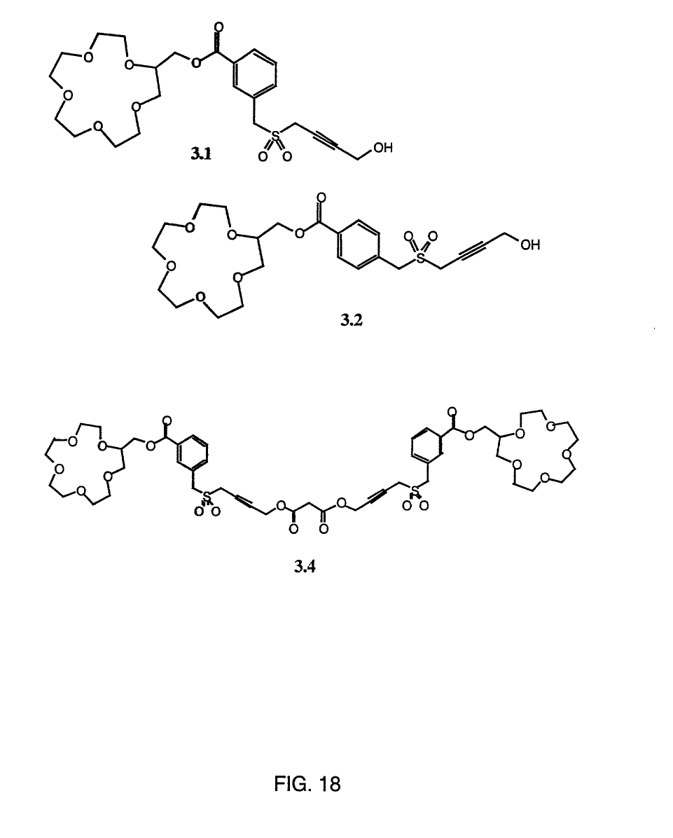

The results of the DNA cleavage assays for the bis(propargylic)sulfone crown ethers 2.12, 2.46, 2.47, 2.48, and the model bis(propargylic) sulfone 2.10 with the M.DNAs are presented in FIG. 17. The presented EC₂₅ values and their associated errors are the result of four or more separate determinations.

The smallest homologue of the bis(propargylic) sulfone crown series, 2.12, cleaves K.DNA a little more efficiently than it cleaves Li.DNA, while the efficiency of cleavage of Na.DNA lies roughly in between (see FIG. 17) The next higher homologue, 2.46, while equipotent at cleaving Li.DNA and Na.DNA, exhibits efficient cleavage of K.DNA (see FIG. 17). The next larger homologue, compound 2.47, cleaves Na.DNA and K.DNA similarly and with much greater efficiency than it cleaves Li.DNA (see FIG. 17). The last homologue of the bis(propargylic) sulfone crown ether series, 2.48, cleaves Na.DNA to a slightly greater extent than K.DNA, while Li.DNA is cleaved poorly (see FIG. 17).

The growth inhibitory activities of bis(propargylic) sulfone crown ethers 2.47 and 2.48 against B16 murine melanoma cells were determined calorimetrically with an MTT assay conducted by the Institute for Drug Development at the Cancer Research and Therapy Center in San Antonio, Tex. Compound 2.47 was found to have an IC₅₀ of 54.2 μM. Compound 2.48 was found to have an IC₅₀ of 44.7 μM. Compounds 2.47 and 2.48 also display growth inhibitory properties against a wide range of human cancer cell lines, including leukemia, non-small cell lung, prostate, melanoma, breast, ovarian, renal, colon, and CNS cancers ( See Table 1).

In another embodiment, crown ether macrocycles which contain functional appendages^{3.4} may be used as the metal binding moiety for a metal binding DNA interactive compound. These appendages, or pendent groups, may interact with a crown ether-complexed metal ion (as a Lewis acid in catalysis, for example). Alternatively, the pendent group may possess a function that is distinct from metal ion recognition; thereby creating a bifunctional agent. In one embodiment, the metal binding DNA interactive compound is a 15-crown-5-containing agent in which a DNA-cleaving propargylic sulfone linked pendent to the crown ether. The linking group in these examples is the phenyl ester. Examples of these pendent propargylic sulfone crown ethers, 3.1, 3.2, and 3.4 are depicted in FIG. 18.

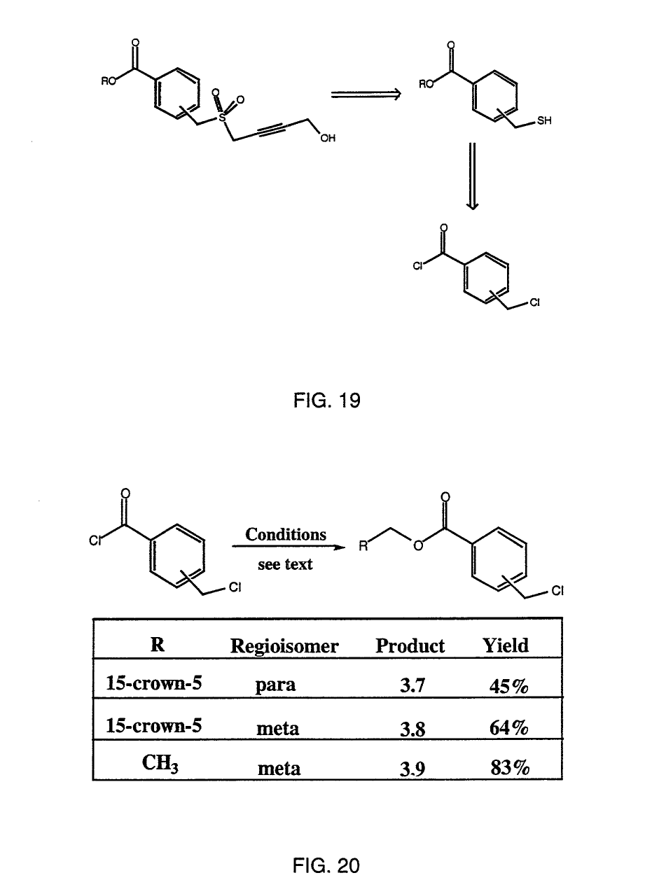

A strategy for the synthesis of propargylic sulfones 3.1, 3.2 and 3.3 is depicted retrosynthetically in FIG. 19. Pendent propargylic sulfone crown ethers may be prepared from chloromethylbenzoyl chlorides and hydroxy-crown ethers by proceeding through an esterified thiol.

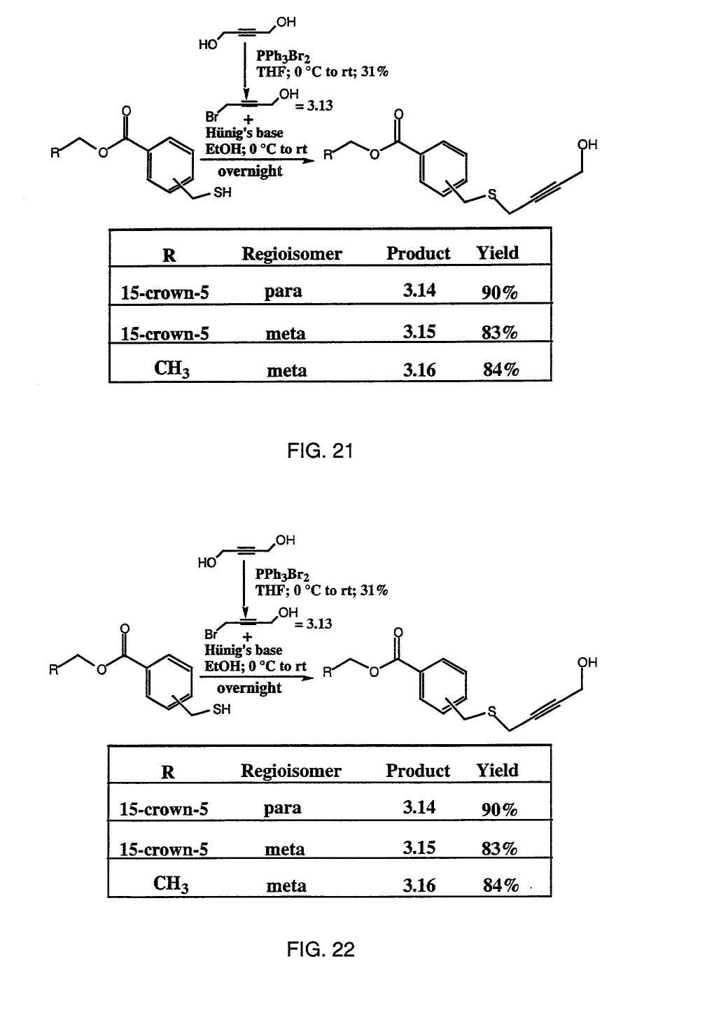

In one embodiment, esters 3.8 and 3.9 were prepared using standard conditions of DMAP catalysis in refluxing THF or DCM-containing THF, respectively. Ester 3.7 could be prepared using an analogous reaction in which the potassium alkoxide of 2-hydroxy-15-crown-5 was quenched with the acylium adduct of 4-chloromethylbenzoyl chloride and DMAP. The results of these esterifications are presented in FIG. 20. The esterified products may be converted into isothiuronium salts. These salts may be decomposed with n-BuNH₂ in cold ethanol to give the thiol derivatives 3.1 and 3.2. The results of these thiol formation reactions are depicted in FIG. 21. The thiols were alkylated with 4-bromo-2-butynol, 3.13. Thiol alkylation may be performed by cooling bromide 3.13 to (0° C.) and adding it to ethanolic solutions of the thiols in the presence of Hünig's base. The results of these thiol alkylation reactions are presented in FIG. 22.

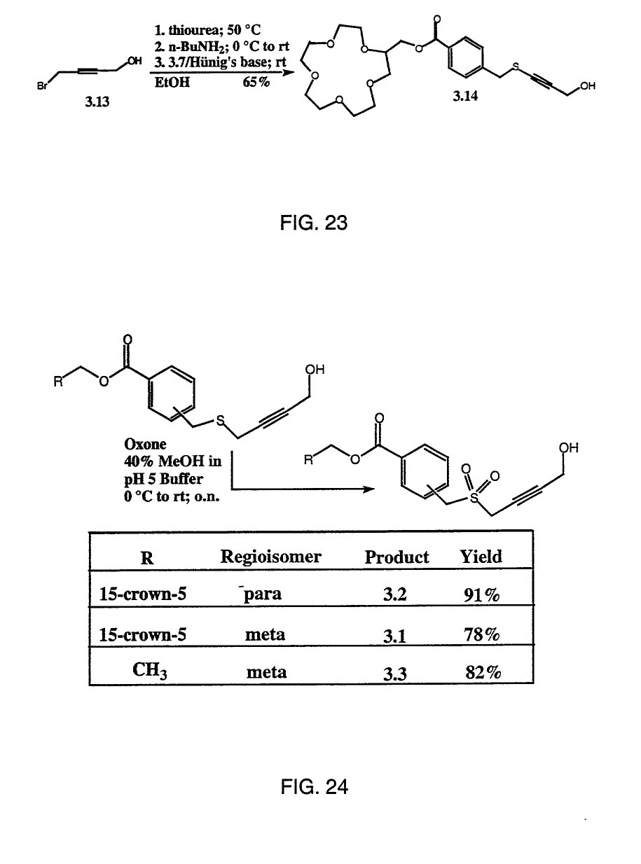

In another embodiment, intermediate sulfides (e.g. 3.14, see FIG. 22) may be obtained directly from the intermediate chlorides (e.g., 3.7 see FIG. 20). In a single step, a propargyl bromides may be treated with thiourea in hot ethanol, cooled and then decomposed to the thiol with n-BuNH_{2.}. The nascent thiol may be alkylated in situ with intermediate chloride (e.g., 3.7) to afford sulfides (e.g., 3.14). This reaction is depicted in FIG. 23.

The sulfides may be oxidized to the sulfones. Oxidation of the propargylic sulfide to the propargylic sulfones may be accomplished by treatment with an oxidizing agent. In one embodiment, oxidation of the propargylic sulfides to the corresponding propargylic sulfones was accomplished in high yield with the chemoselective oxidant Oxone® in buffered aqueous methanol. Some examples of this reaction are depicted in FIG. 24.

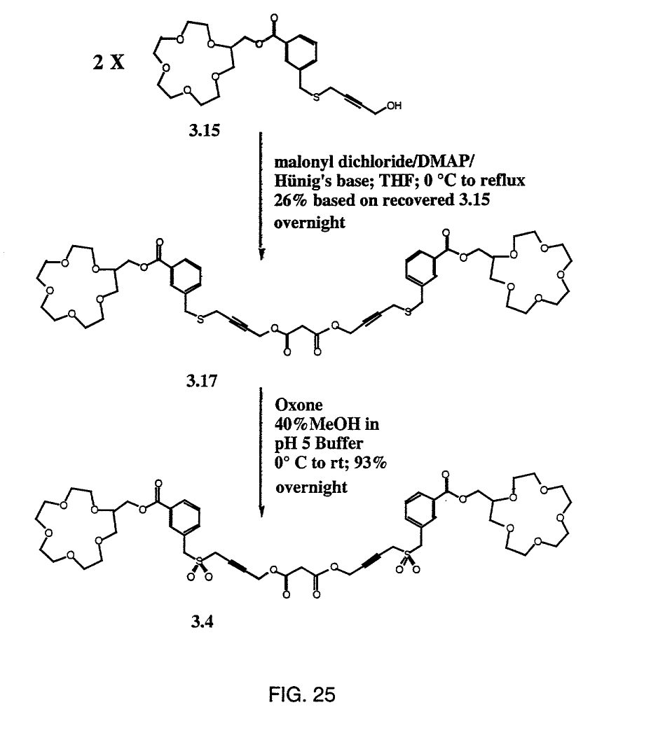

In another embodiment, the bis(propargylic sulfone) 2.4 may be synthesized using an analogous route. In one embodiment, two equivalents of sulfide 3.15 may be condensed with a mixture of malonyl dichloride and DMAP in THF to afford bis(propargylic sulfide) 3.17. Treatment of 3.17 with excess Oxone® afforded, after aqueous workup, pure bis(pendent propargylic sulfone crown ether) 3.4 in excellent yield (see FIG. 25).

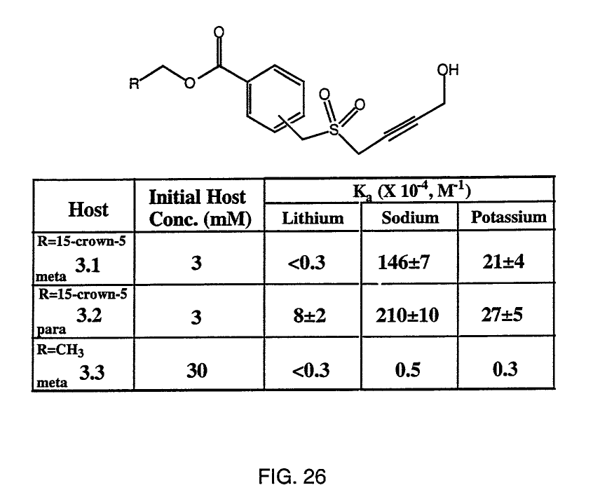

The complex association constants for propargylic sulfones 3.1, 3.2 and 3.3 with lithium, sodium or potassium ions are presented in FIG. 26. Each value represents the average of two separate determinations. The values listed in Table 3.5 which are preceded by a “less than” sign are the maximum theoretical values; the actual values may be lower. As FIG. 26 indicates, the 15-crown-5-containing propargylic sulfones 3.1 and 3.2 displayed very similar binding profiles. Host 3.2 exhibited greater affinity for all alkali metal ions versus host 3.1. Both crown ether-containing hosts exhibited nearly ten-fold greater affinity for sodium ions versus potassium ions, and slight affinity for lithium ions.

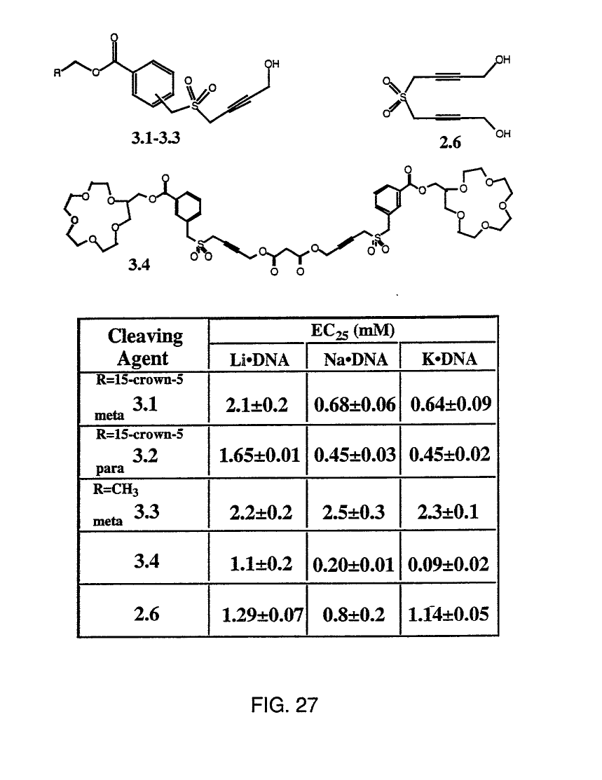

The DNA-cleavage capacity of propargylic sulfones was evaluated using the metallo-regulated DNA cleavage-assay previously described. Thus, propargylic sulfones 3.1, 3.2, 3.3, 3.4 and 2.6 were incubated with Li.DNA, Na.DNA and K.DNA, and EC₂₅ values were calculated as previously described. The results of the DNA cleavage assays for the pendent propargylic sulfone crown ethers 3.1 and 3.2, the model propargylic sulfone 3.3, the bis(pendent propargylic sulfone crown ether) 3.4, and the reference bis(propargylic) sulfone 2.6 with the M.DNAs (see section 2.6.1) are presented in FIG. 27. The presented EC₂₅ values and their associated errors (one standard deviation) are the average of three to five separate determinations.

In one example, pendent propargylic sulfone crown ethers 3.1 and 3.2 are more potent cleavers of Na.DNA and K.DNA than Li.DNA. The cleavage efficiency of propargylic sulfones 3.1, 3.2, and 3.3 nearly parallels the metal ion affinities of these compounds as determined by the picrate extraction assay. Thus, the 15-crown-5-containing agents display selectively enhanced cleavage of DNA samples which contain alkali metal ions (i.e., sodium and potassium) that are recognized by this crown ether.

The growth inhibitory activity of propargylic sulfone 3.2 against a wide range of human cancer cell lines was determined calorimetrically with sulforhodamine B assay conducted by the National Cancer Institute of the National Institutes of Health. Compound 3.2 was found to have modest growth inhibitory properties against certain human cancer cell lines (See Table 1).

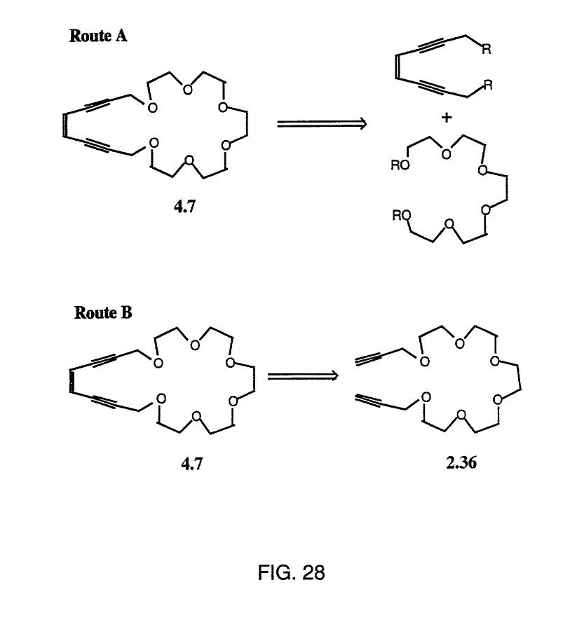

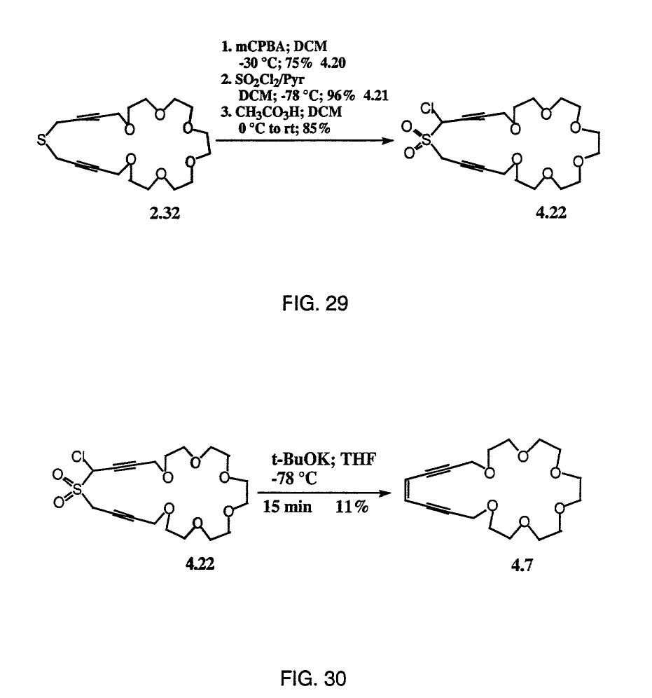

In another embodiment, a metal binding DNA interactive compound may be an enediyne crown ether. Enediyne crown ethers are composed of a enediyne moiety coupled to a crown ether. Two general routes for the synthesis of enediyne crown ethers are depicted in FIG. 28. Route A involves a double Williamson ether synthesis to form the macrocycle from enediyne-containing diol 4.11 and pentaethylene glycol with two leaving groups attached to the ends. The leaving groups may be halides or tosylates. Route B involves the use of a transition metal mediated coupling of two terminal acetylene groups with ehtylene dichloride to form the enediyne portion of the compound. A third strategy involves the decomposition of a bis(propargyl) sulfide crown ether to form the enediyne. This third strategy is depicted in FIGS. 29 and 30.

In one embodiment, a bis(propargylic) sulfide 2.32 was oxidized to sulfoxide 4.20 in high yield with a suitable oxidizing agent. In one embodiment, mCPBA may be used to oxidize the sulfide to sulfoxide. Sulfuryl chloride in pyridine-containing DCM may be used to halogenate the sulfoxide to α-chlorosulfoxide 4.21 Oxidation of 4.21 to α-chlorosulfone 4.22 may be accomplished with peracetic acid.

The α-chlorosulfone 4.22 may be decomposed using the Ramberg-Bäcklund route to an enediyne 4.7 (see FIG. 30). The Ramberg-Bäcklund route may be accomplished by the treatment of the α-chlorosulfone with a base. In one embodiment, potassium t-butoxide may be used to decompose the α-chlorosulfone to an alkene. In one embodiment, the formation of the enediyne moiety may be accomplished by the addition of a concentrated solution of t-BuOK in THF (2.5 eq.) in one portion to a precooled solution of the a-chlorosulfone (−78° C.; 35 mM), and quenching of the reaction after 15 min while it was still cold.

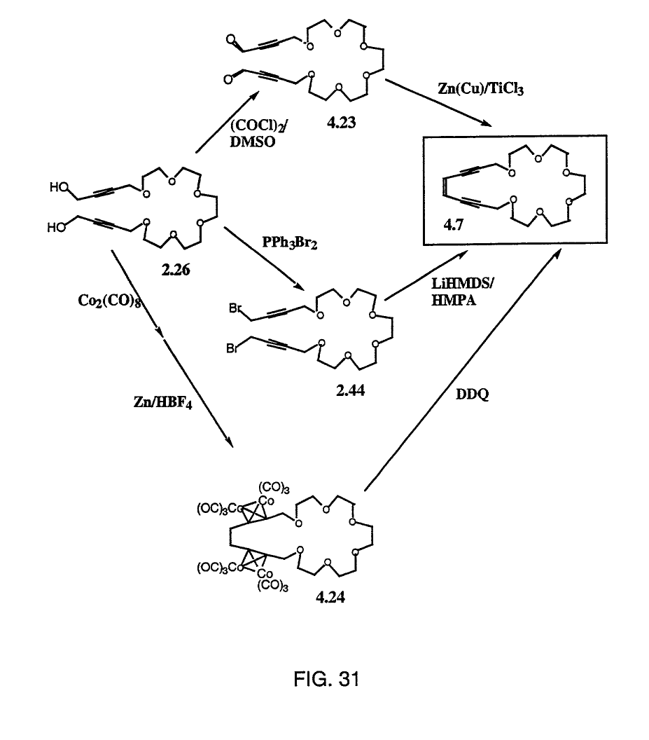

Alternate routes to enediyne crown ethers are depicted in FIG. 31. All three routes begin with a bis(propargylic) diol. The first route is based on the cyclization reaction of cobalt-protected bis(propargylic) alcohols via radical combination.^{4.26} In a on-pot procedure, diols may be treated with cobalt in the presence of Zinc to form a intermediate bis protected enediyne 4.24. This enediyne may be deprotected by the use of an oxidixing agent to form the crown ether enediyne.

In another route, depicted in FIG. 31, the diol may be oxidized to the dialdehyde, 4.23. This dialdehyde may be coupled in the presence of Zn and a Lewis acid (e.g., using a McMurray coupling protocol) to form the crown ether enediyne.

In a third route the diol 2.26 may be converted to the dibromide 2.44 as has been previously described. The dibromide may be converted to a enediyne by the treatment of the dibromide with a strong base. In one embodiment, a mixture of lithium hexamethyldisilazide may be used. Cosolvents such as HMPA or DMPU may be added to enhance the reaction.

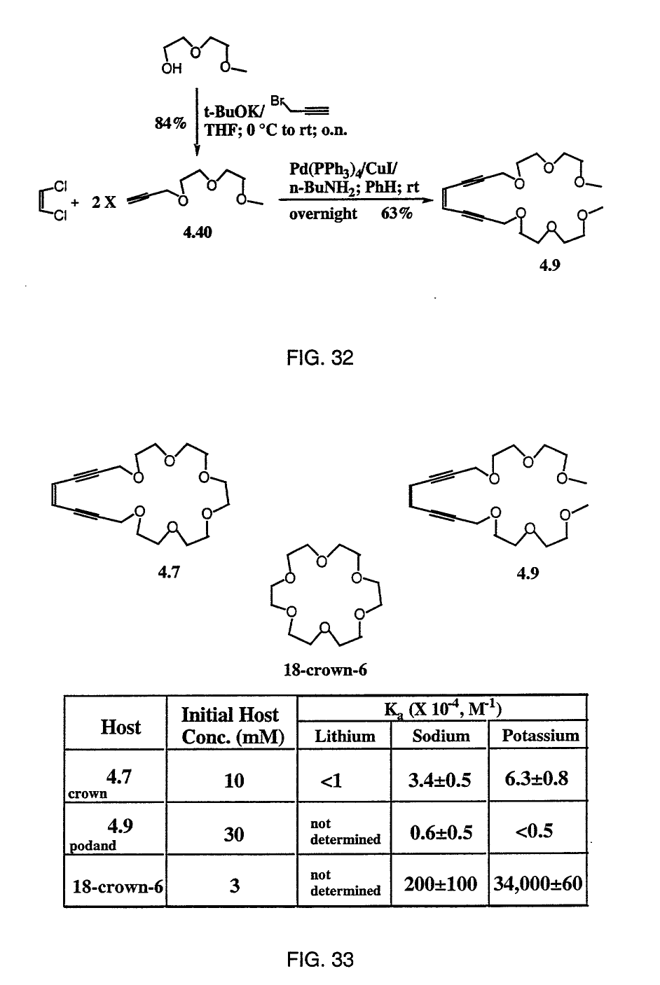

In another embodiment, enediyne-podands, such as 4.9 depicted in FIG. 32, may be used as a metal binding DNA interactive compound. Enediyne-podands are formed by the combination of polyethers with enediyenes to form an acyclic metal binding DNA interactive compound. These compounds may be synthesized by a number of different stratgies. In one embodiment, the enediyne podanads may be synthesized using a Williamson ether protocol in which an enediyne-containing diol is reacted with a polyether tosylate in the presence of a base.

In another embodiment, enediyne podands may be formed by a copper(I) and palladium(O)-catalyzed cross-coupling (Castro-Stephens reaction) between a propargyl ether and cis-ethylene dichloride as depicted in FIG. 32.

The metal binding properties of enediyne-crown 4.7 and enediyne-podand 4.9 were investigated using the picrate extraction method described above. The calculated complex association constants for enediyne-crown ether 4.7 with lithium, sodium and potassium, as well as those for enediyne-podand 4.9 and 18-crown-6 with sodium and potassium are presented in FIG. 33.

In one embodiment, the activity of the DNA interactive moiety may be influenced by the presence or absence of a metal ion bound to the metal ion binding moiety for crown ether enediynes. Cycloaromatization studies with enediyne-crown 4.7 and enediyne-podand 4.9 produced aromatic products arising from Bergman cyclization. Partial purification of the cyclization products was achieved using normal phase HPLC; spectroscopic identification was accomplished via ¹H-NMR and HRMS. Initially, the reaction progress of the Bergman cyclizations was monitored by HPLC (after the method reported by Nicolaou and co-workers⁵). A non-reactive component (caffeine) was included in the cyclization studies which served as an internal standard during HPLC analysis of aliquots. The aliquots were withdrawn at regular time intervals during the course of the reaction. The peak areas of remaining enediyne and internal standard were calculated from the chromatograms for each aliquot. The peak area ratio (PAR) of enediyne to internal standard was calculated and plotted versus time. In general, the plots so obtained appeared first order with respect to the starting enediyne. This allowed rate constants for the disappearance of starting material to be determined. The reaction may also be monitored using NMR.

The rate constants for the Bergman reaction of enediyne-crown ether 4.7 in the presence and absence of alkali metal salts (LiCl, NaCl, and KCl) was determined. The peak area ratio of enediyne to internal standard for seven time points (typically; including zero hours) was determined and plotted versus time. Graphically, the process revealed first order kinetics for disappearance of enediyne, given by equation 1 where PARₜ is the peak area ratio at time t (in hours), PAR₀ is the peak area ratio at time 0 hours (before heating), k is the first order rate constant for the Bergman reaction (in units of h⁻¹) and t is time in hours.

PARₜ=PAR₀e^{-kt} (1)

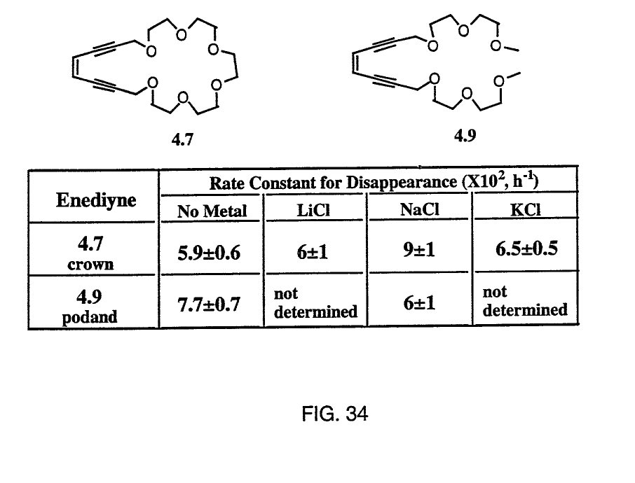

The graphical data was curve-fitted to an exponential equation to obtain the value of the slope, and thus the rate constant k. The rate constant for disappearance of enediyne-crown 4.7 and enediyne-podand 4.9 under four different conditions (no metal [D2O only], LiCl, NaCl and KCl) were determined. The composite data representing the average of at least two separate determinations for each condition studied is presented in FIG. 34.

Compound 4.7 exhibited an IC₅₀ of 68 μM for growth inhibition of B16 melanoma cells. As some weakly DNA-cleaving designed enediynes have exhibited the capacity to induce apoptosis in cancer cells^{4.43}, the possibility exists that compound 4.7 may exhibit inhibition of tumor cell growth by this mechanism as well.

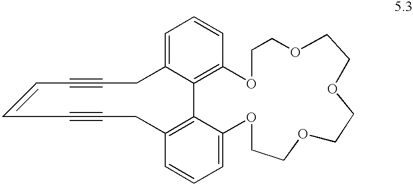

In another embodiment, biphenyl enediyne crown ethers (e.g., 5.3) may be used as a metal binding DNA interactive compound. These compounds are composed of a crown ether and an enediyne coupled together by a biphenyl, or biphenyl like aromatic system.

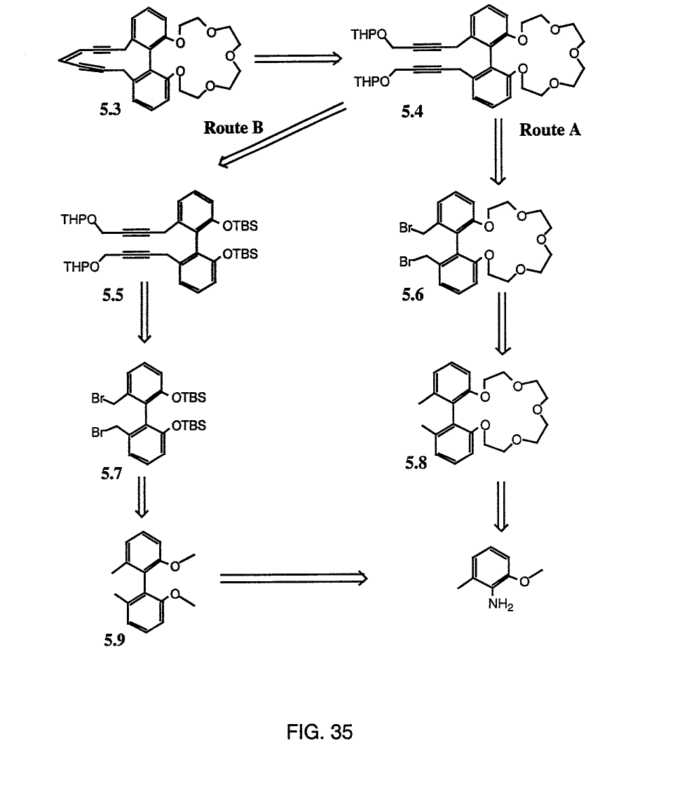

Biphenyl enediyne crown ethers may be formed by a number of different synthetic paths. In one embodiment, the synthesis of biphenyl enediyne crown ethers may be accomplished by the formation of the enediyene via a carbenoid coupling strategy, depicted in FIG. 35. Two routes may be used to get to this carbenoid coupling reaction. The routes differ in their synthetic sequences which lead to functionalization of the 2,2′ methyl groups prior to enediyne assembly. With route A, the crown ether may be constructed first, followed by benzylic functionalization (for example, via NBS-mediated bromination). With route B, oxidation and alkylation of the 2-position may be performed prior to crown ether assembly.

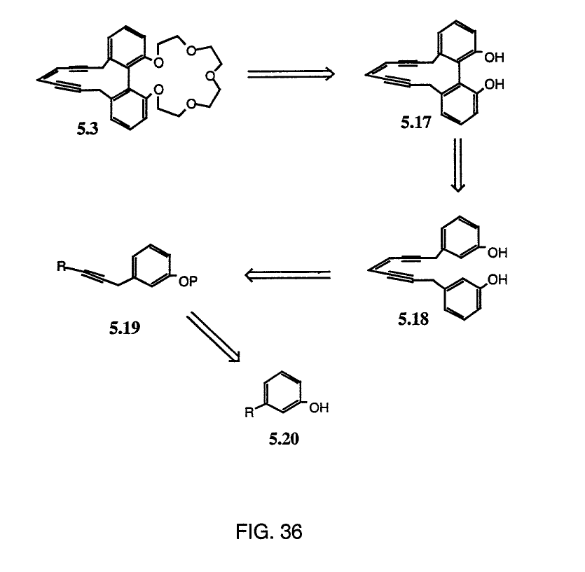

In another embodiment, biphenyl enediyne crown ethers may be synthesized by the methodology depicted in FIG. 36. The procedure begins with the alkylation of the aromatic ring to form a propargylated aromatic system 5.19. Next, the enediyne moiety may be constructed with a cis-olefin forming methodology (i.e., via Castro-Stephens coupling or Ramberg-Bäcklund reaction). Deprotection to the bis(phenol) and an oxidative coupling reaction to join the aromatic rings may be used to form the biphenyl substituent. Finally, the bis(phenol) may be alkylated with an appropriate polyether compound to afford the biphenyl enediyne crown ether.

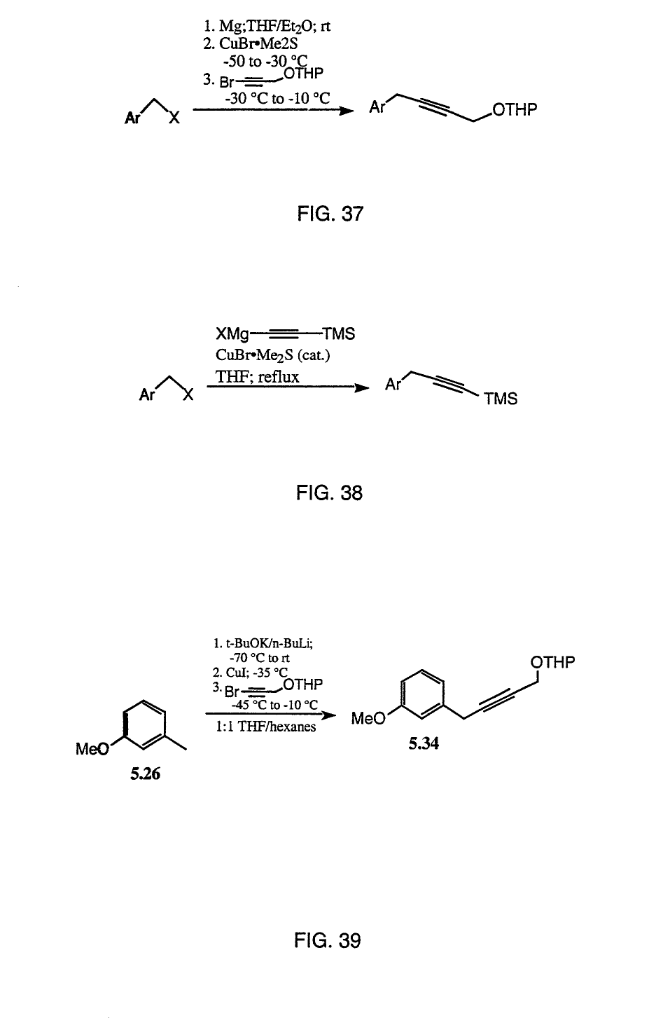

The alkylation of aromatic systems may be accomplished in a number of different ways. In one embodiment, a Grignard derived from a benzylic halide may be converted to the corresponding magnesium cuprate and alkylated with THP-protected 1-bromopropargyl alcohol as depicted in FIG. 37. Alternatively, a benzylic bromide substrate may be treated with an acetylenic Grignard reagent in the presence of catalytic copper (I) salts as depicted in FIG. 38. The 3-(aryl)-1-propyne thus obtained may be desilylated for subsequent use in the Castro-Stephens coupling reaction with cis-dihaloethylene to afford enediyne 5.18.

For either of the strategies depicted in FIGS. 37 and 38, a the hydroxy group of a phenolic benzylic halide may require protection prior to reaction with the propargylic group. A variety of protecting groups may be used, including THP, TBDMS, or methyl ethers. In one embodiment, a phenolic benzylic ether is synthesized by halogenation of a protected cresol system. Halogenation may be accomplished by treatment with a halogen source (e.g., NBS) in the presence of ultraviolet light. Such compounds may be used to synthesize intermediates of the type 5.19, depicted in FIG. 36, via the coupling reactions depicted in FIGS. 37 and 38

In another embodiment, the intermediate 5.19 may be synthesized by alkylation of a hydroxy protected cresol with a propargyl bromide, as depicted in FIG. 39. Alternatively, intermediate 5.19 may be synthesized by the conversion of a hydroxy protected 3-bromophenol to a Grignard reagent. The Grignard reagent is reacted methoxyallene in CuI-containing THF to give intermediate 5.19. The intermediate 5.19, where R is hydrogen, may be reacted under Castro-Stephens reaction conditions (See FIG. 32) with cis-dichloroethylene to form the enediyne intermediate 5.18. The enediyne intermediate 5.18 may be converted to product 5.3 via the strategy depicted in FIG. 36.

Turing back to FIG. 35, the synthesis of 5.3 according to Route B, depicted in FIG. 35, requires the initial synthesis of biphenyl 5.9. Compound 5.9 was first prepared by Sugii and Shindo in five steps from m-cresol.^{5.14} Alternatively, compound 5.9 could be formed by starting with commercially available 2-methoxy-6-methylaniline. Conversion of this aniline to 2-iodoanisole 5.10 occurred without incident in high yield via a diazotization/iodination sequence Iodide 5.10 may be converted to biphenyl 5.9 via an Ullmann reaction.

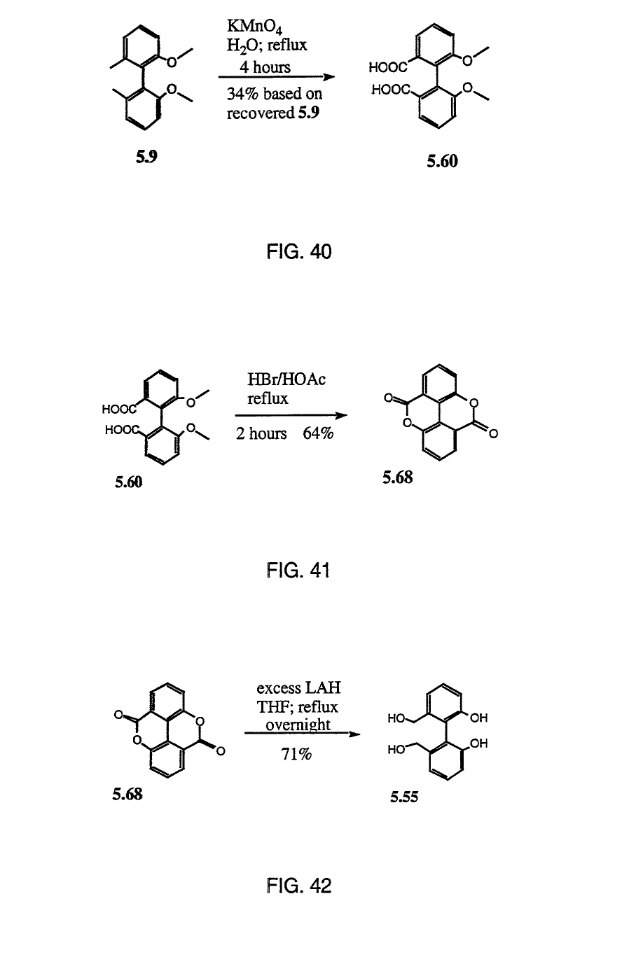

In one embodiment, compound 5.9 may be treated with an oxidizing agent (e.g., KMnO₄) to convert the alkyl groups to carboxylic acids as depicted in FIG. 40.

Intermediate 5.7 may be prepared by the deprotection of the hydroxyl groups of intermediate 5.60 and the reduction of the carboxylic acid groups. In one embodiment, the deprotection of the hydroxyl groups of 5.60 may be accomplished by heating the intermediate in the presence of an acid, depicted in FIG. 41. This reaction gives a dilactone intermediate 5.68 which may be reduced in the presence of a reducing agent (e.g., LiAlH₄) to give tetrol 5.55, see FIG. 42.

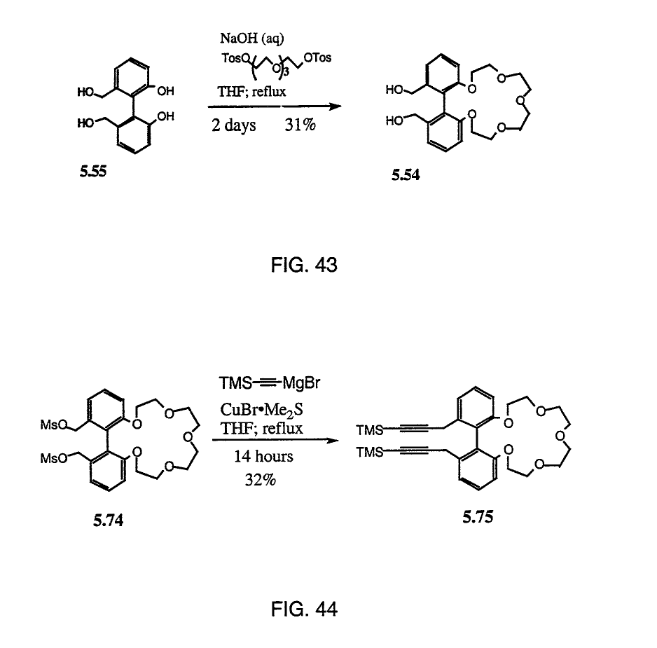

Tetrol 5.55 may serve as an intermediate for the formation of intermediate 5.4. In one embodiment, the crown ether portion of 5.4 may be formed by reaction of the tetrol with an ethylene glycol under substitution reaction conditions. For example, tetrol 5.55 may be reacted with tetraethylene glycol ditosylate to produce the crown ether biphenyl product 5.54 as depicted in FIG. 43.

The unreacted hydroxyl substitutions may be converted into an enediyne moiety by the following sequence. Initially, the unreacted hydroxyl groups are converted into a suitable leaving group (e.g., a mesylate) and reacted with a nucleophilic propargyl group to for the di propargylic intermediate 5.75 as depicted in FIG. 44.

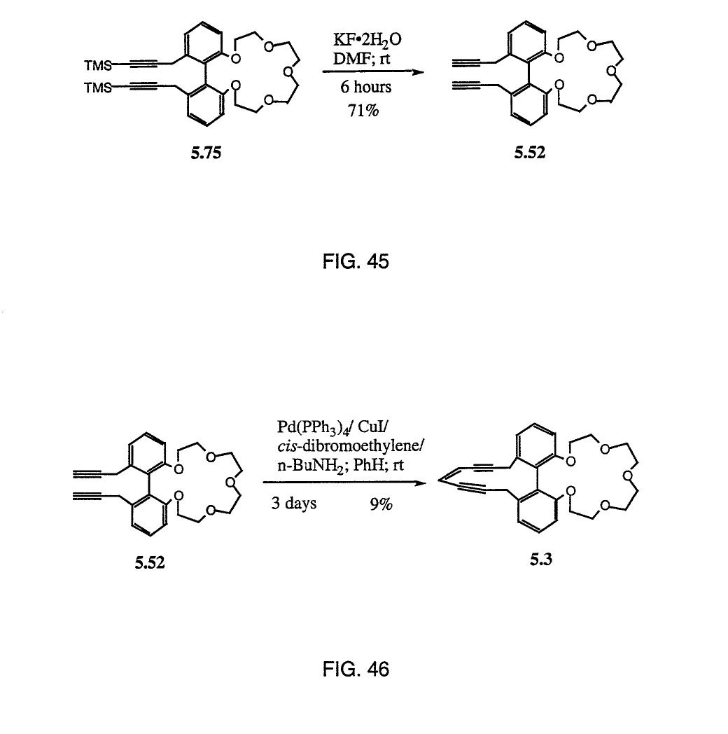

The synthesis of the biphenyl enediyne 5.3 may be completed using the Castro-Stephens methodology. Thus, the propargylic groups are deprotected by removal of the TMS groups, as depicted in FIG. 45. Coupling under the Castro-Stephens methodology with cis-dibromoethylene gives the desired product biphenyl enediyne 5.3, as depicted in FIG. 46.

Compound 5.3 was incubated with solutions of Na.DNA and Li.DNA at 37° C. for four days. The DNA fragments were electrophoretically separated, stained and digitally imaged as previously described. The extent of DNA cleavage due to compound 5.3 was extensive. Indeed, no Form I DNA remained when either M.DNA was employed. Additionally, while Form II and Form III cleavage fragments were prominent in the reaction with Li.DNA, these same DNA fragments were less visible in the reaction with Na.DNA.

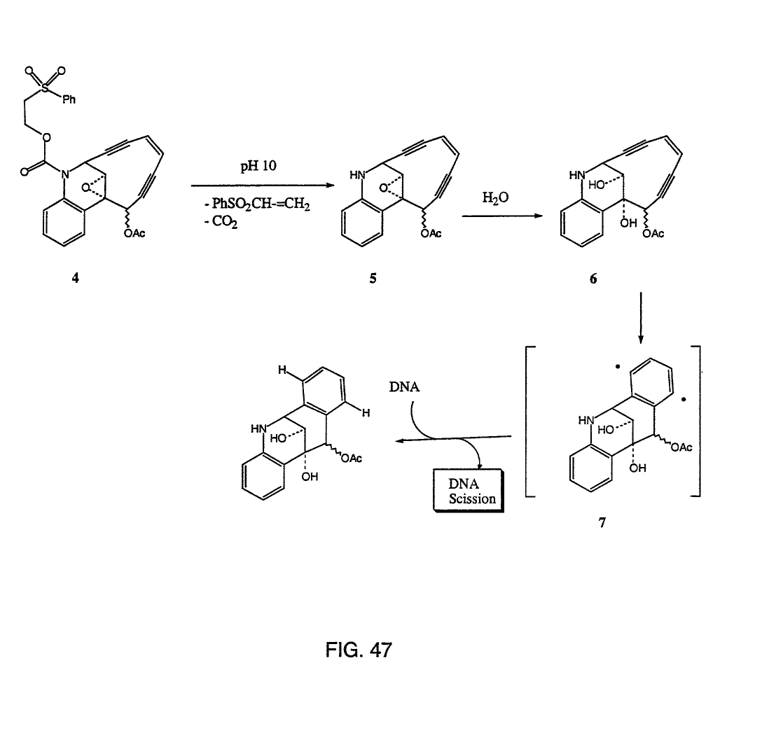

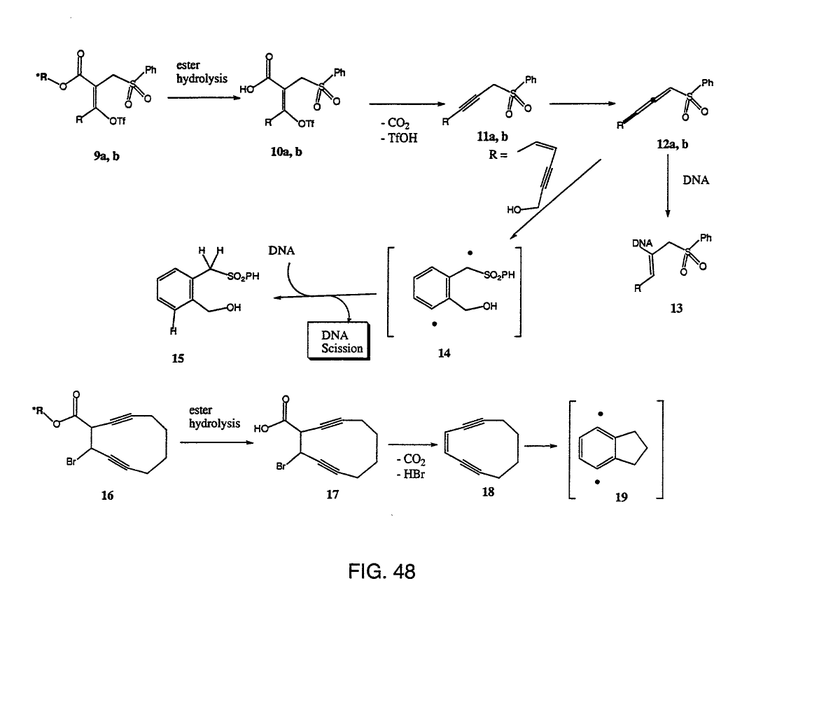

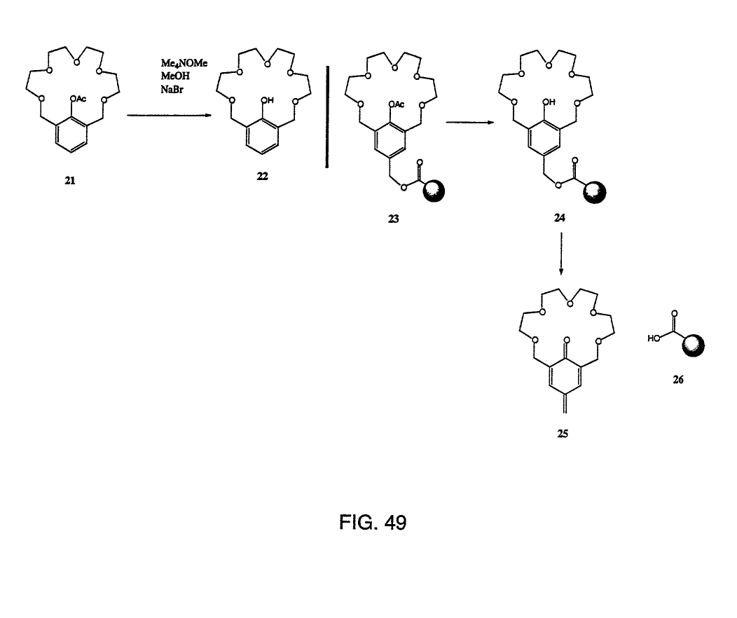

It has been reported that carbamate 4 (FIG. 47), an enediyne analog of dynemycin, undergoes base-promoted elimination from the sulfone to release the free amine 5. The resulting diol 6 then undergoes Bergman cyclization to afford the diradical intermediate 7, which cleaves DNA by hydrogen atom abstraction. In one embodiment, sulfone esters 9a, b, and β-bromoester 16 (R*═PhSO₂CH₂CH₂—) (FIG. 48) may be used as the DNA interactive moiety that is coupled with a alkali metal binding moiety. The base assisted hydrolysis of the ester linkage of these compounds should proceed as shown in FIG. 48. Once the ester group of 9a, b has been cleaved, the resulting acids 10a, b may undergo a facile decarboxylation-elimination to afford the alkynes 11a, b. Under the basic conditions necessary for ester hydrolysis, the alkynes 11a,b may isomerize to the allenes 12a, b. In the case of allene 12a (R═Ph), nucleophilic attack by DNA may afford the adduct 13, which may lead directly to DNA cleavage. Alternatively, the allene 12b may undergo a Myers-type cycloaromatization to produce the diradical 14, which may abstract hydrogen atoms from the DNA backbone resulting in DNA strand scission.

The ester 16 (R*═PhSO₂CH₂CH₂—), upon base-promoted ester hydrolysis, may afford the acid 17. Facile decarboxylative elimination gives rise to the nine-membered enediyne 18. Snyder has previously predicted that 18 undergoes Bergman cyclization under physiological conditions to produce the DNA-cleaving diradical species 19.

The DNA interactive moieties 9a,b and 16, depicted in FIG. 48, may be coupled to an alkali metal ion selective binding group. Some examples of such groups are depicted in FIG. 49. The crown ether 23 may display significant metal ion mediated ester hydrolysis. In the case of 23 the resulting phenol 24 may undergo elimination of the warhead carboxylate 26 to afford the quinonemethide intermediate 25.

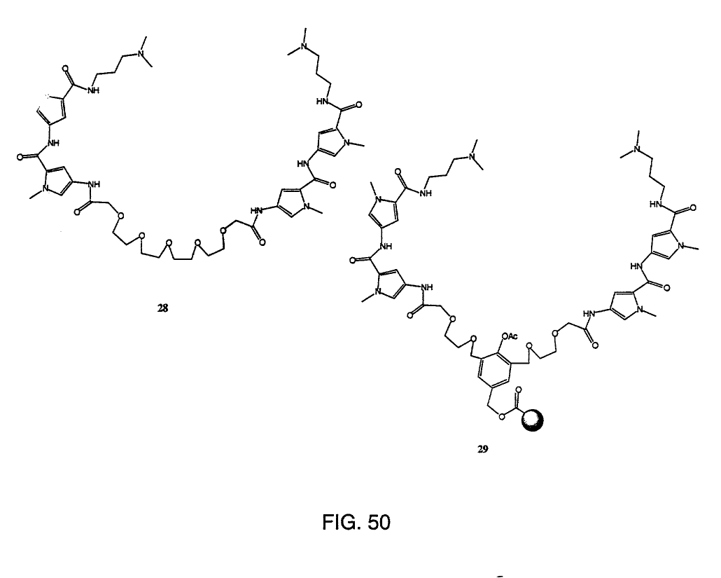

In another embodiment, a mechanism for the metal ion selective release of DNA reactive agents may be coupled with a group that exhibits sequence selective recognition of DNA. It is known that the polyethylene glycol-linked bis netropsin derivative 28 (FIG. 50) binds specifically in the presence of Ba²+ to DNA sequences consisting of two GIC base pairs flanked by 4 AIT base pairs. Compound 29 may form an appropriate host for Ba²+ when it binds to (A/T)₄(G/C)₂(A/T)₄ sites on DNA. The acetate group of 29 may be hydrolyzed relatively quickly when this molecule forms the ternary host-Ba²+DNA complex. The hydrolysis of 29 to the corresponding phenol may release the DNA reactive warhead and generate the potentially DNA-reactive quinonemethide.

The in vitro cytotoxicity of compounds 2.47, 2.48 and 3.2 were investigated using the DTP Human Tumor Cell Line Screen. The concept, rationale and history of development of the DTP Human Tumor Cell Line Screen were described by Boyd (1993;1997)^{8.4, 8.6} and Boyd and Paull (1995)^{8.5}. Further technical details of the NCI screening procedures were described (Monks, et. al.)^{8.2} as were the origins and processing of the cell lines (Alley, et. al., Shoemaker, et. al., Stinson, et. al.). Briefly, cell suspensions that were diluted according to the particular cell type and the expected target cell density (5000-40,000 cells per well based on cell growth characteristics) were added by pipet (100 μL) into 96-well microtiter plates. Inoculates were allowed a preincubation period of 24 h at 37° C. for stabilization. Dilutions at twice the intended test concentration were added at time zero in 100-μL aliquots to the microtiter plate wells. Usually, test compounds were evaluated at five 10-fold dilutions. In routine testing, the highest well concentration is 10E-4 M, but for the standard agents the highest well concentration used depended on the agent. Incubations lasted for 48 h in 5% CO₂ atmosphere and 100% humidity. The cells were assayed by using the sulforhodamine B assay (Rubinstein, et. al., Skehan, et. al.). A plate reader was used to read the optical densities, and a microcomputer processed the optical densities into the special concentration parameters.

| | IC₅₀ (μM) | IC₅₀ (μM) | IC₅₀ (μM) |
|---|---|---|---|
| | Compound | Compound | Compound |
| Panel/Cell Line | 2.47 | 2.48 | 3.2 |
| Leukemia | | | |
| HL-60 (TB) | 13.5 | 20.9 | >100 |
| K-562 | 11.2 | 23.9 | 37.0 |
| MOLT-4 | 3.7 | 19.9 | >100 |
| RPMI-8226 | — | 17.4 | 8.4 |
| SR | 8.2 | 18.9 | >100 |
| Non-Small Cell Lung | | | |
| Cancer | | | |
| A-549/ATCC | 20.2 | 61.7 | >100 |
| EKVX | 15.1 | 20.9 | >100 |
| HOP-62 | 21.7 | 21.0 | >100 |
| HOP-92 | 10.9 | 19.6 | 41.6 |
| NCI-H226 | 39.7 | 86.9 | >100 |
| NCI-H322M | 17.5 | 32.3 | 34.3 |
| NCI-H460 | 19.3 | 38.0 | >100 |
| NCI-H522 | 2.86 | 10.6 | 18.8 |
| Colon Cancer | | | |
| COLO 205 | 7.2 | 14.7 | >100 |
| HCC-2998 | 19.1 | 17.7 | >100 |
| HCT-116 | 8.1 | 15.3 | >100 |
| HCT-15 | 15.5 | 32.7 | >100 |
| HT29 | 13.8 | 18.7 | >100 |
| KM12 | 16.2 | 20.7 | >100 |
| SW-620 | 10.4 | 14.3 | 67.0 |
| CNS Cancer | | | |
| SF-268 | 24.2 | 30.5 | >100 |
| SF-295 | 32.0 | 81.2 | >100 |
| SF-539 | 11.1 | 26.2 | 46.8 |
| SNB-19 | 18.2 | 27.9 | >100 |
| SNB-75 | 19.9 | 32.6 | >100 |
| U251 | 16.9 | 18.3 | 69.0 |
| Melanoma | | | |
| LOX IMVI | 11.1 | 19.9 | 54.3 |
| MALME-3M | 2.7 | 14.6 | >100 |
| M14 | — | 16.9 | >100 |
| SK-MEL-2 | 16.1 | 17.3 | 98.7 |
| SK-MEL-28 | 18.9 | 17.9 | >100 |
| SK-MEL-5 | 16.8 | 18.1 | >100 |
| UACC-257 | 11.6 | 21.2 | >100 |
| UACC-62 | 0.28 | 17.1 | 82.4 |
| Ovarian Cancer | | | |
| IGROV1 | 16.3 | 44.6 | >100 |
| OVCAR-3 | 15.8 | 19.7 | >100 |
| OVCAR-4 | 19.8 | 27.3 | >100 |
| OVCAR-5 | 17.1 | 18.0 | >100 |
| OVCAR-8 | 14.2 | 33.3 | >100 |
| SK-OV-3 | 36.0 | 32.2 | >100 |
| Renal Cancer | | | |
| 786-0 | 11.0 | 18.4 | 35.3 |
| A498 | 16.9 | 54.7 | >100 |
| ACHN | 13.5 | 22.4 | >100 |
| CAKI-1 | 12.2 | 21.2 | >100 |
| RXF 393 | 15.1 | 27.0 | >100 |
| SN12C | 13.2 | 18.8 | 30.3 |
| TK-10 | 13.9 | 23.0 | >100 |
| Prostate Cancer | | | |
| PC-3 | 18.6 | 43.4 | >100 |
| DU-145 | 32.8 | >100 | >100 |
| Breast Cancer | | | |
| MCF7 | 10.1 | 22.0 | >100 |
| MCF7/ADR-RES | 25.7 | >100 | >100 |
| MDA-MB-231/ATCC | 20.0 | 23.5 | >100 |
| HS 578T | 40.7 | 52.8 | 80.0 |
| MDA-MB-435 | 13.3 | 20.4 | >100 |
| MDA-N | 14.2 | 18.9 | 37.4 |
| BT-549 | 14.1 | 20.0 | >100 |
| T-47D | 20.6 | 24.5 | |

### EXAMPLES

General Procedures. Unless otherwise noted, all materials were obtained from commercial suppliers and were used without further purification. Diethyl ether and THF were distilled from sodium benzophenone ketyl immediately prior to use. CH₂Cl₂, hexanes, and MeCN were distilled from CaH₂ immediately prior to use. Benzene and toluene were distilled from sodium metal prior to use. Absolute ethanol was dried and stored over 4 Å sieves. Acetone and mesyl chloride were distilled from (CaSO₄) prior to use. Ethyl chloroformate and 1,4-cyclohexadiene were distilled and stored under argon prior to use. Tri-, tetra-, penta-, and hexaethylene glycol were purified by Kugelrohr distillation. TMEDA, DMPU, pyridine, HMPA, HMDS, n-BuNH₂, Hünig's base, and TEA were distilled from CaH₂ prior to use. SOCl₂ and SO₂Cl₂ were distilled from linseed oil and stored in glass-stoppered vessels under argon. (CH₂O)ₙ was dried and stored in a vacuum dessicator over P₂O₅. CuI was purified by the method of Linstrumelle et al.¹ Concentrations of n-BuLi and isobutylmagnesium bromide solutions were determined by titration against diphenyl acetic acid. CuI, CuCN, CuBr.Me₂S, and Pd(PPh₃)₄ were handled and dispensed into reaction vessels under an argon atmosphere in a glovebag. All reactions were performed under an inert atmosphere of either argon or nitrogen and all reaction vessels and glassware for handling reagents were oven-dried and cooled in a dessicator prior to use. Unless otherwise noted, temperatures refer to external baths. Unless otherwise noted, organic solutions were dried with NA₂SO₄ and concentrated with a rotary evaporator (20-30 mm). Reaction progress was routinely monitored by TLC and R_{F} values were determined using Merck 60 F₂₅₄ aluminum-backed silica gel plates. Preparative TLC was conducted with Merck 60 F₂₅₄ glass-backed silica gel plates. Flash column chromatograpy was performed with the indicated solvents using 40 μ silica gel after the method of Still, Kahn and Mitra.² Melting points (open capillary) were determined with a Thomas-Hoover Unimelt apparatus and are uncorrected. Unless otherwise noted, IR spectra were determined as thin films on NaCl plates using a Nicolet 550 spectrometer. Unless otherwise noted, ¹H and ¹³C NMR spectra were determined on a Bruker spectrometer operating at 250 and 62.89 MHz, respectively using CDCl₃ as the solvent. Unless otherwise noted, mass spectra were obtained by chemical ionization using methane as the ionizing gas. UV absorbances were measured with a Perkin-Elmer Lambda Bio 10 UV/Vis spectrometer.

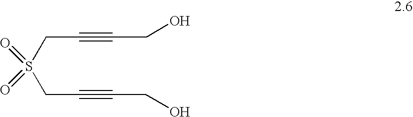

Compound 2.6:³ To a solution of sulfone 3.20 (0.015 g, 0.04 mmol) in 1.5 mL of EtOH was added PpTs (0.024 g, 0.096 mmol) and the resulting reaction mixture was heated to 50° C. with stirring for 7 h. Upon cooling to room temperature, the reaction mixture was diluted with EtOAc (20 mL) and 5:1 brine/water (12 mL). The layers were mixed, separated and the aqueous layer was extracted with EtOAc (3×10 mL). The combined organic layers were washed with brine (15 mL), dried, and the solvent was removed in vacuo. The residue was triturated with a small amount of CHCl₃ and the supernatant was removed to afford sulfone 2.6 (5 mg, 63%) as a colorless solid: R_{F}0.56 (EtOAc); ¹H NMR δ1.65 (s(br), 2H), 4.07 (t, J=2.0 Hz, 4H), 4.33 (t, J=2.0 Hz, 4H); ¹³C NMR (MeOH-d₄) δ44.39, 50.75, 73.04, 87.65; MS 203 (MH⁺), 185; HRMS m/e calc'd for C₈H₁₁O₄S: 203.0378, found 203.0365.

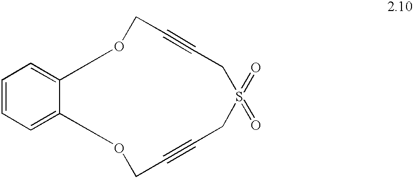

Compound 2.10:⁴ To a solution of sulfide 2.49 (60.0 mg, 0.246 mmol) in 2.3 mL of MeOH and 0.7 mL of CHCl₃ was slowly added dropwise a solution of Oxone® (977 mg, 0.787 mmol KHSO₅) in 2.3 mL of water and 1.1 mL of 2.5 M, pH 5 aqueous potassium citrate. The heterogeneous reaction mixture was allowed to stir at room temperature for 14 h and was then diluted with 85 mL of water in a separatory funnel. The reaction mixture was extracted with 3×50 mL of CHCl₃ and the combined organic layers were washed with 45 mL of water and 60 mL of brine. The organic layer was filtered and dried to afford sulfone 2.10 (60.7 mg, 89%) as a white solid: m.p. 176.5-177.5° C.; R_{F}0.09 (25% EtOAc in hexanes); ¹H NMR (d₈-dioxane) δ4.07 (s(br), 4H), 4.96 (s(br), 4H), 6.88-7.04 (m, 4H); ¹³C NMR (d₈-dioxane) δ46.41, 57.79, 76.93, 83.00, 119.32, 123.36, 148.70; IR 1339 cm⁻¹; MS 277 (MH⁺), 212, 161; HRMS m/e calc'd for C₁₄H₁₃O₄S: 277.0535, found 277.0538.

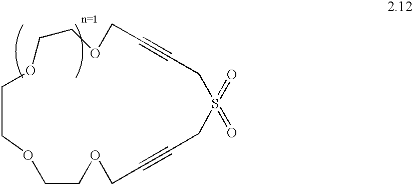

General procedure for sulfone formation. Compound 2.12⁵: To an ice-water bath-cooled solution of sulfide 2.18 (77.7 mg, 0.274 mmol) in 2.6 mL of MeOH was added dropwise a suspension of Oxone® (1.086 g, 0.877 mmol) in 2.6 mL of water and 1.24 mL of 2.5 M, pH 5 aqueous potassium citrate. Stirring was continued for an additional 18 h as the reaction mixture warmed to room temperature. The reaction mixture was diluted with 85 mL of water in a separatory funnel and extracted with 3×60 mL of CHCl₃. The chloroform extracts were washed with 55 mL of water and 65 mL of brine. Concentration of the organic layer afforded sulfone 2.12 (80.2 mg, 93%) as a white solid. An analytical sample recrystallized from 40% EtOAc in hexanes gave the following: m.p. 132-133° C.; R_{F}0.5 (5% MeOH in EtOAc); ¹H NMR δ3.58-3.74 (m, 12H), 4.12 (t, J=1.8 Hz, 4H), 4.27 (t, J=1.8 Hz, 4H); ¹³C NMR δ43.84, 58.55, 69.29, 70.32, 70.51, 73.35, 84.76; IR 1328 cm⁻¹; MS 317 (MH⁺); HRMS m/e calc'd for C₁₄H₂₁O₆S: 317.1059, found 317.1054.

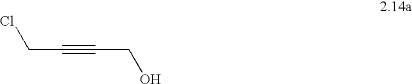

Compound 2.14a:⁶ A solution of 2-butyn-1,4-diol (17.2 g, 0.2 mol), pyridine (16.2 mL, 0.2 mol) and benzene (40 mL) in a 3-neck 500 mL round bottom flask equipped with a CaSO₄ drying tube, mechanical stirrer and pressure-equalized addition funnel was gently heated with stirring to facilitate dissolution of the diol. The reaction vessel was cooled 0° C. and SOCl₂ (14.5 mL, 0.2 mol) was added dropwise over 1 h with vigorous stirring while maintaining reduced temperature. Upon complete addition of SOCl₂, the cooling bath was allowed to melt and the reaction mixture was stirred for an additional 15 h. The reaction mixture was diluted with ice-water (50 mL) and Et₂O (50 mL) and the aqueous layer was extracted with 3×100 mL of Et₂O. The combined organic layers were washed with 100 mL of saturated aqueous NaHCO₃, 100 mL of ice-water, 100 mL of brine, dried (CaSO₄) and concentrated in vacuo. Kugelrohr distillation (78-79° C. ot, 1 torr) of the brownish residue afforded chloride 2.14a (10.0 g, 48%) as a colorless oil: ¹H NMR δ2.36 (s(br), 1H), 4.17 (t, J=2.2 Hz, 2H), 4.31 (t, J=2.2 Hz, 2H); ¹³C NMR δ30.28, 50.24, 79.96, 84.35; MS 105 (MH⁺), 70; HRMS m/e calc'd for C₄H₆ClO: 105.0107, found 105.0112.

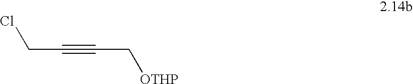

Compound 2.14b:⁶ To a solution of 2.14a (4.7 g, 45 mmol) in 300 mL of CH₂Cl₂ was added 3,4-dihydro-2H-pyran (7 mL, 77 mmol) and PpTs (1.13 g, 4.5 mmol) with stirring. The reaction mixture was stirred at room temperature for 4 h and then diluted with 250 mL of Et₂O and 150 mL of half-saturated brine. The organic layer was washed with 150 mL of half-saturated brine, 2×150 mL of brine, dried and concentrated in vacuo. Kugelrohr distillation (80-92° C. ot, 0.1 torr) of the residue gave acetal 2.14b (8.47 g, 99.9%) as a colorless oil: ¹H NMR δ1.46-1.91 (m, 6H), 3.48-3.59 (m, 1H), 3.77-3.89 (m, 1H), 4.18 (t, J=2.6 Hz, 2H), 4.26 (dt, J=16.4, 2.6 Hz, 1H)), 4.36 (dt, J=16.4, 2.6 Hz, 1H), 4.79 (t(br), J=3.4 Hz, 1H); ¹³C NMR δ18.57, 24.94, 29.77, 30.04, 53.69, 61.43, 80.23, 82.25, 96.44; MS 189 (MH⁺), 154, 104, 88; HRMS m/e calc'd for C₉H₁₄ClO₂ 189.0682, found 189.0682.

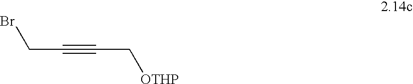

Compound 2.14c.⁷ From Compound 2.14b. To a stirring solution of chloride 2.14b (1.95 g, 10.3 mmol) in 15 mL of acetone was added NaBr (1.17 g, 11.4 mmol) and the resulting heterogeneous reaction mixture was heated to reflux for 24 h. The reaction mixture was allowed to cool and was then filtered under reduced pressure into a second reaction vessel containing NaBr (1.17 g, 11.4 mmol). The solids were washed with 5 mL of acetone into the second reaction vessel and the reaction mixture was again heated to reflux for 24 h. Three additional equilibrations with NaBr were performed in this manner. Upon cooling, the reaction mixture was filtered, the solids were washed with 5 mL of acetone, and the solvent was removed in vacuo. Kugelrohr distillation (100-110° C. ot, 4 torr) of the residue afforded 1.89 g of a pale yellow oil which was determined via ¹H-NMR to be a 4.9:1 mixture of 2.14c to 2.14b corresponding to a 67% yield of bromide 2.14c. In a similar procedure, chloride 2.14b was allowed to equilibrate nine times with NaBr; this gave a 13.7:1 mixture of 2.14c to 2.14b. However, the overall yield of bromide 2.14c was reduced to 50%.

From Compound 3.13. To a solution of bromide 3.13 (0.435 g, 2.9 mmol) and PpTs (0.089 g, 0.35 mmol) in 2.5 mL CH₂Cl₂ was added 3,4-dihydro-2H-pyran (450 μL 4.9 mmol) dropwise with stirring and the resulting reaction mixture was allowed to stir overnight at room temperature. The reaction mixture was diluted with diethyl ether (50 mL) and washed with 1:1 water/brine (20 mL), brine (25 mL) and dried (Na₂SO₄). The residue upon concentration and Kugelrohr distillation (100-110° C. ot, 4 torr) gave acetal 2.14c (0.594 g, 87%) as a light yellow oil: R_{F} 0.55 (20% EtOAc in hexanes); ¹H NMR δ1.42-1.87 (m, 6H), 3.46-3.56 (m, 1H), 3.74-3.86 (m, 1H), 3.93 (t, J=2.1 Hz, 2H), 4.24 (dt, J=16.3, 2.1 Hz, 1H), 4.33 (dt, J=16.3, 2.1 Hz, 1H), 4.76 (t(br), J=3.7 Hz, 1H); ¹³C NMR δ14.27, 18.88, 25.22, 30.09, 54.20, 61.89, 80.83, 82.85, 96.87; IR 1123, 1032, 625 cm⁻¹; MS 233 (MH⁺), 153, 131; HRMS m/e calc'd for C₉H₁₄BrO₂: 233.0177, found 233.0179.

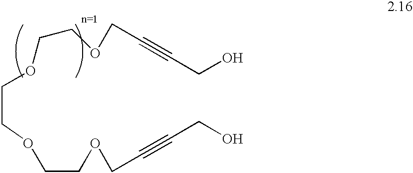

General procedure for hydroxymethylation. Compound 2.16:⁵ An argon-flushed 100 mL 3-neck round bottom flask equipped with a mechanical stirrer was charged with a solution of bis(propargyl ether) (593 mg, 2.62 mmol) in 22 mL of THF and TMEDA (4.0 mL, 26.5 mmol) and this was cooled with stirring to −78° C. in a dry ice/i-PrOH bath. n-BuLi (2.7 mL, 2.37 M, 6.39 mmol) was added dropwise with stirring and after an additional 5 min, a stirring suspension of paraformaldehyde (1.7 g, 56.6 mmol formaldehyde equivalents) in 7 mL of THF under argon was added via 18 gauge cannula quickly with good stirring. After an additional 5 min the cooling bath was removed and the reaction mixture was allowed to warm to room temperature and stir an additional 1 h. The light yellow, heterogeneous reaction mixture was transferred to a separatory funnel and diluted with 90 mL of EtOAc and 60 mL of saturated aqueous NaH₂PO₄. The layers were mixed, allowed to separate, and the aqueous layer was extracted with 3×45 mL EtOAc. The combined organic layers were washed with 60 mL of saturated NaHCO₃ and 65 mL of brine. The residue upon drying and concentration was purified by flash chromatography (2% MeOH in EtOAc) to afford diol 2.16 (293 mg, 39%) as a pale yellow solid: m.p. 33-34° C.; R_{F} 0.34 (2% MeOH in EtOAc); ¹H NMR δ2.95 (s(br), 2H), 3.60-3.73 (m, 12H), 4.20 (t, J=1.8 Hz, 4H), 4.27 (t, J=1.8 Hz, 4H); ¹³C NMR δ50.76, 58.62, 68.99, 70.37, 70.50, 81.27, 85.05; IR 3400 cm⁻¹; MS 287 (MH⁺), 269, 201, 157, 113; HRMS m/e calc'd for C₁₄H₂₃O₆: 287.1495, found 287.1485.

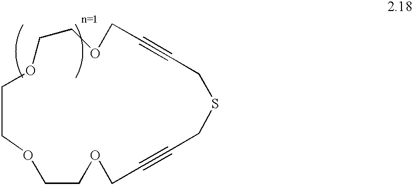

General procedure for macrocyclization. Compound 2.18:⁵ To a solution of dibromide 2.42 (150 mg, 0.364 mmol) in 24 mL of 5:1 CH₂Cl₂/EtOH was added in one portion Na₂.Al₂O₃ (0.311 g, 22% w/w Na2S, 0.877 mmol). The reaction mixture was blanketed with argon and allowed to stir at room temperature for 3 days. The heterogeneous reaction mixture was then filtered through Celite and the solids were washed with EtOAc. The combined eluant was concentrated, and the residue was purified by flash chromatography on silica gel (3% hexanes in EtOAc) to afford sulfide 2.18 (85 mg, 82%) as a colorless solid: m.p. 42-43° C.; R_{F} 0.52 (3% hexanes in EtOAc); ¹H NMR δ3.49 (t, J=1.9 Hz, 4H), 3.58-3.75 (m, 12H), 4.25 (t, J=1.9 Hz, 4H); ¹³C NMR δ19.13, 58.64, 68.78, 70.35, 70.51, 79.70, 81.23; IR 1142, 1100 cm⁻¹; MS 285 (MH⁺); HRMS m/e calc'd for C₁₄H₂₁O₄S: 285.1161, found 285.1164.

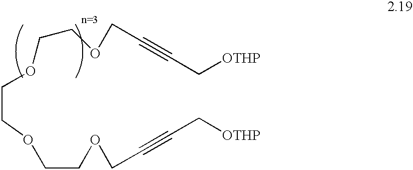

General procedure for alkylation with bromide 2.14c. Compound 2.19: To a solution of t-BuOK (95% w/w, 0.632 g, 5.36 mmol) in 45 mL of THF at room temperature was added a solution of pentaethylene glycol (0.638 g, 2.68 mmol) in 4 mL of THF via cannula with stirring over 1.5 min. After an additional five min, a solution of bromide 2.14c (95% w/w, 1.38 g, 5.6 mmol) in 4 mL of THF was added quickly via syringe with stirring over one minute. The resulting reaction mixture was allowed to stir at room temperature for 90 h. The reaction mixture was then diluted with 80 mL of brine and the aqueous layer was extracted with 3×35 mL of EtOAc. The combined organic layers were then washed with 3×50 mL of H₂O and 75 mL of brine, dried, and concentrated in vacuo. The obtained oil was purified by flash column chromatography on silica gel (5% MeOH in EtOAc) to afford compound 2.19 (0.453 g, 31%) as a light yellow oil: ¹H NMR δ1.43-1.88 (m, 12H), 3.45-3.55 (m, 2H), 3.59-3.69 (m, 20H), 3.75-3.86 (m, 2H), 4.22 (dt, J=15.2, 1.5 Hz, 2H), 4.22 (t, J=1.9 Hz, 4H), 4.32 (dt, J=15.2, 1.5 Hz, 2H), 4.77 (t(br), J=3 Hz, 2H); ¹³C NMR δ18.94, 25.24, 30.13, 54.17, 58.62, 61.89, 69.00, 70.32, 70.48 (2C), 70.51, 81.78, 82.34, 96.72; MS 543 (MH⁺), 457, 441, 417; HRMS m/e calc'd for C₂₈H₄₆O₁₀ 542.3091, found 542.3065.

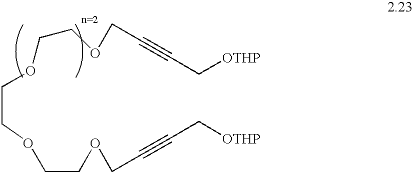

Compound 2.23: Following the general procedure (see compound 2.19), tetraethylene glycol (0.294 g, 1.51 mmol) gave a residue after workup that was purified by flash chromatography on silica gel (10% hexanes in EtOAc) to afford compound 2.23 (0.27 g, 36%) as a light yellow oil: ¹H NMR δ1.46-1.9 (m, 12H), 3.62-3.72 (m, 2H), 3.47-3.58 (m, 16H), 3.77-3.88 (m, 2H), 4.25 (dt, J=16.2, 2.1 Hz, 2H), 4.24 (t, J=2.1 Hz, 4H), 4.35 (dt, J=16.2, 2.1 Hz, 2H), 4.8 (t(br), J=3.9 Hz, 2H); ¹³C NMR δ19.02, 25.32, 30.2, 54.25, 58.7, 61.97, 69.09, 70.4, 70.56, 70.6, 81.85, 82.41, 96.81; MS 499 (MH⁺), 414, 397, 373; HRMS m/e calc'd for C₂₆H₄₁O₉: 497.2751, found 497.2737.

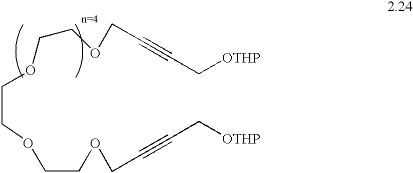

Compound 2.24: Following the general procedure (see compound 2.19), hexaethylene glycol (0.342 g, 1.21 mmol) gave a residue after workup that was purified by flash chromatography on silica gel (5% MeOH in EtOAc) to afford compound 2.24 (0.238 g, 34%) as a light yellow oil: ¹H NMR δ1.47-1.89 (m, 12H), 3.47-3.57 (m, 2H), 3.6-3.71 (m, 24H), 3.77-3.88 (m, 2H), 4.24 (dt, J=16.5, 1.9 Hz, 2H), 4.24 (t, J=1.9 Hz, 4H), 4.34 (dt, J=16.5, 1.9 Hz, 2H), 4.79 (t(br), J=2.9 Hz, 2H); ¹³C NMR δ19.02, 25.32, 30.21, 54.25, 58.70, 61.97, 69.08, 70.40, 70.56 (3C), 70.59, 81.85, 82.42, 96.81; MS 587 (MH⁺), 501, 485, 461; HRMS m/e calc'd for C₃₀H₅₁O₁₁: 587.3431, found 587.3421.

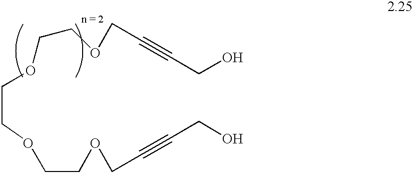

Compound 2.25. From compound 2.23. General procedure for deprotection. To a solution of 2.23 (0.504 g, 1.01 mmol) in 50 mL of EtOH was added PpTs (0.181 g, 0.71 mmol) with stirring. The reaction mixture was heated to 50° C. for 4 h and then allowed to cool to room temperature. The reaction solvent was removed in vacuo at room temperature and the residue was immediately purified by flash column chromatography on silica gel (5% MeOH in EtOAc) to afford diol 2.25 (0.244 g, 73%) as a pale yellow, heat-labile oil.

From compound 2.35. Following the general procedure (see compound 2.16), compound 2.35 (769 mg, 2.85 mmol) gave a residue after workup that was purified by flash chromatography on silica gel (5% MeOH in EtOAc) to afford diol 2.25 (450 mg, 48%) as a pale yellow oil: R_{F} 0.3 (5% MeOH in EtOAc); ¹H NMR δ2.86 (s(br), 2H), 3.60-3.70 (m, 16H), 4.20 (t, J=1.9 Hz, 4H), 4.26 (t, J=1.9 Hz, 4H); ¹³C NMR δ50.75, 58.60, 68.96, 70.37, 70.49 (2C), 81.28, 85.07; IR 3400 cm⁻¹; MS 331 (MH⁺), 313, 201, 157, 113; HRMS m/e calc'd for C₁₆H₂₇O₇: 331.1757, found 331.1756.

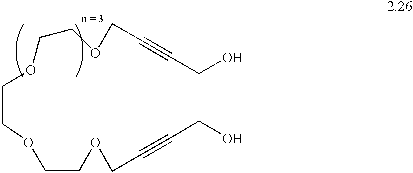

Compound 2.26.⁸ From compound 2.19. Following the general procedure (see compound 2.25), compound 2.19 (0.414 g, 0.764 mmol) gave a residue after workup that was immediately purified by flash chromatography on silica gel (10% MeOH in EtOAc) to afford diol 2.26 (0.223 g, 78%) as a pale yellow, heat-labile oil.

From compound 2.36. Following the general procedure (see compound 2.16), compound 2.36 (283 mg, 0.9 mmol) gave a residue after workup that was purified by flash chromatography (10% MeOH in EtOAc) to afford diol 2.26 (146 mg, 43%) as a pale yellow oil: R_{F} 0.32 (10% MeOH in EtOAc); ¹H NMR δ3.32 (s(br), 2H), 3.52-3.70 (m, 20H), 4.14 (t, J=1.7 Hz, 4H), 4.16-4.22 (m, 4H); ¹³C NMR δ50.19, 58.33, 68.67, 70.07, 70.21 (3C), 80.52, 85.12; IR 3443 cm⁻¹; MS 375 (MH⁺), 357, 245, 201, 157, 113; HRMS m/e calc'd for C₁₈H₃₁O₈: 375.2019, found 375.2016.

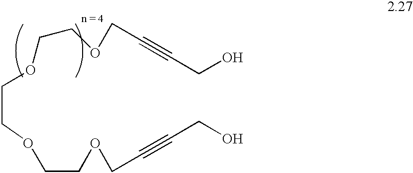

Compound 2.27. From compound 2.24. Following the general procedure (see compound 2.25), compound 2.24 (0.208 g, 0.355 mmol) gave a residue after workup that was immediately purified by flash chromatography on silica gel (15% MeOH in EtOAc) to afford diol 2.27 (0.114, 77%) as a pale yellow, heat-labile oil.

From compound 2.37. Following the general procedure (see compound 2.16), compound 2.37 (755 mg, 2.11 mmol) gave a residue after workup that was purified by flash chromatography on silica gel (10% MeOH in EtOAc) to afford compound 2.27 (357 mg, 40%) as a pale yellow oil: R_{F} 0.22 (10% MeOH in EtOAc); ¹H NMR δ2.70 (s(br), 2H), 3.58-3.70 (m, 24H), 4.20 (t, J=1.9 Hz, 4H), 4.26 (t, J=1.9 Hz, 4H); ¹³C NMR δ50.78, 58.61, 69.01, 70.38 (4C), 70.51, 81.32, 85.03; IR 3442 cm⁻¹; MS 419 (MH⁺), 401, 289, 245, 201; HRMS m/e calc'd for C₂₀H₃₅O₉: 419.2281, found 419.2284.

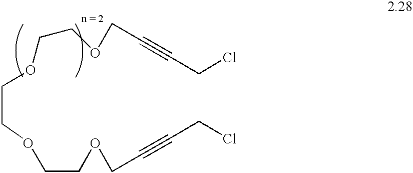

General procedure for chlorination. Compound 2.28: To a solution of diol 2.25 (0.245 g, 0.742 mmol) in 19 mL of THF was added HMPA (285 μL, 1.64 mmol) with stirring and this solution was cooled to 0° C. n-BuLi (1.86 M, 880 μL, 1.64 mmol) was added dropwise with stirring and the reaction mixture was allowed to warm to room temperature. This solution was then added dropwise over 5 minutes via cannula to a stirring solution of SOCl₂ (325 μL, 4.45 mmol) in 18 mL of THF. The reaction mixture was allowed to stir at room temperature for 15 minutes, pyridine (210 μL, 2.6 mmol) was added dropwise, and the resulting reaction mixture was heated to reflux for 16 h. The reaction mixture was allowed to cool to room temperature and was then diluted with 16 mL of H₂O, and the aqueous layer was extracted with 3×12 mL of EtOAc. The combined organic layers were washed with 2×20 mL of saturated aqueous NaHCO₃, 20 mL of H₂O, 20 mL of brine, dried and concentrated in vacuo to yield 0.293 g of a dark brown oil as a 4.6:1 mixture dichloride 2.28 to monochloride, corresponding to a 90% yield of dichloride 2.28. The mixture containing dichloride 2.28 was unstable to both silica gel and alumina column chromatography and was used without further purification. Analytical data for dichloride 2.28: ¹H NMR δ3.6-3.69 (m, 16H), 4.16 (t, J=1.8 Hz, 4H), 4.23 (t, J=1.8 Hz, 4H); ¹³C NMR δ30.24, 58.45, 69.12, 70.25, 70.43, 70.47, 80.99, 82.43; MS 367 (MH⁺), 331, 293; HRMS m/e calc'd for C₁₆H₂₅Cl₂O: 367.1079, found 367.107.

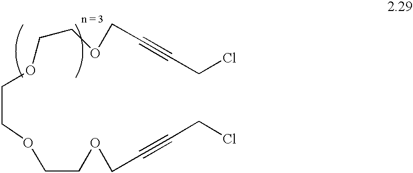

Compound 2.29⁸. Following the general procedure (see compound 2.28), diol 2.26 (0.328 g, 0.877 mmol) gave, after workup, spectroscopically pure dichloride 2.29 (0.319 g, 89%) as a dark brown oil. Dichloride 2.29 was unstable to both silica gel and alumina column chromatography and was used without further purification. Analytical data for dichloride 2.29: ¹H NMR δ3.6-3.7 (m, 20H), 4.16 (t, J=1.9 Hz, 4H), 4.23 (t, J=1.9 Hz, 4H); ¹³C NMR δ30.29, 58.52, 69.19, 70.32, 70.50 (2C), 70.54, 81.05, 82.49; IR 1135, 1094, 700; MS 411 (MH⁺), 375, 337; HRMS m/e calc'd for C₁₈H₂₉Cl₂O: 411.1341, found 411.1341.

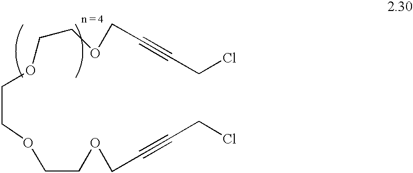

Compound 2.30: Following the general procedure (see compound 2.28), diol 2.27 (0.229 g, 0.548 mmol) gave, after workup, dichloride 2.30 (0.242 g, 98%) as a spectroscopically pure, dark brown oil. Dichloride 2.28 was unstable to both silica gel and alumina column chromatography and was used without further purification. Analytical data for dichloride 2.28: ¹H NMR δ3.6-3.69 (m, 24H), 4.16 (t, J=1.9 Hz, 4H), 4.23 (t, J=1.9 Hz, 4H); ¹³C NMR δ30.30, 58.55, 69.22, 70.35, 70.53 (4C), 81.07, 82.53; MS 455 (MH⁺), 419, 381; HRMS m/e calc'd for C₂₀H₃₃C₂O₇: 455.1603, found 455.1605.

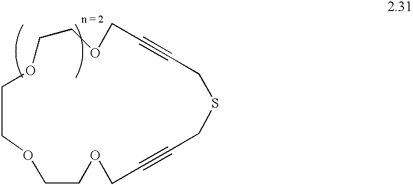

Compound 2.31: Following the general procedure (see compound 2.18), dibromide 2.43 (563 mg, 1.24 mmol) gave a residue after workup that was purified by flash chromatography on silica gel (EtOAc) to afford sulfide 2.31 (285 mg, 70%) as a pale yellow oil: R_{F} 0.36 (EtOAc); ¹H NMR δ3.47 (t, J=2.2 Hz, 4H), 3.62-3.73 (m, 16H), 4.23 (t, J=2.2 Hz, 4H); ¹³C NMR δ19.05, 58.69, 68.80, 70.41 (2C), 70.82, 79.60, 81.34; IR 1135, 1101 cm⁻¹; MS 329 (MH⁺); HRMS m/e calc'd for C₁₆H₂₅O₅S: 329.1423, found 329.1423.

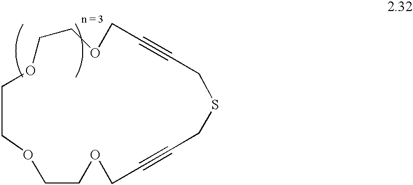

Compound 2.32:⁸ Following the general procedure (see compound 2.18), dibromide 2.44 (382 mg, 0.764 mmol) gave a residue after workup that was purified by flash chromatography on silica gel (5% MeOH in EtOAc) to afford sulfide 2.32 (213 mg, 75%) as a pale yellow oil: R_{F} 0.49 (5% MeOH in EtOAc); ¹H NMR δ3.47 (t, J=2.0 Hz, 4H), 3.63-3.72 (m, 20H), 4.23 (t, J=2.0 Hz, 4H); ¹³C NMR 19.17, 58.77, 68.94, 70.51, 70.71, 70.80, 70.87, 79.52, 81.42; IR 1141, 1099 cm⁻¹; MS 373 (MH⁺); HRMS m/e calc'd for C₁₈H₂₉O₆S: 373.1685, found 373.1682.

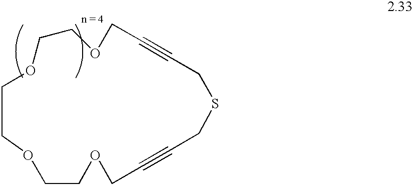

Compound 2.33: Following the general procedure (see compound 2.18), dibromide 2.45 (379 mg, 43% w/w mixture with PPh₃O, 0.3 mmol) gave a residue after workup that was purified by flash chromatography on silica gel (10% MeOH in EtOAc) to afford 30.9 mg of pure sulfide 2.33 as a colorless oil. Preparative TLC (1 mm silica gel plate, 10% MeOH in EtOAc) of 2.33-containing fractions that were contaminated with PPh₃O yielded an additional 20.7 mg of pure 2.33 (41% for combined material). Analytical data for compound 2.33: R_{F} 0.33 (10% MeOH in EtOAc); ¹H NMR δ3.46 (t, J=2.2 Hz, 4H), 3.62-3.70 (m, 24H), 4.23 (t, J=2.2 Hz, 4H); ¹³C NMR δ19.2, 58.69, 68.91, 70.43, 70.61 (2C), 70.67 (2C), 79.37, 81.43; IR 1115, 1097 cm⁻¹; MS 417 (MH⁺); HRMS m/e calc'd for C₂₀H₃₃O₇S: 417.1947, found 417.1944.

Na₂S.Al₂O₃ Reagent:⁸ To Na₂S.9H₂O (7.1 g, 0.03 mmol) that had been rinsed with a small amount of distilled, deionized water and placed in a flask under argon was added 18 mL of warm, distilled, deionized water that had been boiled to remove CO₂. The resultant solution was poured into a flask containing Al₂O₃ (neutral, Brockmann Activity I, 80-200 mesh, 8.7 g, 0.085 mmol), and the water was removed in vacuo via rotary evaporator with gentle heating in a warm water bath. The material was then activated by heating in vacuo (95° C., 0.1 torr) for 1.5 h until the material (21.2% w/w Na₂S) was a free-flowing pink powder. The reagent was stored under argon and used shortly after it was prepared.

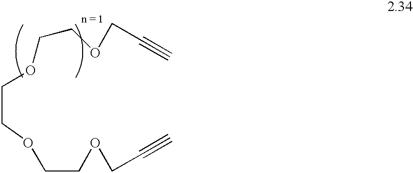

General procedure for bis(alkylation) using propargyl bromide. Compound 2.34.⁹ To a cooled (0° C.) suspension of t-BuOK (3.77 g, 95% w/w, 32 mmol) in 28 mL of TBF under argon in a 100 mL pear flask was added with stirring a solution of triethylene glycol (2.13 g, 14.2 mmol) in 3 mL of THF rather quickly via cannula. The resulting glycol dialkoxide was allowed to warm to room temperature and was then added via cannula with vigorous stirring over 26 min to an ice-water bath-cooled solution of propargyl bromide (6.3 mL, 80% w/w, 56.5 mmol) in 114 mL of THF under argon within a 250 mL 3-neck round bottom flask equipped with a mechanical stirrer. The reaction mixture was allowed to stir for an additional 18 h as the ice-water bath melted and the reaction was allowed to warm to room temperature. The thick cream-colored heterogeneous reaction mixture was transferred to a separatory funnel, diluted with 75 mL of 3:1 brine/water and the layers were mixed and allowed to separate. The aqueous layer was extracted with 3×50 mL of EtOAc and the combined organic layers were washed with 40 mL of 1:1 brine/water and 65 mL brine. The residue upon drying and concentration was purified by flash chromatography on silica gel (50% hexanes in EtOAc) to afford bis(propargyl ether) 2.34 (2.37 g, 74%) as a light golden oil: R_{F} 0.46 (50% hexanes in EtOAc); 1H NMR δ2.40 (t, J=2.5 Hz, 2H), 3.63-3.72 (m, 12H), 4.18 (d, J=2.5 Hz, 4H); ¹³C NMR δ58.05, 68.77, 70.08, 70.26, 74.35, 79.39; IR 2879, 2117, 1099 cm⁻¹; MS 227 (MH⁺), 171, 127; HRMS m/e calc'd for C₁₂H,₁₉O₄: 227.1283, found 227.1288.

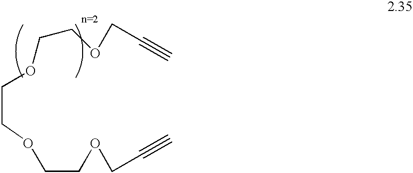

Compound 2.35:¹⁰ Following the general procedure (see compound 2.34), tetraethylene glycol (2.51 g, 12.9 mmol) gave a residue after workup that was purified by flash chromatography on silica gel (35% hexanes in EtOAc) to afford bis(porpargyl ether) 2.35 (2.67 g, 77%) as a light yellow oil: R_{F} 0.45 (35% hexanes in EtOAc); ¹H NMR δ2.37 (t, J=2.4 Hz, 2H), 3.53-3.63 (m, 16H), 4.10 (d, J=2.4 Hz, 4H); ¹³C NMR δ58.11, 68.83, 70.13, 70.31, 70.33 74.38, 79.43; IR 2872, 2117, 1101 cm⁻¹; MS 271 (MH⁺), 215, 171, 127; HRMS m/e calc'd for C₁₄H₂₃O₅: 271.1545, found 271.1542.

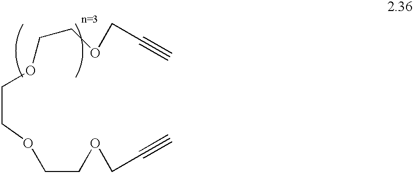

Compound 2.36:⁸ Following the general procedure (see compound 2.34), pentaethylene glycol (2.77 g, 11.6 mmol) gave a residue after workup that was purified by flash chromatography (EtOAc) to afford bis(propargyl ether) 2.36 (3.0 g, 82%) as a pale yellow solid: m.p. 36-37° C.; R_{F} 0.48 (EtOAc); ¹H NMR δ2.39 (t, J=2.3 Hz, 2H), 3.54-3.67 (m, 20H), 4.14 (d, J=2.3 Hz, 4H); ¹³C NMR δ58.23, 68.94, 70.24, 70.43, 70.45, 74.43, 79.52; IR, 2870, 2116, 1117 cm⁻¹; MS 315 (MH+), 277, 259, 215, 171, 127; HRMS m/e calc'd for C₁₆H₂₇O₆: 315.1808, found 315.1806.

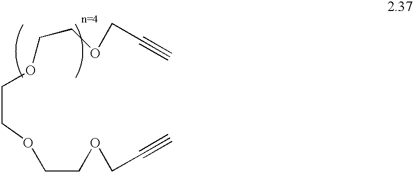

Compound 2.37: Following the general procedure (see compound 2.34), hexaethylene glycol (2.89 g, 10.2 mmol) gave a residue after workup that was purified by flash chromatography (5% MeOH on EtOAc) to afford bis(propargyl ether) 2.37 (3.11 g, 85%) as a yellow oil: R_{F} 0.53 (5% MeOH in EtOAc); ¹H NMR δ2.38 (t, J=2.4 Hz, 2H), 3.55-3.66 (m, 24H), 4.14 (d, J=2.4 Hz, 4H); ¹³C NMR δ58.22, 68.95, 70.24, 70.42 (4C), 74.42, 79.52; IR 2872, 2116, 1107 cm⁻¹; MS 359 (MH⁺), 259, 215, 171, 127; HRMS m/e calc'd for C₁₈H₃₁O₇: 359.2070, found 359.2060.

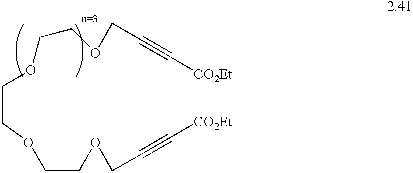

Compound 2.41: To a chilled (−78° C.) solution of bis(propargyl ether) 2.36 (0.116 g, 0.37 mmol) and DMPU (95 μL, 0.78 mmol) in 2 mL of THF was added n-BuLi (330 μL, 2.31 M, 0.76 mmol) with stirring. An additional 5 mL of THF was added and the reaction mixture was allowed to stir for 15 additional min. A solution of ethyl chloroformate (140 μL, 1.47 mmol) in 5 mL of THF was added via cannula over 15 sec and the resultant reaction mixture was allowed to stir an additional 30 min at −78° C. and then for 3 h at room temperature. The reaction mixture was quenched with 20 mL of saturated aqueous NH₄Cl, diluted with 20 mL of EtOAc and transferred to a separatory funnel. The layers were mixed, separated, and the aqueous layer was extracted with 3×12 mL of EtOAc. The combined organic layers were washed with 20 mL of brine. The residue upon drying and concentration was purified by flash chromatography on silica gel (EtOAc) to afford a pale yellow oil (0.108 g) which contained 87% w/w diester 2.41 (as determined by ¹H-NMR; corresponding to 55% yield of diester 2.41) contaminated with starting compound 2.36. Analytical data for diester 2.41: R_{F} 0.53 (EtOAc); ¹H-NMR δ1.25 (t, J=7.2 Hz, 6H), 3.54-3.70 (m, 20H), 4.18 (q, J=7.2 Hz, 4H), 4.28 (s, 4H); ¹³C-NMR δ13.88, 58.10, 61.99, 69.54, 70.24, 70.45, 70.48, 70.54, 78.04, 83.08, 152.98.

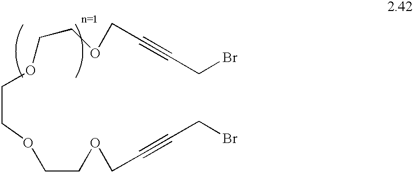

General procedure for bromination. Compound 2.42: To an ice-water bath-cooled solution of PPh₃ (457 mg, 1.74 mmol) in mL of CH₂Cl₂ under argon was added Br₂ (87 μL, 1.7 mmol) dropwise via gastight syringe with good stirring to afford an off-white heterogeneous suspension of PPh₃Br₂. After an additional 5 min, a solution of diol 2.16 (202 mg, 0.707 mmol) in 1.9 mL of CH₂Cl₂ was added dropwise via cannula over 3 min, and the resulting reaction mixture was stirred for an additional 5 h as the reaction mixture was allowed to warm to room temperature. The light golden-yellow, homogeneous reaction mixture was transferred to a separatory funnel and diluted with 40 mL of EtOAc and 15 mL of saturated aqueous NaHCO₃. The layers were mixed, allowed to separate, and the aqueous layer was extracted with 3×10 mL of EtOAc. The combined organic layers were washed with 20 mL of brine, dried, and concentrated to a residue that was purified by flash chromatography on silica gel (3% hexanes in EtOAc) to afford dibromide 2.42 (239 mg, 82%) as a pale yellow oil: R_{F} 0.81 (3% hexanes in EtOAc); ¹H NMR δ3.59-3.65 (m, 12H), 3.90 (t, J=2.0 Hz, 4H), 4.20 (t, J=2.0 Hz, 4H); ¹³C NMR δ14.18, 58.56, 69.14, 70.29, 70.48, 81.30, 82.79; IR 617 cm⁻¹; MS 411 (MH⁺), 263, 219, 175; HRMS m/e calc'd for C₁₄H₂₁Br₂O₄: 410.9807, found 410.9802.

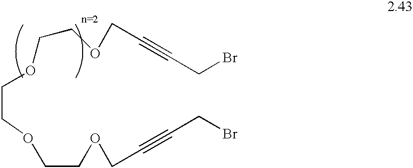

Compound 2.43: Following the general procedure (see compound 2.42), diol 2.25 (220 mg, 0.668 mmol) gave a residue after workup that was purified by flash chromatography on silica gel (EtOAc) to afford dibromide 2.43 (275 mg, 90%) as a pale yellow oil: R_{F} 0.73 (EtOAc); ¹H NMR δ3.55-3.65 (m, 16H), 3.90 (t, J=2.0 Hz, 4H), 4.20 (t, J=2.0 Hz, 4H), ¹³C NMR δ14.15, 58.54, 69.13, 70.27, 70.45, 70.48, 81.28, 82.78; IR 614 cm⁻¹; MS 456 (MH⁺), 377, 309, 265, 221, 177; HRMS m/e calc'd for C₁₆H₂₅Br₂O₅: 455.0069, found 455.007.

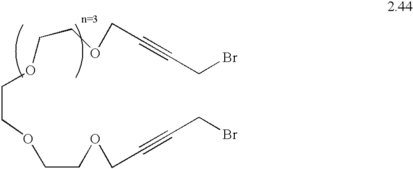

Compound 2.44:⁸ Following the general procedure (see compound 2.42), diol 2.26 (108 mg, 0.288 mmol) gave a residue after workup that was purified by flash chromatography on silica gel (EtOAc) to afford dibromide 2.44 (117 mg, 81%) as a colorless solid: m.p. 31.5-32.5° C.; R_{F} 0.48 (EtOAc); ¹H NMR δ3.59-3.70 (m, 20H), 3.93 (t, J=2.0 Hz, 4H), 4.24 (t, J=2.0 Hz, 4H); ¹³C NMR δ14.12, 58.50, 69.10, 70.24, 70.43 (2C), 70.45, 81.26, 82.76; IR 620 cm⁻¹; MS 499 (MH⁺), 419, 309, 263, 219; HRMS m/e calc'd for C₁₈H₂₉Br₂O₆; 499.0331, found 499.0323.

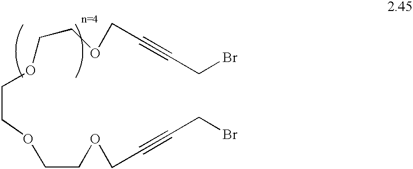

Compound 2.45: Following the general procedure (see compound 2.42), diol 2.27 (427 mg, 1.02 mmol) gave a light tan solid after workup that contained dibromide 2.45 (1.17 g, 43% w/w 2.45, 90%) and PPh₃O as an inseparable mixture. Analytical data for dibromide 2.45: R_{F} 0.56 (5% MeOH in EtOAc); ¹H NMR δ3.55-3.70 (m, 24H), 3.93 (t, J=2.2 Hz, 4H), 4.24 (t, J=2.2 Hz, 4H); ¹³C NMR δ14.20, 58.66, 69.26, 70.39, 70.60 (4C), 81.39, 82.91; MS 544 (MH⁺), 307; HRMS m/e calc'd for C₂₀H₃₃Br₂O₇: 543.0593, found 543.0583.

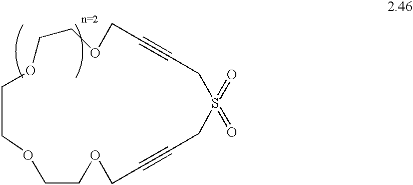

Compound 2.46: Following the general procedure (see compound 2.12), sulfide 2.31 (29.3 mg, 89.3 μmol) gave, after workup, sulfone 2.46 (29.9 mg, 93%) as a colorless solid. An analytical sample recrystallized from 40% EtOAc in hexanes gave the following: m.p. 72.5-73.5° C.; R_{F} 0.54 (10% MeOH in EtOAc); ¹H NMR δ3.57-3.72 (m, 16H), 4.10 (t, J=1.8 Hz, 4H), 4.26 (t, J=1.8 Hz, 4H); ¹³C NMR δ43.64, 58.51, 69.22, 70.34, 70.43, 70.72, 73.45, 84.57; IR 1338 cm⁻¹; MS 361 (M⁺), 317; HRMS m/e calc'd for C₁₆H₂₅O₇S: 361.1321, found 361.1324.

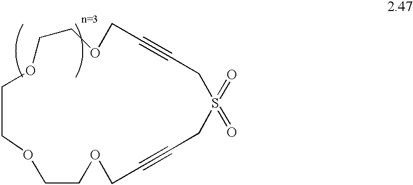

Compound 2.47: Following the general procedure (see compound 2.12), sulfide 2.32 (57.4 mg, 0.154 mmol) gave a residue after workup that was purified by flash chromatography on silica gel (10% MeOH in EtOAc) to afford sulfone 2.47 (60.4 mg, 97%) as colorless solid: m.p. 63-64° C.; R_{F} 0.44 (10% MeOH in EtOAc); ¹H NMR δ3.56-3.73 (m, 20H), 4.12 (t, J=2.0 Hz, 4H), 4.26 (t, J=2.0 Hz, 4H); ¹³C NMR δ43.63, 58.55, 69.66, 70.45, 70.71, 70.76, 70.82, 73.57, 84.66; IR 1337 cm⁻¹; MS 405 (MH⁺), 361, 341; HRMS m/e calc'd for C₁₈H₂₉O₈S: 405.1583, found 405.1593.

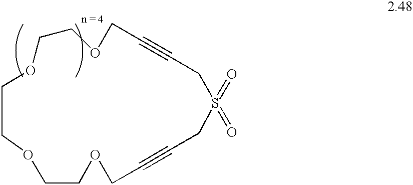

Compound 2.48: Following the general procedure (see compound 2.12), sulfide 2.33 (41.5 mg, 99.8 mmol) gave sulfone 2.48 (40.8 mg, 91%) after workup. An analytical sample recrystallized from 40% EtOAc in hexanes gave the following: m.p. 87-88° C.; R_{F} 0.32 (10% MeOH in EtOAc); ¹H NMR δ3.52-3.71 (m, 24H), 4.11 (s(br), 4H), 4.24 (s(br), 4H); ¹³C NMR δ43.56, 58.45, 69.17, 70.42, 70.60 (4C), 73.57, 84.53; IR 1355 cm⁻¹; MS 449 (MH⁺), 404, 384, 361; HRMS m/e calc'd for C₂₀H₃₃O₉S: 449.1845, found 449.1849.

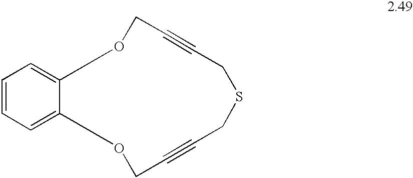

Compound 2.49:⁴ To a solution of dibromide 2.53 (170 mg, 0.481 mmol) in 48 mL of 5:1 CH₂Cl₂/EtOH was added Na₂S.Al₂O₃ (411 mg, 22% w/w Na₂S, 1.16 mmol Na₂S) in one portion. The reaction mixture was flushed well with argon and stirred at room temperature for 2 days. The heterogeneous reaction mixture was filtered through Celite, the solids were washed with EtOAc and the combined eluants were concentrated. The residue was purified by flash chromatography on silica gel (25% EtOAc in hexanes) to afford sulfide 2.49 (61.1 mg, 52%) as a colorless solid: m.p. 95-96° C.; R_{F} 0.48 (25% EtOAc in hexanes); ¹H NMR δ3.78 (t, J=2.3 Hz, 4H), 4.84 (t, J=2.3 Hz, 4H), 6.97 (s, 4H); ¹³C NMR δ21.84, 58.32, 77.94, 84.07, 118.33, 122.83 148.37; IR 2232, 1245, 994 cm⁻¹; MS 245 (MH⁺), 213, 161, 136; HRMS m/e calc'd for C₁₄H₁₂O₂S: 244.0558, found 244.0564.

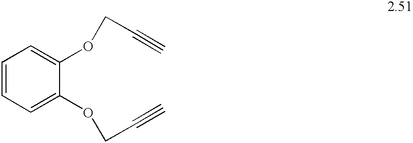

Compound 2.51:¹¹ A 50 mL round bottom flask containing an oval stir bar was charged with catechol (1.39 g, 12.6 mmol) and K₂CO₃ (3.48 g, 25.2 mmol) and the reaction vessel was flushed well with argon. An argon-flushed reflux condenser was attached and 15 mL of dry acetone and propargyl bromide (2.8 mL, 80% w/w, 25.2 mmol) were added. The reaction mixture was vigorously stirred and heated to reflux with the exclusion of light for 7.5 h and then allowed to cool to room temperature. The heterogeneous reaction mixture was filtered through Celite, the solids were washed with acetone, and the combined eluant was concentrated. The residue was resuspended in 75 mL of Et₂O and washed with 25 mL of 10% w/v aq. NaOH, 25 mL of water and dried (K₂CO₃). The organic layer was filtered, concentrated and resuspended in 120 mL of pentane. The suspension was heated to boiling and the mother liquor was decanted and concentrated to afford bis(propargyl ether) 2.51 (1.87 g, 80%) as a light golden oil. Preparative TLC (2 mm silica gel plate, CHCl₃) gave an analytically pure sample of compound 2.51 as a pale yellow solid: m.p. 32.5-33.5° C. (Lit. 30.5-31.0° C.¹¹); R_{F} 0.65 (CHCl₃); ¹H NMR δ2.49 (t, J=2.8 Hz, 2H), 4.72 (d, J=2.8 Hz, 4H), 6.91-7.0 (m, 2H), 7.0-7.09 (m, 2H); ¹³C NMR δ56.67, 75.68, 78.47, 114.93, 121.97, 147.41; IR 2130, 1251, 1035 cm⁻¹; MS 187 (MH⁺), 147, 131; HRMS m/e calc'd for C₁₂H₁₁O₂: 187.0759, found 187.0752.

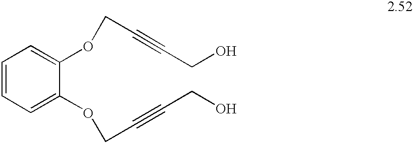

Compound 2.52: To a solution of bis(propargyl ether) 2.51 (152 mg, 0.818 mmol) and TMEDA (540 μL, 3.58 mmol) in 5.5 mL of THF under argon in a 10 mL round bottom flask and cooled to −78° C. via dry ice/i-PrOH bath was added n-BuLi (950 μL, 1.9 M, 1.8 mmol) dropwise over 2 min with stirring. After an additional 5 min, a stirring suspension of paraformaldehyde (367 mg, 12.27 mmol CH₂O equivalents) in 2.0 mL of THF under argon was added via 18-gauge cannula with vigorous stirring. The cooling bath was removed and the reaction mixture was allowed warm to room temperature with good stirring over the next 1 h 20 min. The yellow, heterogeneous reaction mixture was transferred to a separatory funnel and diluted with 50 mL of EtOAc and 30 mL of 25:5 saturated aq. NaH₂PO₄/water. The layers were mixed, allowed to separate and the aqueous layer was extracted with 3×15 mL of EtOAc. The combined organic layers were washed with 20 mL saturated aq. NaHCO₃ and 25 mL of brine. The residue upon drying and concentration of the organic layer was purified via preparative TLC (2 mm silica gel plate, 25% hexanes in EtOAc) to afford diol 2.52 (92.2 mg, 46%) as a light yellow solid: m.p. 91.5-92.5° C.; R_{F} 0.39 (25% hexanes in EtOAc); ¹H NMR (DMSO-d₆) δ4.10 (dt, J=5.7, 2.1 Hz, 4H), 4.81 (t, J=2.1 Hz, 4H), 5.24 (t, J=5.7 Hz, 2H), 6.88-6.97 (m, 2H), 6.97-7.06 (m, 2H); ¹³C NMR (MeOH-d₄) δ50.67, 57.92, 80.83, 87.04, 116.33, 123.07, 149.06; IR 1263, 1207, 1018 cm⁻¹; MS 247 (MH⁺), 229, 212, 161; HRMS m/e calc'd for C₁₄H₁₄O₄: 246.0892, found 246.0894.

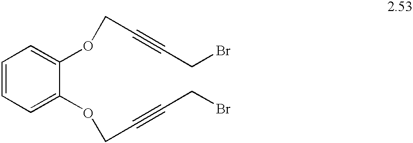

Compound 2.53: To an ice-water bath-cooled solution of PPh₃ (454 mg, 1.73 mmol) in 6 mL of 1:1 CH₂Cl₂/THF was added Br₂ (87 μL, 1.70 mmol) dropwise slowly via gastight syringe with good stirring followed 5 min later by pyridine (114 μL, 1.41 mmol). A solution of diol 2.52 (174 mg, 0.706 mmol) in 2.4 mL of 1:1 CH₂Cl₂/THF under argon was added slowly via cannula with good stirring and the reaction mixture was allowed to warm to room temperature as stirring was continued for an additional 10 h. The golden, heterogeneous reaction mixture was transferred to a separatory funnel and diluted with 40 mL of EtOAc and 35 mL of 4:1 saturated aq. NaHCO₃/water. The layers were mixed, allowed to separate, and the aqueous layer was extracted with 3×15 mL of EtOAc. The combined organic layers were washed with 35 mL of brine and dried. The residue upon concentration of the organic layer was purified by flash chromatography on silica gel (35% hexanes in EtOAc) to afford dibromide 2.53 (201 mg, 77%) as a pale yellow solid. An analytical sample recrystallized from 50% hexanes in EtOAc gave the following: m.p. 84.5-85.5° C.; R_{F} 0.77 (50% hexanes in EtOAc); ¹H NMR δ3.91 (t, J=2.2 Hz, 4H), 4.79 (t, J=2.2 Hz, 4H), 6.93-7.05 (m, 4H); ¹³C NMR δ13.97, 56.99, 81.56, 82.45, 114.79, 122.08, 147.4; IR 617 cm⁻¹; MS 371 (MH⁺), 291, 239, 223, 212, 173, 160, 144; HRMS m/e calc'd for C₁₄H₁₂Br₂O₂: 369.9204, found 369.92.

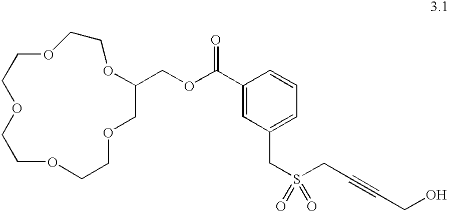

Compound 3.1: Following the general method (see compound 3.2), compound 3.15 (0.048 g, 0.103 mmol) afforded after workup sulfone 3.1 (0.04 g, 78%) as a colorless oil. Sulfone 3.1 exhibited partial isomerization to the allene when chromatographic purification involving silica gel (preparative silica gel TLC) was attempted. When necessary, compound 3.1 could be purified via C-18 derivatized silica gel, employing 60:40 MeOH/water as the eluant. Analytical data for sulfone 3.1: R_{F} 0.35 (10% MeOH in CHCl₃); ¹H NMR δ3.30 (s(br), 1H), 3.53-3.73 (m, 18H), 3.73-3.90 (m, 2H), 3.90-4.04 (m, 1H), 4.33 (dd, J=11.8, 6.5 Hz, 1H), 4.36 (t, J=2.2 Hz, 2H), 4.43 (dd, J=11.8, 4.7 Hz, 1H), 4.48 (s, 2H), 7.48 (t, J=8.0 Hz, 1H), 7.68 (d, J=8.0 Hz, 1H), 8.04 (d, J=8.0 Hz, 1H), 8.17 (s, 1H); ¹³C NMR δ43.48, 50.58, 56.85, 64.77, 70.10, 70.15, 70.32, 70.44, 70.62, 70.95, 72.55, 77.31, 88.00, 128.17, 129.33, 130.39, 130.79, 131.89, 135.37, 165.62; IR 3354, 1727, 1331 cm⁻¹; MS 501 (MH⁺), 369; HRMS m/e calc'd for C₂₃H₃₃O₁₀S: 501.1794, found 501.1802.

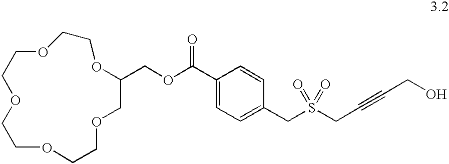

General procedure for sulfone formation. Compound 3.2: To an ice-water bath-cooled solution of sulfide 3.14 (0.014 g, 0.03 mmol) in 350 μL of MeOH was added dropwise with vigorous stirring a solution of Oxone® (49.5% w/w KHSO₅, 0.13 g, 0.105 mmol) in 350 μL of water and 150 μL of 2.5M, pH 5.5 potassium citrate buffer, and the resulting heterogeneous reaction mixture was allowed to stir overnight as the ice-water bath melted. The reaction mixture was diluted with 25 mL of water and this was extracted with CHCl₃ (3×15 mL). The combined organic extracts were washed with water (10 mL) and saturated aqueous KCl (15 mL). The residue upon drying (K₂SO₄) and concentration of the organic layer afforded sulfone 3.2 (0.014 g, 91%) as a pale pink oil. Sulfone 3.2 exhibited some isomerization to the allene after chromatographic purification (silica gel) was attempted via flash column or preparative TLC. When necessary compound 3.2 could be purified via C-18 derivatized silica gel, employing 60:40 MeOH/water as the eluant. Analytical data for sulfone 3.2: R_{F} 0.40 (10% MeOH in CHCl₃); ¹H NMR δ3.33 (s(br), 1H), 3.55-3.74 (m, 16H), 3.70 (s(br), 2H), 3.74-3.87 (m, 2H), 3.87-4.0 (m, 1H), 4.32 (dd, J=11.8, 6.5 Hz, 1H), 4.35 (s(br), 2H), 4.48 (dd, J=11.8, 4.7 Hz, 1H), 4.49 (s, 2H), 7.55 (d, J=9.2 Hz, 2H), 8.05 (d, J=8.6 Hz, 2H); ¹³C NMR δ43.84, 50.86, 57.19, 64.89, 70.16 (3C), 70.34 (2C), 70.43, 70.50, 70.75, 70.93, 72.78, 77.36, 87.34, 130.28, 130.97 (2C), 132.43, 165.72; IR 3332, 1722, 1329 cm⁻¹; MS 501 (MH⁺), 433, 369; HRMS m/e calc'd for C₂₃H₃₃O₁₀S: 501.1794, found 501.1786.

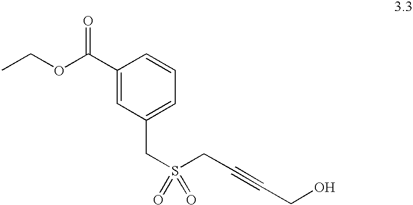

Compound 3.3: Following the general procedure (see compound 3.2), sulfide 3.16 (0.02 g, 0.076 mmol) gave a residue after workup that was purified by flash column chromatography on silica gel (1:1 EtOAc/hexanes) to afford sulfone 3.3 (0.018 g, 82%) as a colorless, crystalline solid: m.p. 79.5-80.5° C.; R_{F} 0.29 (1:1 EtOAc/hexanes); ¹H NMR δ1.39 (t, J=8.4 Hz, 3H), 2.44 (s(br), 1H), 3.67 (t, J=2.2 Hz, 2H), 4.36 (t, J=2.2 Hz, 2H), 4.37 (q, J=8.4 Hz, 2H), 4.49 (s, 2H), 7.50 (t, J=8.8 Hz, 1H), 7.69 (d, J=8.8 Hz, 1H), 8.05 (d, J=8.8 Hz, 1H), 8.18 (s, 1H); ¹³C NMR δ14.25, 43.77, 50.97, 57.35, 61.52, 73.08, 87.42, 127.93, 129.30, 130.26, 131.23, 132.25, 135.01, 166.11; IR 3494, 1718, 1316 cm⁻¹; MS 297 (MH⁺), 279, 251, 163; HRMS m/e calc'd for C₁₄H₁₇O₅S: 297.0797, found 297.0798.

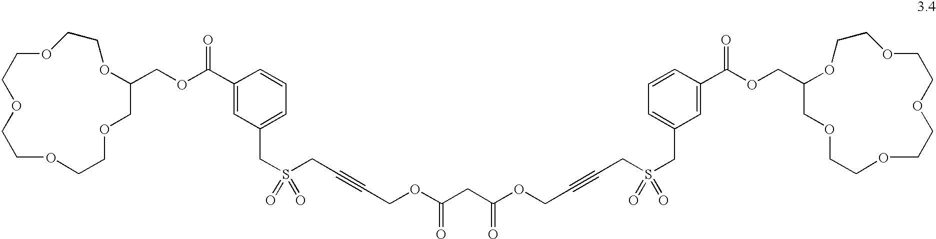

Compound 3.4: Following the general procedure (see Compound 3.2; requires 7 equivalents of oxidant to oxidize both sulfur atoms), compound 3.17 (0.017 g, 0.17 mmol) afforded after workup bis(sulfone) 3.4 (0.016 g, 93%) as a colorless oil: ¹H NMR δ3.54-3.75 (m, 32H), 3.58 (s, 2H), 3.71 (s(br), 4H), 3.75-3.88 (m, 4H), 3.88-4.02 (m, 2H), 4.32 (dd, J=11.8, 6.5 Hz, 2H), 4.46 (dd, J=11.8, 4.7 Hz, 2H), 4.48 (s(br), 4H), 4.83 (s, 4H), 7.48 (t, J=7.5 Hz, 2H), 7.67 (d, J=7.5 Hz, 2H), 8.05 (d, J=7.5 Hz, 2H), 8.11 (s, 2H); ¹³C NMR δ40.76, 43.52, 53.08, 57.02, 65.10, 70.31 (3C), 70.48 (2C), 70.61, 70.71, 70.78, 71.07, 75.00, 77.43, 82.08, 127.93, 127.93, 129.28, 130.35, 131.09, 131.98, 135.24, 165.45, 135.59; IR 1770, 1726, 1334 cm⁻¹; MS (FAB) 1069 (MH⁺); HRMS (FAB) m/e calc'd for C₄₉H₆₅O₂₂S₂: 1069.3409, found 1069.3396.

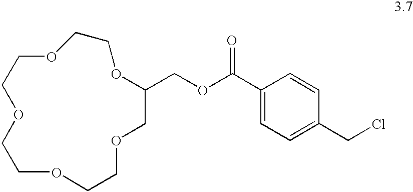

Compound 3.7: To an ice-water bath-cooled solution of t-BuOK (95% w/w, 0.103 g, 0.872 mmol) in 1 mL of THF was added quickly via cannula a solution of 2-(hydroxymethyl)-15-crown-5 (96% w/w, 0.217 g, 0.832 mmol) in 1 mL of THF. The resulting yellow alkoxide solution was allowed to stir an additional 1 min and was then added in portions over 3.5 min via cannula to an ice-water bath-cooled stirring suspension of 4-chloromethylbenzoyl chloride (97% w/w, 0.168 g, 0.862 mmol) and DMAP (0.106 g, 0.868 mmol) in 3 mL of THF. The cooling bath was removed and the reaction mixture was allowed to stir at room temperature overnight. The reaction mixture was diluted with 60 mL of EtOAc and 30 mL of 2:1 saturated aqueous KCl/water, the layers were mixed and then separated, and the aqueous layer was extracted with EtOAc (3×15 mL). The combined organic layers were washed with saturated aqueous KH₂PO₄ (2×15 mL), saturated aqueous KHCO₃ (20 mL), and saturated aqueous KCl (20 mL). The organic layer was dried (K₂SO₄), concentrated, and the residue was purified by flash column chromatography on silica gel (10% MeOH in CHCl₃) to afford ester 3.7 (0.151 g, 45%) as a light yellow oil: R_{F} 0.54 (10% MeOH in CHCl₃); ¹H NMR δ3.50-3.72 (m, 16H), 3.72-3.85 (m, 2H), 3.85-3.97 (m, 1H), 4.26 (dd, J=11.8, 6.5 Hz, 1H), 4.41 (dd, J=11.8, 4.7 Hz, 1H), 4.53 (s, 2H), 7.38 (d, J=9.4 Hz, 2H), 7.96 (d, J=9.4 Hz, 2H); ¹³C NMR δ45.24, 64.87, 70.29, 70.42 (3C), 70.44, 70.58, 70.66, 70.81, 71.01, 77.44, 128.38, 129.95 (2C), 142.21, 165.78; IR 1724, 722 cm⁻¹; MS 403 (MH⁺), 367; HRMS m/e calc'd for C₁₉H₂₈ClO₇: 403.1524, found 403.1516.

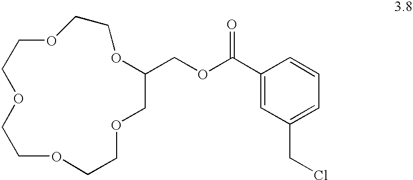

Compound 3.8: To a solution of 3-chloromethylbenzoyl chloride (98% w/w, 0.357 g, 1.85 mmol) in 1.5 mL of THF and 1.5 mL of CH₂Cl₂ was added dropwise with stirring via cannula a solution of 2-(hydroxymethyl)-15-crown-5 (96% w/w, 0.628 g, 2.41 mmol), DMAP (0.059 g, 0.483 mmol), and pyridine (150 μL , 1.86 mmol) in 1 mL of THF and 1 mL of CH₂Cl₂. The reaction mixture was heated under reflux for 13 h, allowed to cool to room temperature and transferred to a separatory funnel containing 50 mL of EtOAc and 25 mL of 15:5:5 saturated aqueous KCl/saturated aqueous KH₂PO₄/water. The layers were mixed and then separated, and the aqueous layer was extracted with EtOAc (3×20 mL). The combined organic layers were washed with aqueous saturated KCl (20 mL). The organic layer was dried (K₂SO₄), concentrated and the residue was purified by flash column chromatography on silica gel (10% MeOH in CHCl₃) to afford an oil that contained the desired ester contaminated with ˜20% of the acid derived from the acid chloride starting material. The oil was dissolved in 75 mL of EtOAc and was washed with saturated aqueous KHCO₃ (2×30 mL) and saturated aqueous KCl (30 mL). Drying (K₂SO₄) and concentration of the organic layer afforded ester 3.8 (0.476 g, 64%) as a light yellow oil: R_{F} 0.56 (10% MeOH in CHCl₃); ¹H NMR δ3.35-3.62 (m, 16H), 3.62-3.77 (m, 2H), 3.77-3.86 (m, 1H), 4.16 (dd, J=11.8, 6.5 Hz, 1H), 4.31 (dd, J=11.8, 4.7 Hz, 1H), 4.44 (s, 2H), 7.26 (t, J=7.7 Hz, 1H), 7.41 (d, J=7.7 Hz, 1H), 7.82 (d, J=7.7 Hz, 1H); ¹³C NMR δ44.99, 64.58, 69.89, 69.98, 70.06 (3C), 70.23, 70.39 (2C), 70.71, 77.00, 128.39, 129.02, 129.14, 130.19, 132.55, 137.41, 165.25; IR 1730, 714 cm⁻¹; MS 403 (MH⁺), 367; HRMS m/e calc'd for C₁₉H₂₈ClO₇: 403.1524, found 403.1519.

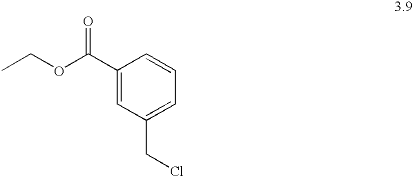

Compound 3.9:¹² To a solution of 3-chloromethylbenzoyl chloride (98% w/w, 0.502 g, 2.6 mmol) in 3 mL of THF was added dropwise via cannula with stirring a solution of EtOH (470 μL, 8.0 mmol), DMAP (0.066 g, 0.54 mmol), and pyridine (215 μL, 2.66 mmol) in 2 mL of THF. The reaction mixture was heated under reflux for 11 h, allowed to cool to room temperature, and transferred to a separatory funnel containing 50 mL of EtOAc and 25 mL of 15:5:5 brine/saturated aqueous NaH₂PO₄/water. The layers were mixed and then separated, and the aqueous layer was extracted with EtOAc (3×15 mL). The combined organic layers were washed with water (15 mL) and brine (15 mL). The organic layer was dried, concentrated, and the residue was purified by flash column chromatography on silica gel (20% EtOAc in hexanes) to afford ester 3.9 (0.426 g, 83%) as a colorless oil: R_{F} 0.59 (20% EtOAc in hexanes); ¹H NMR δ1.38 (t, J=8.6 Hz, 3H), 4.35 (q, J=8.6 Hz, 2H), 4.59 (s, 2H), 7.4 (t, J=9.6 Hz, 1H), 7.55 (d, J=9.6 Hz, 1H), 7.97 (d, J=9.6 Hz, 1H), 8.03 (s, 1H); ¹³C NMR δ14.21, 45.44, 61.04, 128.74, 129.4, 129.5, 130.94, 132.79, 137.71, 165.93; IR 1719, 723 cm⁻¹; MS 199 (MH⁺), 163; HRMS m/e calc'd for C₁₀H₁₂ClO₂: 199.0526, found 199.0528.

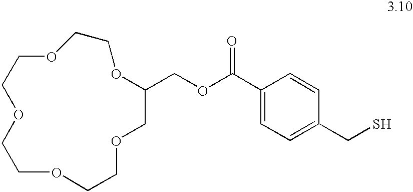

General procedure for thiol formation. Compound 3.10: A solution of compound 3.7 (0.221 g, 0.549 mmol) and thiourea (0.097 g, 1.27 mmol) in 3 mL of EtOH was heated with stirring under reflux for 21 h. The reaction mixture was then cooled with an ice-water bath, and n-butylamine (165 μL, 1.67 mmol) was added with stirring. The reaction mixture was allowed to stir for 2 h as the ice-water bath melted and warmed to room temperature. The reaction mixture was diluted with 30 mL of CHCl₃ and 25 mL of 15:5:5 saturated aqueous KCl/saturated aqueous KH₂PO₄/water, the layers were mixed, separated, and the aqueous layer was extracted with CHCl₃ (3×10 mL). The organic layer was dried (K₂SO₄), concentrated, and the residue was purified by preparative TLC (2 mm silica gel plate, 10% MeOH in CHCl₃) to afford two thiol 3.10-containing fractions (A and B) that were contaminated with the disulfide of compound 3.10 as determined by ¹H NMR (fraction A: 52 mg, 53.9% w/w 3.10; fraction B: 66 mg, 27.4% w/w 3.10; this represents an initial 54% conversion to thiol 3.10). Fraction A was further purified by preparative TLC (1 mm silica gel plate, 10% MeOH in CHCl₃) to afford thiol 3.10 as a light greenish-yellow oil: R_{F} 0.60 (10% MeOH in CHCl₃); ¹H NMR δ1.75 (t, J=7.9 Hz, 1H), 3.54-3.70 (m, 16H), 3.75 (d, J=7.9 Hz, 2H), 3.77-3.87 (m, 2H), 3.87-4.0 (m, 1H), 4.28 (dd, J=11.8, 6.5 Hz, 1H), 4.43 (dd, J=11.8, 4.7 Hz, 1H), 7.36 (d, J=9.1 Hz, 2H), 7.95 (d, J=7.5 Hz, 2H); ¹³C NMR δ28.65, 64.79, 70.37, 70.43, 70.52 (2C), 70.70 (2C), 70.79, 70.96, 71.10, 77.57, 128.00, 129.81, 130.03, 146.31, 166.02; IR 2555, 1722 cm⁻¹; MS 401 (MH⁺), 367; HRMS m/e calc'd for C₁₉H₂₉O₇S: 401.1634, found 401.1632.

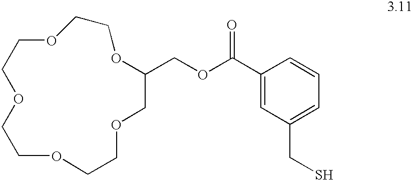

Compound 3.11: Following the general procedure (see compound 3.10), chloride 3.8 (0.45 g, 1.12 mmol) gave a residue after workup that was purified by flash column chromatography on silica gel (10% MeOH in CHCl₃) to afford the thiol 3.11 (0.277 g, 62%) as a light yellow oil: R_{F} 0.60 (10% MeOH in CHCl₃); ¹H NMR δ1.60-1.94 (s(br), 1H), 3.40-3.62 (m, 16H), 3.64 (s, 2H), 3.65-3.77 (m, 2H), 3.77-3.89 (m, 1H), 4.18 (dd, J=11.8, 6.5 Hz, 1H), 4.34 (dd, J=11.8, 4.7 Hz, 1H), 7.25 (t, J=9.7 Hz, 1H), 7.38 (d, J=7.6 Hz, 1H), 7.76 (d, J=7.6 Hz, 1H), 7.84 (s, 1H); ¹³C NMR δ28.14, 64.54, 69.98, 70.05, 70.15 (2C), 70.32 (2C), 70.44, 70.54, 70.76, 77.13, 127.85, 128.36, 128.76, 130.10, 132.29, 141.16, 165.65; IR 2550, 1724 cm⁻¹; MS 401 (MH⁺), 367; HRMS m/e calc'd for C₁₉H₂₉O₇S: 401.1634, found 401.1632.

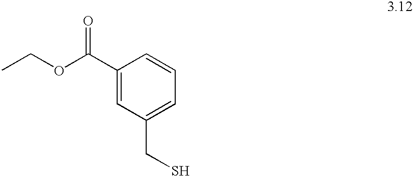

Compound 3.12: Following the general procedure (see compound 3.10), chloride 3.9 (0.229 g, 1.15 mmol) gave a residue after workup that was purified by flash column chromatography on silica gel (20% EtOAc in hexanes) to afford the thiol 3.12 ( 0.166 g, 73%) as a colorless oil: R_{F} 0.63 (20% EtOAc in hexanes); ¹H NMR δ1.35 (t, J=8.0 Hz, 3H), 1.76 (t, J=8.3 Hz, 1H), 3.72 (d, J=8.3 Hz, 2H), 4.33 (q, J=8.0 Hz, 2H), 7.34 (t, J=8.5 Hz, 1H), 7.47 (d, J=7.47 Hz, 1H), 7.87 (d, J=8.5 Hz, 1H), 7.95 (s, 1H); ¹³C NMR δ14.17, 28.46, 60.86, 128.06, 128.56, 128.90, 130.73, 132.33, 141.34, 166.10; IR 2574, 1721 cm⁻¹; MS 197 (MH⁺), 163; HRMS m/e calc'd for C₁₀H₁₃O₂S: 197.0636, found 197.0633.

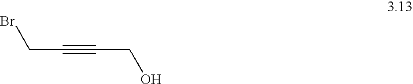

Compound 3.13:¹³ To an ice-water bath-cooled solution of triphenylphosphine (3.2 g, 12.2 mmol) in 65 mL of THF was added bromine (600 μL, 11.7 mmol) dropwise via gastight syringe with stirring. The reaction mixture was allowed to stir for 5 min and then a solution of 2-butyn-1,4-diol (1.0 g, 11.6 mmol) in 7 mL of THF was added quickly via cannula with vigorous stirring. Stirring was continued at 0° C. for 0.5 h and then at room temperature for 4 h. The reaction mixture was transferred to a separatory funnel, shaken with 50 mL of water and 20 mL of brine, the layers were separated, and the aqueous layer was extracted with EtOAc (3×25 mL). The combined organic layers were washed with saturated aqueous NaHSO₃ (20 mL), saturated aqueous NaHCO₃ (20 mL) and brine (25 mL). The organic layer was dried (Na₂SO₄) and concentrated to a residue which was purified by flash column chromatography on silica gel (40% EtOAc in hexanes). Early eluting fractions were pooled to yield bromide 3.13 (0.542 g, 31%) as a light yellow oil: R_{F} 0.48 (3:2 hexanes/EtOAc); ¹H NMR δ1.78 (s(br), 1H), 3.94 (t, J=2.0 Hz, 2H), 4.32 (t, J=2.0 Hz, 2H); ¹³C NMR δ14.20 (50.99, 80.73, 84.86); MS 149 (MH⁺), 131; HRMS m/e calc'd for C₄H₆BrO: 148.9602, found 148.9601.

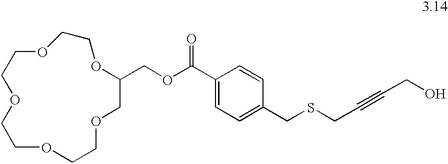

General procedure for sulfide formation. Compound 3.14: To a solution of thiol 3.10 (0.03 g, 0.075 mmol) in 600 μL of EtOH was added 4-bromobut-2-ynol (15 μL of a 1.13 mg/μL solution in EtOH, 0.144 mmol) with stirring and cooling via ice-water bath. Hünig's base (16 μL, 0.092 mmol) was added and, after 5 minutes, the cooling bath was removed and the reaction mixture was stirred an additional 21 h at room temperature. The reaction mixture was diluted with 25 mL of EtOAc and 25 mL of 15:5:5 saturated aqueous KCl/saturated aqueous KH₂PO₄/water, the layers were mixed, separated, and the aqueous layer was extracted with EtOAc (3×15 mL). The combined organic layers were washed with water (10 mL) and saturated aqueous KCl (15 mL). The organic layer was dried (K₂SO₄), concentrated, and the residue was purified by preparative TLC (1 mm silica gel plate, 10% MeOH in CHCl₃) to afford sulfide 3.14 (0.032 g, 90%) as a light red oil.

From chloride 3.7. To a 5 mL round bottom flask containing a suspension of thiourea (28.1 mg, 0.37 mmol) in 400 μL of EtOH was added bromide 3.13 (45 μL of a 1.13 mg/μL solution in EtOH, 0.343 mmol) with stirring. An argon-flushed reflux condenser equipped with a Teflon sleeve over the male joint was attached to the reaction vessel, and the reaction mixture was heated for 14 h at 48° C. and then allowed to cool to room temperature. The reflux condenser was replaced with a septum, and an additional 100 μL of EtOH was added. The reaction mixture was cooled to 0° C. via ice-water bath. n-BuNH₂ (34 μL, 0.343 mmol) was added with stirring as the reaction mixture was allowed to warm to room temperature over 45 min. A solution of chloride 3.7 (0.138 g, 0.343 mmol) in 400 μL of EtOH was added slowly via cannula with stirring followed by Hünig's base (60 μL, 0.343 mmol). The reaction mixture was allowed to stir at room temperature for an additional 22 h and was then transferred to a separatory funnel containing 70 mL of EtOAc and 62 mL of 42:10:10 saturated aqueous KCl/saturated aqueous KH₂PO₄/water. The layers were mixed, allowed to separate, and the aqueous layer was extracted with 2×40 mL of EtOAc. The combined organic layers were washed with 15 mL of water and 25 mL of saturated aqueous KCl and dried. The residue upon concentration of the organic layer was purified by preparative TLC (1 mm silica gel plate, 10% MeOH in CHCl₃) to afford two sulfide 3.14-containing fractions, A and B: fraction A (lower R_{F}, 75.5 mg, pale yellow oil) contained pure sulfide 3.14; fraction B (36.9 mg) contained 15% w/w sulfide 3.14 contaminated with compound 3.7. The total yield of compound 3.14 based on recovered 3.7 was 65%. Analytical data for sulfide 3.14: R_{F} 0.52 (10% MeOH in CHCl₃); ¹H NMR δ2.25 (s(br), 1H), 3.08 (t, J=2.2 Hz, 2H), 3.52-3.70 (m, 16H), 3.70-3.87 (m, 2H), 3.84 (s, 2H), 3.87-4.0 (m, 1H), 4.27 (dd, J=11.8, 6.5 Hz, 1H), 4.27 (s(br), 2H), 4.44 (dd, J=11.8, 4.7 Hz, 1H), 7.36 (d, J=8.7 Hz, 2H), 7.95 (d, J=8.0 Hz, 2H); ¹³C NMR δ18.85, 35.16, 51.05, 64.77, 70.36, 70.43 (2C), 70.51 (2C), 70.70, 70.79, 70.96, 71.10, 77.58, 81.02, 81.79, 128.79, 128.94, 129.81, 143.00, 166.10; IR 3576, 1725 cm⁻¹; MS 469 (MH⁺), 451, 401; HRMS m/e calc'd for C₂₃H₃₃O₈S: 469.1896, found 469.1904.

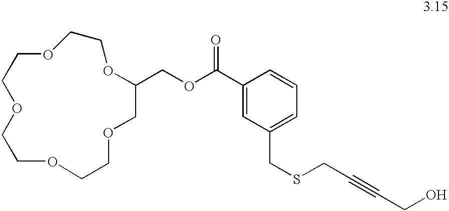

Compound 3.15: Following the general procedure (see compound 3.14), thiol 3.11 (0.051 g, 0.128 mmol) gave a residue after workup that was purified by preparative TLC (1 mm silica gel plate, 10% MeOH in CHCl₃) to afford sulfide 3.15 (0.049 g, 83%) as a light pink oil: R_{F} 0.50 (10% MeOH in CHCl₃); ¹H NMR δ2.9 (s(br), 1H), 3.06 (t, J=2.2 Hz, 2H), 3.51-3.75 (m, 16H), 3.75-3.91 (m, 2H), 3.86 (s, 2H), 3.91-4.02 (m, 1H), 4.28 (dd, J=11.8, 6.5 Hz, 1H), 4.28 (s(br), 2H), 4.41 (dd, J=11.8, 4.7 Hz, 1H), 7.37 (t, J=8.4 Hz, 1H), 7.52 (d, J=8.4 Hz, 1H), 7.90 (d, J=8.4 Hz, 1H), 8.0 (s, 1H); ¹³C NMR δ18.61, 34.70, 50.63, 64.52, 70.04, 70.11, 70.18, 70.27, 70.34, 70.43, 70.55, 70.71, 70.86, 77.33, 80.38, 82.45, 128.30, 128.60, 129.96, 130.13, 133.49, 138.02, 166.13; IR 3361, 1727 cm⁻¹; MS 469 (MH+), 403; HRMS m/e calc'd for C₂₃H₃₃O₈S: 469.1896, found 469.1899.

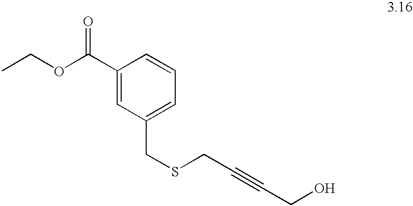

Compound 3.16: Following the general procedure (see compound 3.14), thiol 3.12 (0.097 g, 0.495 mmol) gave a residue after workup that was purified by flash column chromatography on silica gel (1:1 EtOAc/hexanes) to afford sulfide 3.16 (0.110 g, 84%) as a pale yellow oil: R_{F} 0.55 (1:1 EtOAc/hexanes); ¹H NMR δ1.38 (t, J=8.0 Hz, 3H), 2.13 (s(br), 1H), 3.09 (t, J=2.2 Hz, 2H), 3.88 (s, 2H), 4.30 (t, J=2.2 Hz, 2H), 4.36 (q, J=8.0 Hz, 2H), 7.37 (t, J=8.0 Hz, 1H), 7.52 (d, J=8.0 Hz, 1H), 7.90 (d, J=8.0 Hz, 1H), 8.03 (s, 1H); ¹³C NMR δ14.27, 18.91, 35.15, 51.15, 61.14, 81.28, 81.90, 128.30, 128.59, 130.29, 130.62, 133.39, 138.00, 166.56; IR 3443, 1716 cm⁻¹; MS 265 (MH⁺), 247, 219; HRMS m/e calc'd for C₁₄H₁₇O₃S: 265.0898, found 265.0896.

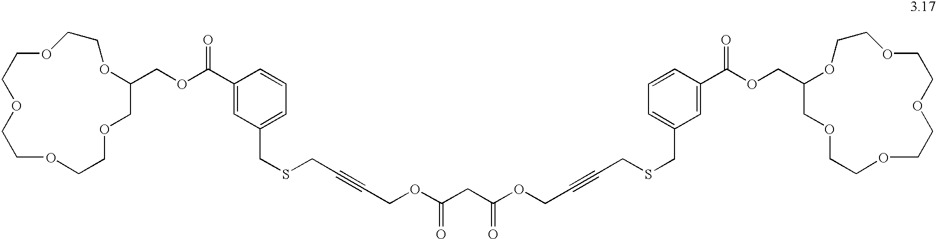

Compound 3.17: A stirring solution of sulfide 3.15 (0.139 g, 0.297 mmol), DMAP (0.018 g, 0.147 mmol) and Hünig's base (48 μL, 0.276 mmol) in 1.1 mL of THF was cooled with an ice-water bath and malonyl dichloride (97% w/w, 16 μL, 0.16 mmol) was added dropwise. After 10 min the cooling bath was removed and the reaction mixture was heated under reflux for 13 h. Upon cooling to room temperature, the reaction mixture was diluted with 75 mL of EtOAc and 50 mL of 30:10:10 saturated aqueous KCl/saturated aqueous KH₂PO₄/water. The layers were mixed, separated, and the aqueous layer was extracted with EtOAc (3×30 mL). The combined organic layers were washed with water (20 mL), saturated aqueous KHCO₃ (2×20 mL), and saturated aqueous KCl (35 mL). The organic layer was dried (K₂SO₄), concentrated, and the residue was purified by preparative TLC (2 mm silica gel plate, 10% MeOH in CHCl₃) to give two product-containing fractions that were each resubjected to purification via preparative TLC (1 mm silica gel plate, 10% MeOH in EtOAc). The subsequent product-containing fractions were pooled to afford bis(sulfide) 3.17 (0.029 g, 26% based on recovered 3.15 [0.035 g]) as a colorless oil: R_{F} 0.52; 0.07 (10% MeOH in CHCl₃; 10% MeOH in EtOAc); ¹H NMR δ3.08 (t, J=2.2 Hz, 4H), 3.50 (s, 2H), 3.55-3.70 (m, 3H), 3.70-3.87 (m, 4H), 3.85 (s, 4H), 3.87-4.0 (m, 2H), 4.29 (dd, J=11.8, 6.5 Hz, 2H), 4.44 (dd, J=11.8, 4.7 Hz, 2H), 4.77 (t, J=2.2 Hz, 4H), 7.37 (t, J=8.0 Hz, 2H), 7.50 (d, J=8.0 Hz, 2H), 7.90 (d, J=8.0 Hz, 2H), 7.97 (s, 2H); ¹³C NMR δ18.71, 35.06, 40.89, 53.48, 64.83, 70.34, 70.42, 70.50, 70.53, 70.68 (2C), 70.79, 70.95, 71.09, 76.63, 77.53, 83.10, 128.45, 128.60, 130.11, 130.45, 133.54, 137.91, 165.44, 166.10; IR 1770, 1726 cm⁻¹; MS (FAB) 1005 (MH⁺); HRMS (FAB) m/e calc'd for C₄₉H₆₅O₁₈S₂: 1005.3612, found 1005.3603.

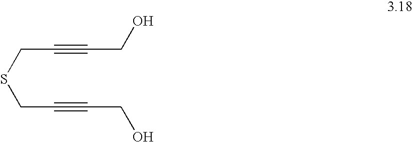

Compound 3.18:¹⁴ To a solution of chloride 2.14a (0.196 g, 1.88 mmol) in 45 mL of 5:1 CH₂Cl₂/EtOH was added Na₂S.Al₂O₃ (21% w/w Na₂S, 0.5 g, 1.35 mmol) in one portion with stirring. The reaction mixture was blanketed with argon and stirred at room temperature for 2 weeks. The heterogeneous reaction mixture was then filtered through Celite, the reaction solids were washed with CH₂Cl₂, and the combined eluants were concentrated in vacuo. The residue was purified by flash chromatography on silica gel (10% hexanes in EtOAc) to afford sulfide 3.18 (39.3 mg, 25%) as a pale yellow crystalline solid. An analytical sample prepared via preparative TLC (1 mm silica gel plate, 15% hexanes in EtOAc) gave a white crystalline solid with the following: m.p. 62.5-63.0° C. (Lit. 62° C.¹⁴); R_{F} 0.67 (10% hexanes in EtOAc); ¹H NMR δ1.82 (t, J=6.6 Hz, 2H), 3.45 (t, J=2.2 Hz, 4H), 4.29 (dt, J=6.6, 2.2 Hz, 4H); ¹³C NMR δ19.67, 50.93, 80.98, 81.51; MS 171 (MH⁺), 153, 135; HRMS m/e calc'd for C₈H₁₁O₂S: 171.0480, found 171.0482.

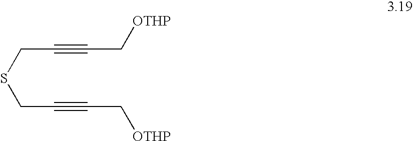

Compound 3.19: To a solution of bromide 2.14c (0.594 g, 2.6 mmol) in 1.7 mL of CH₂Cl₂ and 0.3 mL of EtOH was added Na₂S.Al₂O₃ (21% w/w Na₂S, 0.714 g, 1.9 mmol) in one portion and the reaction mixture was allowed to stir overnight at room temperature. The reaction mixture was filtered through Celite, the solids were washed with CH₂Cl₂, and the solvent was evaporated. The residue was purified by flash column chromatography on silica gel (20% EtOAc in hexanes) to afford sulfide 3.19 ( 0.182 g, 42%) as a pale yellow oil: R_{F} 0.34 (20% EtOAc in hexanes); ¹H NMR δ1.42-1.87 (m, 12H), 3.40 (t, J=2.1 Hz, 4H), 3.46-3.56 (m, 2H), 3.74-3.86 (m, 2H), 4.24 (dt, J=16.3, 2.1 Hz, 2H), 4.33 (dt, J=16.3, 2.1 Hz, 2H), 4.76 (t(br), J=3.7 Hz, 2H); ¹³C NMR δ18.95, 19.37, 25.24, 30.14, 54.39, 61.88, 79.07, 81.05, 96.69; IR 1120, 1033 cm⁻¹; MS 339 (MH⁺), 253, 237, 152; HRMS m/e calc'd for C₁₈H₂₇O₄S: 339.1630, found 339.1634.

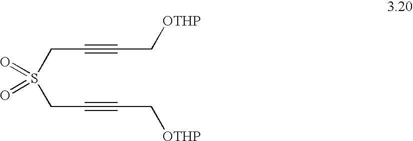

Compound 3.20: To an ice-water bath-cooled solution of sulfide 3.19 (0.055 g, 0.16 mmol) in 4 mL of CH₂Cl₂ was added m-CPBA (50% w/w, 0.158 g, 0.46 mmol) in one portion and the reaction mixture was allowed to stir at 0° C. for 0.5 h and then at room temperature for 5.5 h. The reaction mixture was diluted with EtOAc (50 mL), washed with saturated aqueous Na₂SO₃ (2×15 mL), saturated aqueous NaHCO₃ (2×15 mL) and brine (20 mL) and dried. The solvent was evaporated and the residue was purified by flash column chromatography on silica gel (1:1 EtOAc in hexanes) to afford sulfone 3.20 (0.027 g, 45%) as a colorless solid: m.p. 47-49° C.; R_{F} 0.57 (1:1 EtOAc/hexanes); 1H NMR δ1.42-1.87 (m, 12H), 3.46-3.53 (m, 2H), 3.74-3.83 (m, 2H), 4.07 (t, J=2.1 Hz, 4H), 4.24 (dt, J=15.9, 2.1 Hz, 2H), 4.33 (dt, J=15.9, 2.1 Hz, 2H), 4.76 (t(br), 2H); ¹³C NMR δ18.96, 25.25, 30.15, 43.63, 54.21, 62.09, 72.78, 84.68, 97.18; IR 1342, 1133, 1035 cm⁻¹; MS 371 (MH⁺), 285, 269; HRMS m/e calc'd for C₁₈H₂₇O₆S: 371.1528, found 371.1509.

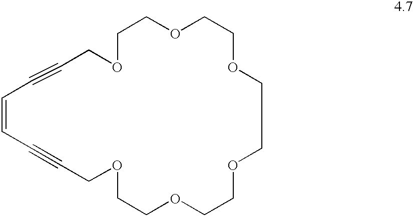

Compound 4.7.⁸ From α-chlorosulfone 4.22. To a solution of 4.22 (0.085 g, 0.199 mmol) in 6 mL of THF that had been cooled to −78° C. in a dry ice/acetone bath was added in one portion via cannula with stirring a dry ice/acetone bath-cooled suspension of t-BuOK (95% w/w, 0.05 g, 0.423 mmol) in 1 mL of THF. The dark brown reaction mixture was stirred at −78° C. for 15 minutes. Solid NaHCO₃ was added, followed by 10 mL of benzene. The reaction mixture was allowed to warm to room temperature and heated in an oil bath for 3 minutes at 50° C. The reaction mixture was then transferred to a separatory funnel containing 20 mL of brine and 20 mL of EtOAc. The layers were mixed and separated, and the aqueous layer was extracted with 3×30 mL of EtOAc. The residue upon drying and concentration of the organic layer was purified by flash column chromatography on silica gel (5% MeOH in EtOAc) to afford enediyne crown ether 4.7 (0.01 g, 11%) as a colorless oil that was 85% pure by HPLC (4.5×250 mm microsorb SiO₂, 1.0 mL/min EtOAc, t_{R}=9.2 min).

From dibromide 2.44. A vigorously stirred suspension of 2.44 (0.09 g, 0.18 mmol) and pulverized, desiccated NaBr (0.927 g, 9.0 mmol) in 15 mL of THF was cooled to −55° C. via dry ice/acetone bath. A room temperature solution of LiHMDS/TMEDA, formed by the addition of n-BuLi (2.2M, 410 μL, 0.902 mmol) to ice-water cooled solution of HMDS (190 μL, 0.9 mmol) and TMEDA (550 μL, 3.64 mmol) in THF (3 mL), was added dropwise over 4 min via addition funnel to the reaction mixture. The resulting dark green reaction mixture was allowed to stir an additional 22 minutes at −55° C. before being quenched with 10 mL of 2% aqueous HCl. The mixture was allowed to warm to room temperature and was transferred to a separatory funnel containing 12 mL of EtOAc. The aqueous layer was extracted with 3×10 mL EtOAc and the combined organic layers were washed with 3×5 mL of water and 8 mL of brine. The residue upon drying and concentration of the organic layer was purified by flash column chromatography in the dark using silica gel impregnated with 20% w/w AgNO₃ (15% MeOH in EtOAc). The eluant was collected into tubes containing 1 mL of brine. The organic layers in the fractions of interest were combined and evaporated to afford enediyne crown ether 4.7 (0.018 g, 29%) as a colorless oil: ¹H NMR δ3.55-3.73 (m, 20H), 4.34 (s, 4H), 5.79 (s, 4H); ¹³C NMR δ58.92, 68.88, 70.33, 70.57, 70.64, 70.71, 83.54, 92.87, 119.53; IR 3049, 2211, 1106 cm⁻¹; MS 339 (MH⁺); HRMS m/e calc'd for C₁₈H₂₆O₆: 339.1808, found 339.1809.

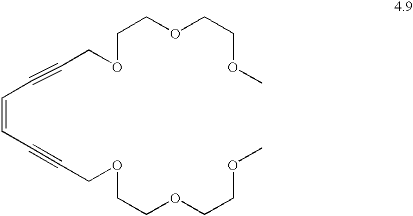

Compound 4.9: A 50 mL round bottom flask containing cuprous iodide (0.116 g, 0.61 mmol) and compound 4.40) (0.769 g, 4.87 mmol) under argon was charged with 15 mL of benzene, cis-1,2-ethylene dichloride (130 μL, 1.72 mmol), and n-BuNH₂ (1.55 mL, 15.7 mmol). Oxygen was removed by subjecting the mixture to three freeze-pump-thaw cycles. While still cool, a solution of Pd(PPh₃)₄ (80.7 mg, 69.8 μmol) in 1.0 mL of benzene under argon was added via cannula over 30 sec with stirring to the reaction mixture. The reaction vessel was covered with foil to exclude light and stirred at room temperature for 16.5 h. The reaction mixture was then filtered through a 20 g plug of silica gel and the plug was washed with 1:1 Et₂O/hexanes (150 mL), followed by EtOAc (150 mL). The EtOAc eluant was concentrated, and the residue was purified by flash chromatography on silica gel (5% hexanes in Et₂O) to afford a golden-brown oil which contained enediyne podand 4.9 (87% w/w, 0.421 g, 63%) contaminated with the Glaser coupling product of compound 4.40. An analytical sample of compound 4.9 prepared via preparative TLC (1 mm silica gel plate, 10% hexanes in Et₂O) gave a light yellow oil with the following: R_{F} 0.19 (5% hexanes in Et₂O); ¹H NMR δ3.34 (s, 6H), 3.47-3.55 (m, 4H), 3.57-3.66 (m, 8H), 3.66-3.73 (m, 4H), 4.35 (s, 4H), 5.81 (s, 2H); ¹³C NMR δ58.94, 59.03, 68.98, 70.37, 70.47, 71.83, 83.46, 92.84, 119.31; IR 3052, 1104 cm⁻¹; MS 341 (MH⁺), 237, 221; HRMS m/e calc'd for C₁₈H₂₉O₆: 341.1964, found 341.1966.

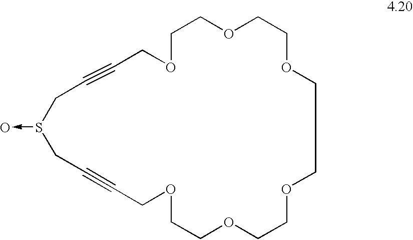

Compound 4.20:⁸ To a solution of sulfide 2.32 (0.062 g, 0.165 mmol) in 6.75 mL of CH₂Cl₂ that had been cooled to −30° C. via dry ice/i-PrOH bath was added dropwise with stirring via cannula over 30 seconds a solution of m-CPBA (0.063 g, 0.182 mmol) in 2.25 mL of CH₂Cl₂ and the resultant solution was allowed to stir at −30° C. for 1.25 h. While still cold, the reaction mixture was diluted with 20 mL of CH₂Cl₂ and then washed with 20 mL of saturated aqueous Na₂CO₃ and 20 mL of saturated aqueous NaHCO₃. The aqueous washes were extracted with 2×12 mL of CH₂Cl₂, and the combined organic layers were washed with 20 mL of brine. The residue upon drying and concentration of the organic layer was purified by flash column chromatography on silica gel (15% MeOH in EtOAc) to afford sulfoxide 4.20 (0.048 g, 75%) as a colorless solid: m.p. 50.5-51.5° C.; ¹H NMR δ3.53-3.63 (m, 20H), 3.68 (dt, J=15.9, 2.3 Hz, 2H), 3.83 (dt, J=15.9, 2.3 Hz, 2H), 4.19 (t, J=2.3 Hz, 4H); ¹³C NMR δ40.99, 58.48, 68.98, 70.27, 70.52, 70.58, 70.64, 74.13, 84.60; IR 2242, 2119, 1135, 1095, 1065 cm⁻¹; MS 389 (MH⁺), 337, 321; HRMS m/e calc'd for C₁₈H₂₉O₇S: 389.1634, found 389.1620.

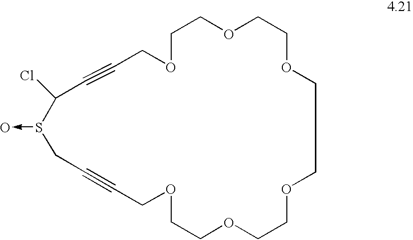

Compound 4.21:⁸ To a stirring solution of sulfoxide 4.20 (0.367 g, 0.946 mmol) in 20 mL of CH₂Cl₂ was added pyridine (170 μL, 2.1 mmol). The resulting solution was cooled to −78° C. in a dry ice/acetone bath. SO₂Cl₂ (160 μL, 1.98 mmol) was added in one portion with efficient stirring, and the reaction mixture was allowed to stir an additional 20 minutes at −78° C. The reaction mixture was then diluted with 40 mL of EtOAc. This solution was transferred to a separatory funnel containing 40 mL of EtOAc and 40 mL of water, the layers were mixed, allowed to separate, and the aqueous layer was extracted with 3×40 mL of EtOAc. The combined organic layers were washed with 20 mL of saturated aqueous NaHCO₃ and 15 mL of brine. Drying and concentration of the organic layer afforded chlorosulfoxide 4.21 (0.383 g, 96%) as a yellow oil that turns olive on standing. Chlorosulfoxide 4.21 could not be obtained analytically pure due instability to silica gel column chromatography but was sufficiently pure for immediate subsequent use: ¹H NMR (2:1 diastereomeric mixture) δ3.57-3.76 (m, 20H), 3.82-3.97 (m, 2H), 4.26 (t, J=1.6 Hz, 2H), 4.34 (d, J=1.6 Hz, 1.34H), 4.36 (d, J=1.6 Hz, 0.66H), 5.53 (t, J=1.6 Hz, 0.67H), 5.60 (t, J=1.6 Hz, 0.33H); ¹³C NMR δ41.24, 58.42, 58.45, 58.52, 61.13, 61.56, 69.10, 69.38, 70.29, 70.35, 70.60 (2C), 73.85, 73.95, 76.32, 85.12, 85.28, 89.95; IR 2231, 1110 cm⁻¹; MS 423 (MH⁺), 341, 305; HRMS m/e calc'd for C₁₈H₂₈ClO₇S: 423.1244, found 423.1238.

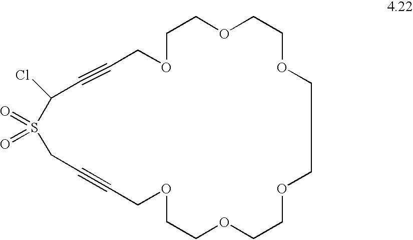

Compound 4.22:⁸ A solution of chlorosulfoxide 4.21 (0.222 g, 0.525 mmol) in 13 mL of CH₂Cl₂ was cooled to 0° C. via ice-water bath and peracetic acid (33% w/w, 0.61 g, 2.65 mmol) was added dropwise with stirring over 1 minute. The reaction mixture was allowed to stir at 0° C. for 0.5 h and then overnight at room temperature. The reaction mixture was diluted with 80 mL of EtOAc and transferred to a separatory funnel containing 30 mL of water. The layers were mixed and allowed to separate. The aqueous layer was extracted with 3×20 mL of EtOAc. The combined organic layers were washed with 40 mL of saturated aqueous Na₂SO₃ and 20 mL of brine. Drying and concentration afforded chlorosulfone 4.22 (0.195 g, 85%) as a pale yellow oil. Chlorosulfone 4.22 was unstable to silica gel column chromatography but was sufficiently pure for further use: ¹H NMR δ3.59-3.75 (m, 20H), 4.27 (s(br), 4H), 4.35 (d, J=1.7 Hz, 2H), 5.81 (t, J=1.7 Hz, 1H); ¹³C NMR δ41.99, 58.30, 59.78, 69.06, 69.34, 70.23, 70.32, 70.49 (2C), 70.52 (3C), 70.55 (2C), 72.36, 74.50, 85.37, 90.05; IR 2235, 1356 cm⁻¹; MS 439 (MH⁺), 405, 351; HRMS m/e calc'd for C₁₈H₂₈ClO₈S: 439.1193, found 439.1187.

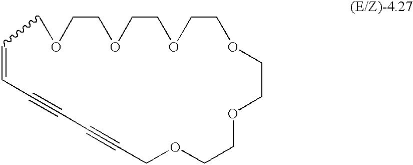

Compounds (E/Z)-4.27:⁸ DMPU (3.1 mL, 25.6 mmol) was added to a stirring solution of dibromide 2.44 (0.642 g, 1.28 mmol) in 107 mL of THF in a 500 mL flask equipped with a pressure-equalized addition funnel. After the reaction mixture was cooled to −63° C. in a dry ice/acetone bath, the addition funnel was charged with a room-temperature solution of LiHMDS that was prepared by the addition of n-BuLi (2.33 M, 1.25 mL, 2.91 mmol) to an ice-water bath-cooled solution of HMDS (620 μL, 2.94 mmol) in 21 mL of THF. The LiHMDS solution was added dropwise with vigorous stirring over 29 min. The reaction mixture was allowed to stir for an additional 16 min at −63° C. and was then quenched while still cold with 36 mL of 1% w/v aqueous HCl. The forest-green reaction mixture was allowed to warm to room temperature and was shaken with 85 mL of water and 100 mL of EtOAc in a separatory funnel. The aqueous layer was extracted with 4×50 mL of EtOAc, and the combined organic layers were washed with 60 mL of brine. The residue upon drying and concentration of the organic layer was purified by flash column chromatography on silica gel (5% MeOH in EtOAc) to remove the DMPU. All enediyne-containing fractions were pooled, concentrated and subjected to flash column chromatography in the dark on silica gel impregnated with 25% w/w AgNO₃ (15% MeOH in EtOAc) with the eluting fractions being collected into tubes containing 2 mL of brine. Separation of the organic layers of the fractions containing only one component afforded 22 mg of pure (Z)-4.27. Further purification of the earliest eluting mixed fractions [preparative TLC (1 mm silica gel plate, 5% MeOH in EtOAc), flash chromatography (silica gel, 5% MeOH in EtOAc), followed by preparative TLC (1 mm silica gel plate, EtOAc)] afforded an additional 14 mg of (Z)-4.27 as a pale yellow solid (36 mg total, 8%) and 2 mg of the slightly faster eluting (E)-4.27 contaminated with ˜5% of (Z)-4.27, as a colorless oil (0.5%). Analytical data for (Z)-4.27: mp 51-52° C.; ¹H NMR (d₆-DMSO) δ3.45 (m, 20H), 4.22 (d, J=6.9 Hz, 2H), 5.86 (d, J=11.3 Hz, 1H), 6.31 (dt, J=11.3, 6.9 Hz, 1H); ¹³C NMR (d₆-DMSO) δ58.16, 67.85) 68.78, 69.0, 69.60, 69.74, 69.78, 69.81, 69.90, 69.95, 74.50, 77.89, 81.46, 110.10, 144.84; MS 339 (MH⁺); HRMS m/e calc'd for C₁₈H₂₇O₆: 339.1808, found 339.1801.

Analytical data for (E)-4.27: ¹H NMR δ3.53 (m, 20H), 4.11 (dd, J=4.3, 1.9 Hz, 2H), 4.26 (s, 2H), 6.0 (d, J=15.9 Hz, 1H), 6.32 (dt, J=15.9, 4.3 Hz, 1H); MS 339 (MH⁺); HRMS m/e calc'd for C₁₈H₂₆O₆: 338.1729, found 338.1727.

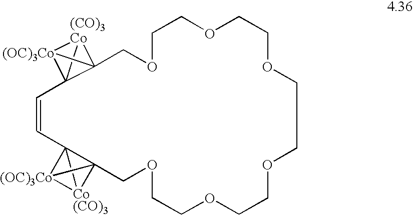

Compound 4.36: To a mixture of compounds 4.7 and (E/Z)-4.27 (41.6 mg, 61% w/w 4.7, 75 μmol 4.7) was added 3 mL of dry THF with stirring and cooling to 0° C. in an ice-water bath. An ice-water bath-cooled solution of Co₂(CO)₈ (0.258 g, 0.72 mmol) in 2 mL of THF under argon was added dropwise with stirring over 30 sec. The resultant dark red reaction mixture was allowed to stir at 0° C. for 15 min and then at room temperature for 1 h 15 min. The reaction mixture was concentrated to ˜0.5 mL with an air stream. The concentrated solution was purified via preparative TLC (1 mm silica gel plate, 25% hexanes in EtOAc) to afford compound 4.36 (23.1 mg, 61%) as a greenish-black solid that decomposed on standing at room temperature over 3 days: ¹H NMR δ3.6-3.73 (m, 16H), 3.83-3.88 (m, 4H), 4.86 (s, 4H), 6.38 (s, 2H); MS 910 (MH⁺), 882, 854, 825, 798, 770, 743, 715, 625.

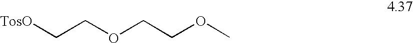

Compound 4.37:¹⁵ To a solution of pyridine (710 μL, 8.82 mmol) and diethylene glycol monomethyl ether (1.0 g, 8.32 mmol) in 9 mL of CH₂Cl₂ that had been cooled to 0° C. in an ice-water bath was added a suspension of tosyl chloride (1.9 g, 9.98 mmol) in 5 mL of CH₂Cl₂ over 1 min via cannula with stirring. The reaction mixture was allowed to stir an additional 17 h as the ice-water bath melted. Hünig's base (2.9 mL, 16.6 mmol) was added, and the reaction mixture was allowed to stir an additional 1.5 h at room temperature. The reaction mixture was then transferred to a separatory funnel containing 20 mL of water, and the organic layer was extracted with 60 mL of CH₂Cl₂. The organic layer was washed with 12 mL of ice-cold 13% w/v aqueous HCl, water (15 mL), and brine (20 mL). The residue upon drying and concentration of the organic layer was purified by flash chromatography on silica gel (0% to 25% to 50% EtOAc in hexanes) to afford tosylate 4.37 (1.014 g, 44%) as a light golden oil: R_{F} 0.43 (1:1 EtOAc/hexanes); ¹H NMR δ2.43 (s, 3H), 3.33 (s, 3H), 3.43-3.50 (m, 2H), 3.50-3.59 (m, 2H), 3.67 (t, 6.4 Hz, 2H), 4.15 (t, 6.4 Hz, 2H), 7.32 (d, 9.3 Hz, 2H), 7.78 (d, 9.3 Hz, 2H).

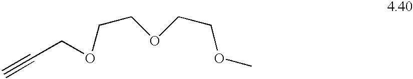

Compound 4.40: To an ice-water bath-cooled suspension of t-BuOK (95% w/w, 1.2 g, 10.2 mmol) in 7 mL of THF under argon was added a solution of diethylene glycol monomethyl ether (1.0 g, 8.32 mmol) in 1 mL of THF via cannula with stirring over 2 min. The homogeneous reaction mixture was allowed to stir an additional 5 min and was then added dropwise via cannula with vigorous stirring over 8 min to an ice-water bath-cooled solution of propargyl bromide (80% w/w, 1.85 mL, 16.6 mmol) in 27 mL of THF. The reaction mixture was allowed to stir overnight as the ice bath melted. The reaction mixture was transferred to a separatory funnel containing Et₂O (50 mL) and 60 mL of 45:15 brine/water. The layers were mixed, allowed to separate, and the aqueous layer was extracted with 2×50 mL of Et₂O. The combined organic layers were washed with 40 mL of brine and dried. The organic layer was concentrated to a residue that was purified by flash chromatography on silica gel (25% hexanes in Et₂O) to afford propargyl ether 4.40 (1.092 g, 84%) as a light golden liquid: R_{F} 0.53 (25% hexanes in Et₂O); ¹H NMR δ2.37 (t, 2.4 Hz, 1H), 3.30 (d, 0.8 Hz, 3H), 3.45-3.50 (m, 2H), 3.54-3.66 (m, 6H), 4.12 (dd, 2.4H, 0.8 Hz, 2H); ¹³C NMR δ58.19, 58.81, 68.91, 70.23, 70.36, 71.72, 74.37, 79.47; IR 3256, 2118, 1111 cm⁻¹; MS 159 (MH⁺), 127; HRMS m/e calc'd for C₈H₁₅O₃: 159.1021, found 159.1025.

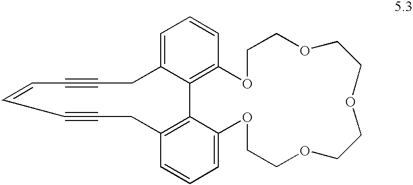

Compound 5.3: To a 5 mL round bottom flask containing compound 5.52 (5.9 mg, 14.0 μmol), a small stir bar and purified CuI (8.1 mg, 42.5 μmol) under argon was added 0.35 mL of dry, argon-sparged benzene, n-butylamine (12 μL, 0.121 mmol), and 1,2-dibromoethylene (91:9 cis/trans^{16a}, 1.25 μL, 13.7 ,μmol cis isomer). A solution of tetrakis(triphenylphosphine)palladium (0) (5.7 mg, 4.93 μmol) in 0.1 mL of dry, argon-sparged benzene under argon was added via cannula, and the dark yellow, homogeneous reaction mixture was allowed to stir at room temperature with the exclusion of light for 76 h. The resulting dark olive gum was resuspended in a small amount of MeOH and Et₂O and passed through a 0.5 g plug of silica gel. The plug was washed with Et₂O and the combined eluant was concentrated. The residue was purified by preparative TLC (1 mm silica gel plate, 6.5 cm [w]×20 cm [1 ], 5% hexanes in Et₂O) to afford enediyne 5.3 (0.5 mg, 9%) as a white film: ¹H NMR δ3.20-3.55 (m, 14H), 3.73 (dt, J=12.7, 3.0 Hz, 2H), 3.92 (dt, J=12.7, 3.0 Hz, 2H), 4.03-4.15 (m, 2H), 5.66 (s, 2H), 6.90 (d, J=9.0 Hz, 2H), 6.97 (d, J=7.8 Hz, 2H), 7.25 (t, J=8.6 Hz, 2H); IR 2197, 1670 cm⁻¹; MS 445 (MH⁺); HRMS m/e calc'd for C₂₈H₂₉O₅: 445.2015, found 445.2012.

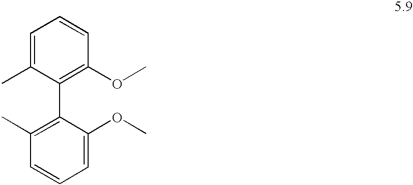

Compound 5.9:^{16b} A 200 mL receiving flask was charged with a pulverized mixture of iodide 5.10 (22.0 g, 88.7 mmol) and purified copper powder (21.4 g, 0.337 mol). An overhead mechanical stirrer with a water-cooled adapter sleeve was installed, and the reaction mixture was stirred at such a speed as to mix the components with minimal sloshing. The reaction mixture was then irradiated with a 60 W sun lamp at a distance of 9 inches and heated to 285° C. with a sand bath for 10 h. The reaction mixture was allowed to cool slightly, and the stirrer was removed. Upon cooling to room temperature, the reaction solids were broken into small pieces with a sturdy spatula, pulverized, triturated with 130 mL of Et₂O, and filtered through a coarse frit funnel. The solids were washed with 3×20 mL of Et₂O. Upon concentration of the combined ether extracts, the residue was purified by Kugelrohr distillation (118-125° C. ot, 0.17 torr) to afford biphenyl 5.9 (8.75 g, 82%) as a pale yellow solid: m.p. 122-123.5° C. (Lit. 122-123° C.^{16b}); R_{F} 0.63 (25% EtOAc in hexanes); ¹H NMR δ1.94 (s, 6H), 3.69 (s, 6H), 6.82 (d, J=8.9 Hz, 2H), 6.90 (d, J=8.9 Hz, 2H), 7.23 (t, J=8.9 Hz, 2H); ¹³C NMR δ19.55, 55.76, 108.31, 122.19, 126.20, 127.87, 138.19, 156.95; IR (KBr) 744 cm⁻¹; MS 243 (MH⁺); HRMS m/e calc'd for C₁₆H₁₉O₂: 243.1385, found 243.1373.

Purification of copper powder:^{∫}To a beaker containing 25 g of unpurified reddish-brown copper powder was added a solution of 3 g of I₂ in 150 mL of acetone and the resulting mixture was stirred manually for 7 minutes and the solids were then collected on a Buchner funnel. The solids were washed well with acetone and allowed to dry. The solids were then transferred to a beaker containing 150 mL of 1:1 conc. HCl/acetone, and the mixture was manually stirred for 8 minutes. The solids were again collected on a Buchner funnel, washed well with acetone, and allowed to dry. The solids were transferred to a small beaker and dried in vacuo with the exclusion of light for 8 h to afford 22.4 g of purified copper powder as a pinkish-red solid.

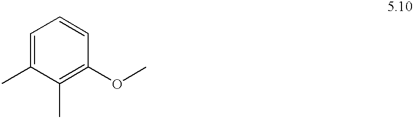

Compound 5.10:¹⁸ A 3-neck 1L round bottom flask equipped with an overhead mechanical stirrer, thermometer, and pressure-equalizing addition funnel was charged with 2-methoxy-6-methylaniline (22.5 g, 0.164 mol) and 276 mL of 1.8 M aqueous H₂SO₄. The solution was flushed with argon and then cooled with a salt/ice-water bath with vigorous stirring. The addition funnel was charged with a solution of pulverized, desiccated NaNO₂ (14.0 g, 0.198 mol) in 70 mL of water, and this solution was added over 22 min with stirring and cooling to <0° C. After an additional 30 min, the addition funnel was charged with a solution of KI (46.3 g, 0.278 mol) in 154 mL of water, and this was added with vigorous stirring over 20 min while maintaining the reaction mixture at <−5° C. After an additional 5 min, the cooling bath was removed, the addition funnel was removed, and the brown reaction mixture was gently heated to 70° C. over 50 min with vigorous stirring and then allowed to cool to room temperature. A small amount of solid NaHSO₃ (˜1 g) was added in portions with stirring, and the resulting yellowish-green supernatant and black settled oil was transferred to a separatory funnel. The oil was separated and the reaction mixture was extracted with 3×150 mL of Et₂O. The ether extracts and the oil were combined and washed with 2×75 mL 2% w/v NaOH and then dried over KOH pellets. The filtered residue upon concentration was purified via Kugelrohr distillation (80-100° C. ot, 0.5 torr) to yield iodide 5.10 (34.0 g, 84%) as a white solid that acquires a pinkish tint over time: m.p. 44.5-46.5° C. (Lit. 49° C.^{16b}); R_{F} 0.73 (25% EtOAc in hexanes); ¹H NMR δ2.46 (s, 3H), 3.87 (s, 3H), 6.63 (d, J=9.3 Hz, 1H), 6.87 (d, J=9.3 Hz, 1H), 7.16 (t, J=9.3 Hz, 1H); ¹³C NMR δ28.72, 56.44, 93.07, 107.97, 122.37, 128.67, 143.39, 158.11; IR (KBr) 611 cm⁻¹; MS 249 (MH⁺), 122; HRMS m/e calc'd for C₈H₉IO: 247.9698, found 247.9698.

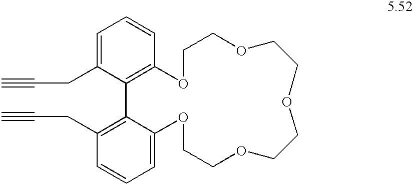

Compound 5.52: To a 5 mL round bottom flask containing bis(trimethylsilyl ether) 5.75 (4.7 mg, 8.33 μmol) and KF.2H₂O (9.6 mg, 0.102 mmol) under argon was added 0.2 mL of dry DMF. The reaction mixture was allowed to stir at room temperature for 6 h. The reaction mixture was then transferred to a separatory funnel, diluted with 10 mL of ice cold 3N HCl, and extracted with 3×15 mL of pentane. The combined pentane extracts were washed with 5 mL of cold 3N HCl, 5 mL of saturated aq. NaHCO₃, 5 mL of water, and 5 mL of brine. The pentane extracts were dried, filtered, and concentrated to afford compound 5.52 (2.5 mg, 68%) as a colorless oil. An analytical sample prepared via preparative TLC (1 mm silica gel plate, 10% hexanes in Et₂O) gave: ¹H NMR δ2.03 (t, J=3.0 Hz, 2H), 3.08 (dd, J=20.4, 3.0 Hz, 2H), 3.22 (dd, J=20.4, 3.0 Hz, 2H), 3.30-3.58 (m, 10H), 3.70 (dt, J=12.5, 3.3 Hz, 2H), 3.91 (dt, J=12.5, 3.3 Hz, 2H), 4.05-4.15 (m, 2H), 6.88 (d, J=7.9 Hz, 2H), 7.17-7.32 (m, 4H); ¹³C NMR δ22.58, 68.44, 70.02, 70.08, 71.25, 71.40, 82.22, 111.00, 120.59, 124.81, 128.58, 136.48, 156.00; IR 2123 cm⁻¹; MS 421 (MH⁺), 395; HRMS m/e calc'd for C₂₆H₂₉O₅: 421.2015, found 421.2010.

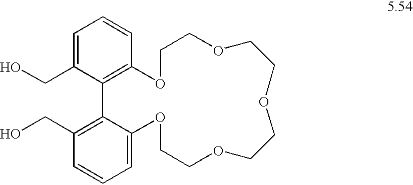

Compound 5.54: A 15 mL round bottom flask containing a small oval stir bar and tetrol 5.55 (0.174 g, 0.705 mmol) was equipped with a reflux condenser containing a Teflon sleeve fitted over the male joint. The apparatus was evacuated and flushed with argon twice. A freshly prepared aqueous NaOH solution (83 μL, 0.7 g/mL, 1.45 mmol) was added as a drop via gastight syringe to the reflux condenser and this was washed into the reaction vessel with 6 mL of dry THF. The reaction mixture was heated to reflux with stirring for 15 min, allowed to cool to room temperature, and a solution of tetraethylene glycol ditosylate (0.364 g, 0.724 mmol) in 1 mL of dry THF was added via cannula to the reaction mixture through the reflux condenser over 1 min. The reaction mixture was heated to reflux for 46 h with good stirring and then allowed to cool to room temperature. The pinkish, heterogeneous reaction mixture was then filtered through a plug of Celite on a medium frit, and the solids were washed with 4×10 mL of CHCl₃. The filtrate was transferred to a separatory funnel containing 50 mL of CHCl₃ and 50 mL of 40:5:5 brine/saturated aqueous NH₄Cl/water, and the layers were mixed and allowed to separate. The aqueous layer was extracted with 3×45 mL of CHCl₃, and the combined organic extracts were washed with 40 mL of 35:5 brine/water and 45 mL of brine. The residue upon concentration of the organic layer was purified by flash chromatography on silica gel (5% MeOH in Et₂O) to afford diol 5.54 (88.3 mg, 31%) as a pale yellow oil: R_{F} 0.3 (5% MeOH in Et₂O); ¹H NMR (MeOH-d₄) δ3.34 (s, 2H), 3.40-3.62 (m, 10H), 3.72 (dt, J=11.5, 4.2 Hz, 2H), 3.93 (dt, J=11.2, 4.2 Hz, 2H), 4.1-4.22 (m, 2H), 4.14 (d, J=13.0 Hz, 2H), 4.25 (d, J=13.0 Hz, 2H), 7.01 (d, J=8.3 Hz, 2H), 7.18 (d, J=8.3 Hz, 2H), 7.34 (t, J=8.3 Hz, 2H); ¹³C NMR (MeOH-d₄) δ62.94, 69.37, 70.82, 72.01 (2C), 112.65, 120.89, 125.27, 129.56, 142.43, 157.27; IR 1137 cm⁻¹; MS 404 (M⁺), 387; HRMS m/e calc'd for C₂₂H₂₈O₇: 404.1835, found 404.1827.

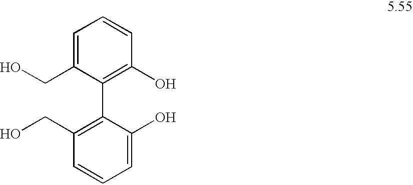

Compound 5.55:¹⁹ A 25 mL round bottom flask containing a small oval stir bar, dilactone 5.68 (51.8 mg, 0.218 mmol) and lithium aluminum hydride (68.5 mg, 1.81 mmol) was fitted with a reflux condenser containing a Teflon sleeve around the male joint and the entire apparatus was evacuated and argon flushed twice. 12 mL of dry THF was added through the reflux condenser and the reaction mixture was heated to reflux with stirring for 12 h. The heterogeneous reaction mixture was cooled to 0° C. and then carefully treated with 1.5 mL of saturated aqueous disodium tartrate followed by 1.5 mL of saturated aqueous NH₄Cl, and then allowed to stir vigorously for 15 min. The reaction mixture was allowed to settle and then was filtered through a plug of Celite on a glass frit. The solids in the reaction vessel were washed with 2×10 mL EtOAc, filtered and the filtered solids were washed with 5 mL EtOAc. The combined filtrates were transferred to a separatory funnel and diluted with 35 mL of EtOAc and 35 mL of 33:2 brine/water. The layers were mixed and allowed to separate. The aqueous layer was saturated with solid NH₄Cl and extracted with 5×20 mL of EtOAc. The combined EtOAc extracts were washed with 20 mL of brine, dried, filtered and the residue upon concentration was purified by flash chromatography on silica gel (2% MeOH in Et₂O) to afford tetrol 5.55 (38.2 mg, 71%) as a white solid: m.p. 159-162° C.; R_{F} 0.47 (2% MeOH in Et₂O); ¹H NMR (DMSO-d₆) δ3.95 (d, J=14.4 Hz, 2H), 4.13 (d, J=14.4 Hz, 2H), 4.82 (s(br), 2H), 6.75 (d, J=7.6 Hz, 2H), 6.97 (d, J=7.6 Hz, 2H), 7.12 (t, J=7.6 Hz, 2H), 8.90 (s, 2H); ¹³C NMR (DMSO-d₆) δ60.81, 113.38, 116.86, 120.95, 127.48, 142.03, 154.05; IR (KBr) 3333 (br) cm⁻¹; MS 247 (MH⁺), 229; HRMS m/e calc'd for C₁₄H₁₄O₄: 246.0892, found 246.0892.

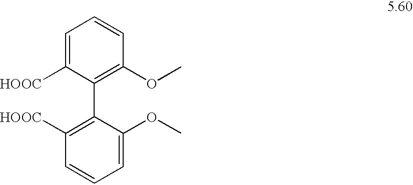

Compound 5.60:²⁰ A 3-neck 250 mL round bottom flask equipped with a pressure-equalizing addition funnel and an oval stir bar was charged with compound 5.9 (202 mg, 0.835 mmol) and 50 mL of water. The entire apparatus was evacuated and filled with argon. The suspension was then heated near reflux with moderate stirring to avoid sloshing. The addition funnel was charged with 60 mL of freshly prepared 2% w/v aqueous KMnO₄ (7.59 mmol), and this was added in 10 mL aliquots over 50 min with continued heating and stirring. The reaction mixture was allowed to stir an additional 2 h 40 min and was then cooled to 0° C. with stirring. A freshly prepared saturated aqueous solution of NaHSO₃ (15 mL) was then added with stirring. After being stirred an additional 2 h, the reaction mixture had warmed to room temperature. The colorless, inhomogeneous reaction mixture was acidified to ˜pH 2 (litmus paper) with concentrated HCl to yield a colorless, homogenous solution. The reaction mixture was saturated with solid NaCl, and the supernatant was transferred to a separatory funnel and extracted with 4×50 mL of Et₂O. The combined ether extracts were carefully extracted with 3×50 mL of saturated aq. NaHCO₃. The ether extracts were washed with 50 mL of 45:5 brine/water and brine (50 mL), dried, and concentrated to yield 85.2 mg of recovered 5.9. The combined alkaline extracts were acidified to pH 2 with concentrated HCl (pH meter), carefully saturated with NaCl, and the supernatant was extracted with 3×50 mL of Et₂O. These combined ether extracts were washed with brine (50 mL), dried, filtered, and concentrated to afford diacid 5.60 (49.8 mg, 34% based on recovered starting material) as a white solid: m.p. >150° C. (dec., Lit. 295-298° C. dec.²⁰); R_{F} 0-0.25 (5% MeOH in Et₂O); ¹H NMR (MeOH-d₄) δ3.64 (s, 6H), 7.14 (dd, J=8.7, 0.5 Hz, 2H), 7.33 (t, J=8.7 Hz, 2H), 7.54 (dd, J=8.7, 0.5 Hz, 2H); ¹³C NMR (MeOH-d₄) δ56.36, 115.35, 123.01, 128.84, 129.29, 133.21, 158.45, 170.76; IR (KBr) 1698 (br) cm⁻¹; MS 303 (MH⁺), 285; HRMS m/e calc'd for C₁₆H₁₅O₆: 303.0869, found 303.0859.

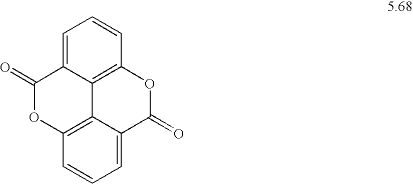

Compound 5.68:¹⁹ To a 200 mL receiving flask containing an oval stir bar and diacid 5.60 (645 mg, 2.14 mmol) was added 35 mL of concentrated HBr and 35 mL of glacial acetic acid. The headspace above the reaction mixture was blanketed well with argon and an argon-flushed reflux condenser was attached. The reaction mixture was heated to reflux with good stirring for 2 h at which time the reaction had become quite cloudy. The heterogeneous reaction mixture was then cooled to 0° C. with stirring and filtered through a medium frit funnel. The collected solids were washed with 2×10 mL water and 2×10 mL cold Et₂O, and dried in vacuo over P₂O₅ for 30 min to afford dilactone 5.68 (326 mg, 64%) as a white solid that is insoluble in many room temperature solvents: m.p. >255° C. (Lit. 365° C.¹⁹); IR (KBr) 1747 cm⁻¹; MS 239 (MH⁺); HRMS m/e calc'd for C₁₄H₇O₄: 239.0344, found 239.0344.

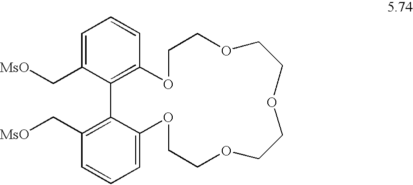

Compound 5.74: A 5 mL round bottom flask containing a small stir bar and mesyl chloride (43 μL, 0.556 mmol) in 0.4 mL of dry CH₂Cl₂ under argon was cooled to 0° C. A solution of diol 5.54 (49.2 mg, 0.122 mmol) and triethylamine (85 μL, 0.611 mmol) in 0.4 mL of dry CH₂Cl₂ under argon was added with stirring in seven portions over 13 min using a short 19-gauge cannula. To the flask that held diol 5.54 and triethylamine was added an additional 0.15 mL of dry CH₂Cl₂ and this rinse was transferred via cannula to the reaction mixture in two portions over 4 min. The reaction mixture was allowed to stir at 0° C. for an additional hour and then at room temperature for 3 h 20 min. The reaction mixture was added to a separatory funnel containing 40 mL of EtOAc and 30 mL of 20:10 saturated aq. NH₄Cl/ice-water, and the layers were mixed and allowed to separate. The aqueous layer was extracted with 3×30 mL of EtOAc and the combined organic extracts were washed with 20 mL of brine. The residue upon drying and concentration of the organic layer was purified by preparative TLC (1 mm silica gel plate, 20×20 cm, 5% MeOH in Et₂O) to afford dimesylate 5.74 (41.5 mg, 61%) as a colorless oil: R_{F} 0.62 (10% MeOH in Et₂O); ¹H NMR δ2.68 (s, 6H), 3.32-3.58 (m, 10H), 3.71 (dt, J=12.2, 4.3 Hz, 2H), 3.96 (dt, J=12.2, 4.3 Hz, 2H), 4.10-4.23 (m, 2H), 4.77 (d, J=12.2 Hz, 2H), 4.91 (d, J=12.2 Hz, 2H), 7.05 (d, J=8.5 Hz, 2H), 7.14 (d, J=8.5 Hz, 2H), 7.36 (t, J=8.5 Hz, 2H); ¹³C NMR δ36.90, 68.47, 69.79, 69.85, 71.10, 71.24, 113.37, 121.55, 124.52, 129.33, 133.80, 156.20; IR 1359, 1179 cm⁻¹; MS 560 (M⁺), 497, 465, 369; HRMS m/e calc'd for C₂₄H₃₂O₁₁S₂: 560.1386, found 560.1386.

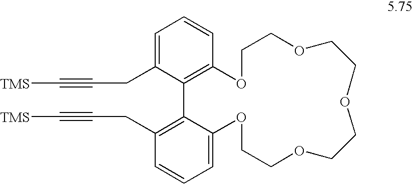

Compound 5.75: A 25 mL 3-neck round bottom flask containing a small stir bar and equipped with a reflux condenser fitted with a Teflon sleeve over the male joint was evacuated and flushed with argon three times and then charged with 0.3 mL of dry THF and trimethylsilylacetylene (160 μL, 1.13 mmol). Isobutylmagnesium bromide (2.0 M, 425 μL, 0.85 mmol) was added over 2 min and the reaction mixture was allowed to stir at room temperature for 1 h. An additional 0.2 mL of dry THF was added and the thick suspension was then briefly (5 min) heated to reflux and allowed to cool. A stirring suspension of pulverized CuBr.Me₂S (47.9 mg, 0.233 mmol) in 0.7 mL of dry THF under argon was added in one portion via an 18-gauge cannula. Stirring was continued an additional 10 min and again the reaction mixture was briefly refluxed and then allowed to cool to room temperature. A solution of dimesylate 5.74 (15.9 mg, 28.4 μmol) in 0.3 mL of dry THF was added via cannula, and the reaction mixture was heated to reflux for 13.5 h. Upon cooling to room temperature, the brownish-yellow, heterogeneous reaction mixture was resuspended in 1.0 mL of THF. The reaction mixture was quenched by the addition of 2.0 mL of pH 8.3 saturated aqueous NH₄Cl, and the quenched reaction was stirred for 30 min and then transferred to a separatory funnel containing 35 mL of Et₂O and 25 mL of 10:10:5 pH 8.3 saturated aqueous NH₄Cl/brine/water. The layers were mixed, allowed to separate, and the aqueous layer was extracted with 3×20 mL of Et₂O. The combined ether extracts were washed with 10 mL of pH 8.3 saturated aq. NH₄Cl, 2×10 mL of water and 15 mL of brine. The washed ether extracts were then passed through a 2 g plug of silica, and the plug was washed with 15 mL of Et₂O. The combined filtrates were dried in vacuo, filtered, and concentrated. The residue was purified by preparative TLC (1 mm silica gel plate, 20 cm [1]×10 cm [w], 20% hexanes in Et₂O) to afford bis(trimethylsilyl ether) 5.75 (5.1 mg, 32%) as a colorless oil: R_{F} 0.4 (20% hexanes in Et₂O); ¹H NMR δ0.12 (s, 18H), 3.17 (d, J=19.6 Hz, 2H), 3.27 (d, J=19.6 Hz, 2H), 3.34-3.60 (m, 10H), 3.62 (dt, J=11.1, 3.7 Hz, 2H), 3.93 (dt, J=11.1, 3.7 Hz, 2H), 4.05-4.17 (m, 2H), 6.87 (dd, J=9.3, 0.9 Hz, 2H), 7.16-7.32 (m, 4H); ¹³C NMR δ0.11, 24.09, 68.39, 70.00, 71.19, 71.37, 86.52, 104.61, 110.91, 120.61, 124.86, 128.42, 136.63, 155.82; IR 2183, 1260, 847 cm⁻¹; MS 565 (MH⁺), 549, 467; HRMS m/e calc'd for C₃₂H₄₅O₅Si₂: 5.65.2806, found 565.2801.

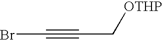

2-(3-bromo-2-propynyloxy)tetrahydro-2H-pyran:²¹ To a solution of THP-protected propargyl alcohol (2.0 mL, 13.9 mmol) in 90 mL of acetone were added sequentially, in one portion NBS (2.9 g, 16.26 mmol) and AgNO₃ (0.248 g, 1.53 mmol) with stirring. The reaction mixture was allowed to stir at room temperature for 1 h. The heterogeneous reaction mixture was then shaken with 40 mL of water and extracted with 4×50 mL of EtOAc. The combined organic extracts were washed with water (50 mL) and brine (50 mL). The residue upon drying and concentration of the organic layer was purified by Kugelrohr distillation (6 mm, 90-92 ot) to afford the title compound (2.41 g, 80%) as a colorless oil: ¹H NMR δ1.43-1.87 (m, 6H), 3.45-3.55 (m, 1H), 3.63-3.86 (m, 1H), 4.25 (dd, J=17.1, 4.1 Hz, 2H), 4.77 (t, 4.1 Hz, 1H); ¹³C NMR δ18.91, 25.26, 30.10, 45.50, 54.86, 61.92, 76.13, 96.80; MS 219 (MH⁺), 133, 117.

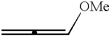

Methoxyallene:²² A 15 mL round bottom flask was charged with propargyl methyl ether (5.0 mL, 65.4 mmol) and t-BuOK (95% w/w, 0.75 g, 6.34 mmol). The vessel was flushed well with argon and an argon-flushed reflux condenser fitted with a Teflon sleeve was attached. The reaction mixture was heated to reflux with efficient stirring for 4 h. Upon cooling to near room temperature, the reflux condenser was replaced with a short path distillation head and the orange-brown reaction mixture was distilled to dryness to afford the title compound as a colorless distillate (3.75 g, 90%) with spectral properties identical to those reported by Weiberth and Hall.²²

Alkali Metal Picrates: Sodium and potassium picrate were prepared according to a reported method.²³ Lithium picrate was prepared by a modification of this method using lithium hydroxide. The salts were vacuum-dried (0.3 mm) at 175° C. for 2 days. ¹H NMR analysis (d₆-DMSO) determined that lithium and potassium picrate were anhydrous while sodium picrate was a monohydrate.

Determination of Alkali Metal Ion Complex Association Constants: The general procedures employed were after those developed by Cram.²⁴ Distilled, demineralized water and spectrophotometric grade CHCl₃ and MeCN were used. CHCl₃ and water were saturated with each other prior to solution preparations as a means of preventing volume changes of the phases during the extractions. All glassware was washed with nonionic detergent, rinsed well with tap water, demineralized water and methanol followed by drying in an oven (115° C.) or a vacuum dessicator over P₂O₅. All operations were conducted at 23-24° C. In a typical extraction, 250 μL of a 3.0 mM aqueous metal picrate solution and 250 μL of host solution (various concentrations from 3 to 30 mM; see text for specific values) in CHCl₃ were placed in a 0.5 dram vial which was immediately stoppered with a screw-cap. The vials were centrifuged for 1 min to drive the CHCl₃ layer to the bottom of the vial. The vials were then vortexed for 1 min with a Baxter S/P vortex mixer followed by centrifugation at high speed for 15 min with an International Clinical Centrifuge to effect complete phase separation. A 50 μL aliquot (gastight syringe, volume measured by difference) of the CHCl₃ layer was removed from the middle and bottom of the vial and dispensed into a 1.0 mL volumetric tube and the volume was brought up to the mark with MeCN (dilution factor=20). To ensure no contamination by the aqueous layer, the syringe needle was washed with a stream of demineralized water and dried with a Kimwipe prior to dispensing the aliquot. The diluted CHCl₃ aliquot was homogenized by several inversions and transferred to a quartz cuvette dedicated for either lithium, sodium or potassium extractions. Absorbance was measured at 380 nm against a blank prepared in a manner analogous to the extraction procedure above using demineralized water in place of aqueous metal picrate and host-free CHCl₃. The extractability of metal picrates by CHCl₃ in the absence of host was determined as above with CHCl₃ that was free of host. The absorbance of the CHCl₃ layer (after dilution and against the aforementioned blank) for each metal picrate extracted was found to be 0.002 and this value was subtracted from all absorbance readings obtained from host-containing extraction experiments. An aliquot of the host solution in CHCl₃ was similarly diluted with MeCN and its absorbance was measured. This value was also subtracted from the absorbance value obtained from the extraction experiments. The concentration of metal picrate in the CHCl₃ layer at equilibrium was determined using the absorbance of the diluted CHCl₃ layer (corrected for absorbance due to the host and CHCl₃-mediated alkali metal picrate extraction) and the Beer's law equation (A=εbc; b=1.0 cm) with each metal picrate exhibiting an extinction coefficient (ε) of 16,900 M⁻¹ cm⁻¹ at 380 nm in MeCN as determined previously by Cram and co-workers.²⁵ Complex association constants (Ka's) were determined by the method of Cram²⁵ from extraction constants (Ke's; derived from absorbance measurements made on the CHCl₃ layer) and distribution constants (Kd's; previously determined by Cram and co-workers²⁵) as described in the text (see Section 2.5.2).

Preparation of Alkali Metal Ion DNA Solutions (M.DNAs): A strain of DH5αE. Coli which contains the pGAD424/p28 plasmid was incubated in Luria-Bertani media containing 400 μg/mL sodium ampicillin. Cells were collected, lysed, and the plasmid DNA was isolated with a QIAGEN miniprep spin kit according to the manufacturer's instructions. This afforded the DNA as an aqueous solution in demineralized, sterile water. 10 μL aliquots of this solution was gently mixed with 212.2 μL of a pH 7.4 sterile, aqueous alkali metal phosphate solution. The plasmid DNA so obtained was determined spectrophotometrically to be 17 μM base pair and typically contained 75-85% supercoiled (Form I) DNA as determined by agarose gel electrophoresis. For EC₂₅ experiments, the concentration of alkali metal ion before addition of the aqueous DNA solution was 20 mM. For all other experiments, the concentration of lithium or sodium ions prior to addition of the aqueous DNA solution was 0, 1.5, 3.0 or 6.0 mM in 18-20 mM TRIS phosphate. It is worth noting that DNA solutions that were prepared using aqueous alkali metal acetates exhibited significantly less cleavage upon incubation with propargylic sulfone-containing agents than solutions prepared as above which employed aqueous alkali metal phosphates.

DNA Cleavage Protocol: In a typical cleavage experiment, 2 μL of a freshly prepared solution of the agent in d₆-DMSO (or, for control reactions, d₆-DMSO that was free of agent) was added to a small, sterile eppindorf tube followed by 14 μL of an aqueous solution of alkali metal ion-containing DNA prepared as above. The contents were mixed by brief (5 seconds) centrifugation at 6,000 rpm and allowed to stand at room temperature for 19 h. Tubes were then heated for 90 s at 70° C. Upon cooling, 2 μL of 8×loading dye (0.13 g/mL each of bromophenol blue and xylene cyanole in 30% glycerol-water) was added to each tube, the contents were gently homogenized and briefly centrifuged, and 5 μL of the resulting solution was loaded onto a 0.7% w/v agarose gel. The DNA cleavage products were separated by electrophoresis in 1×TBE running buffer at 45 volts for 2.25 h. The agarose gel was stained for 15 min in TBE buffer that contained 0.25 μg/mL ethidium bromide and then destained in distilled water for 15-30 min. The gel was scanned with a Molecular Dynamics Fluorimager and the quantities of Forms I, II and III DNA were assessed with the ImageQuaNT software program. The degree of cleavage of Form I DNA was determined as described in the text (see Section 2.6.2).

Isomerization Facility Experiments: To a small vial containing a small stir bar and a solution (58-82 μL) of propargylic sulfone 3.1, 3.2 or 3.3 in MeOH (0.1 M) was added a solution (58-82 μL) of aqueous pH 7.4, 0.1 M potassium phosphate solution such that the final sulfone concentration was 50 mM. A screw-cap was installed on the reaction vial and the reaction mixture was stirred at room temperature for 4.5 h. The reaction was diluted with 0.5 mL of water and extracted with CHCl₃ (3×1 mL, by pipet). The organic layers were concentrated, and the residue was resuspended in CDCl₃ and analyzed by ¹H NMR. The isomerized allenylic sulfone derived from compound 3.2 (para-substituted species) was identified by a distinctive doublet (7.47 ppm, J=8.8 Hz). The isomerized allenylic sulfones derived from the meta-substituted compounds (3.1 and 3.3) were also identified by distinctive doublets (7.65 ppm, J=7.9 Hz for sulfone 3.1; 7.62 ppm, 8.2 Hz for sulfone 3.3). Integration of the aforementioned peaks and the corresponding peaks due the propargylic sulfone starting material lead to the values for extent of isomerization for each compound which are shown in Table 3.5.

Cycloaromatization Experiments: In a typical experiment, a stock solution was prepared by addition of the following to a large vial: enediyne 4.7 or 4.9 (490 μL, 50 mM solution in d₆-DMSO, 24.5 μmol); 245 μL of either D₂O, 7.5 M LiCl in D₂O, or a saturated solution of NaCl or KCl in D₂O; caffeine (210 μL, 20.0 mg/mL in d₆-DMSO, 21.6 μmol); 1,4-cyclohexadiene (455 μL, 4.81 mmol); and d₆-DMSO (3,500 μL). The mixture was thoroughly homogenized by vortexing and 700 μL aliquots of the resulting solution were placed into three well-cleaned (nonionic detergent) vacuum hydrolysis tubes. A fourth 700 μL aliquot was placed into an NMR tube (to serve as the zero hour time point). The final enediyne concentration in the stock reaction mixture was 5 mM. The vacuum hydrolysis tube caps were installed with unused Viton O-rings and screwed onto the tubes. Each tube was freeze-pump-thawed three times under argon to remove traces of oxygen. The tubes were then immersed simultaneously into an oil bath kept at 152° C. After a period of time, a tube was withdrawn from the oil bath, allowed to cool to room temperature, and roughly one half of the contents were transferred by pipet to an NMR tube. The cap was replaced on the tube, oxygen was removed as before, and the tube was immersed in the oil bath anew. In this manner, seven time points during the course of the cycloaromatization reaction were generated (i.e., 0, 3 ,6 ,9, 12, 15, and 20 hours). The remainder of the stock solution in the vial was processed as above and served as a duplicate run, using the original time=0 hours data point for both runs. The withdrawn aliquots were analyzed via ¹H NMR (250 MHz, referenced to DMSO) using the following acquisition parameters: pulse width=8.5 μsec; receiver delay=2 sec; receiver gain=8; and number of scans=90. The spectrum was printed such that the internal standard peak (caffeine methyl group that is furthest downfield) was the largest peak and the printed area covered the range from 3.5 to 4.9 ppm. For each time point, the peak areas for the internal standard (s, 3.86 ppm), propargylic methylene of the starting enediyne (for crown 4.7: s, 4.35 ppm; for podand 4.9: s, 4.34 ppm), and benzylic methylene of the H atom-quenched cyclization product (for crown 4.8: s, 4.56 ppm; for podand 4.41: s, 4.53 ppm) were cut form the spectrum and weighed on an analytical balance. The peak area ratio (PAR) was defined as either enediyne to internal standard or o-xylyl compound to internal standard. The PARs so obtained were used to calculate the rate constants for disappearance of enediyne starting materials or the rate constants for the initial appearance of o-xylyl cyclization products as described in Section 4.5.2. The identity of the H atom-quenched cyclization products were confirmed with solvent-removed residues by comparison to literature ¹H NMR values²⁶ and by MS: HRMS m/e for compound 4.8 (C₁₈H₂₉O₆) calc'd 341.1964, found 341.1966; LRMS for compound 4.41 gave m/e 343 (MH⁺, 20% RA).

References

The following references, to the extent that they provide exemplary procedural or other information or details supplementary to those set forth herein, are specifically incorporated herein by reference

1.1. Sporn, M. Cancer Res. 1991, 51, 6215.

1.2. Calabresi, P.; Parks, R. E. Jr. In Goodman and Gilman's The Pharmacological Basis of Therapeutics, Seventh Ed., Gilman, A. G.; Goodman, L. S.; Rall, T. W.; Murad, F. Eds., MacMillan, New York, 1985, 1241.

1.3. Larsen, I. K., In A Textbook of Drug Design and Development, Krogsgaard-Larsen, P.; Bundgaard, H. Eds., Harwood, Philadelphia, 1991, 193.

1.4. Denny, W. A. Curr. Pharm. Design 1996, 2, 281.

1.5. Patel, N. H.; Rothenberg, M. L. Invest. New Drugs 1994, 12, 1.

1.6. enediynes: (a) Edo. K.; Mizugaki, M.; Koide, Y.; Seto, H.; Purihata, H.; Otake, N.; Ishida, N. Tetrahedron Lett. 1985, 26, 331. (b) Lee, M. D.; Dunne, T. S.; Siegel, M. M.; Chang, C. C.; Morton, G. O.; Borders, D. B. J. Am. Chem. Soc. 1987, 109, 3464. (c) Golik, J.; Clardy, J.; Dubay, G.; Groenwald, G.; Kawaguchi, H.; Konishi, M.; Krishnan, B.; Ohkuma, H.; Saitoh, K.; Doyle, T. W. J. Am. Chem. Soc. 1987, 109, 3461. CC-1065/duocarmycins: (a) Hanka, L. J.; Dietz, A.; Gerpheide, S. A.; Kuentzel, S. L.; Martin, D. G. J. Antibiot. 1978, 31, 1211. (b) Ichimura, M.; Muroi, K.; Asano, K.; Kawamoto, I.; Tomita, F.; Morimoto, M.; Nakano, H. J. Antibiot. 1988, 41, 1285. (c) Ichimura, M.; Ogawa, T.; Katsumata, S.; Takahashi, K.; Takahashi, I.; Nakano, H. J. Antibiot. 1991, 44, 1045.

1.7. Hung, D. T.; Jamison, T. F.; Schreiber, S. L. Chemistry & Biology 1996, 3, 623.

1.8. for a review of ADEPT, see: Niculescu-Duvaz, I.; Springer, C. J. Current Medicinal Chemistry 1995, 2, 687.

1.9. Woodburn, K. W.; Fan, Q.; Kessel, D.; Wright, M.; Mody, T. D.; Hemmi, G.; Magda, D.; Sessler, J. L.; Dow, W. C.; Miller, R. A.; Young, S. A. J. Clin. Laser Medicine & Surgery 1996, 14, 343.

1.10. Woodburn, K. W.; Fan, Q.; Kessel, D.; Luo, Y.; Young, S. W. J. Invest. Derm. 1998, 110, 746.

1.11. Denny, W. A. Curr. Pharm. Design 1996, 2, 281.

1.12. Tietze, L. F.; Newman, M. Mollers, T. Fischer, R.; Glusenkamp, K. -H.; Rajewsky, M.; Jahde, E. Cancer Res. 1989, 49, 4179.

1.13. Palmer, B. D.; Wilson, W. R.; Atwell, G. J.; Schultz, D.; Xu, X. Z.; Denny, W. A. J. Med. Chem. 1994, 37, 2175.

1.14. Knox, R. J.; Friedlos, F.; Boland, M. P. Cancer Metastasis Rev. 1993, 12, 195.

1.15. for a review of the cell cylce, see: Murray, A.; Hunt, T. The Cell Cycle W. H. Freeman, New York, 1993.

1.16. Larsen, I. K., In A Textbook of Drug Design and Development, Krogsgaard-Larsen, P.; Bundgaard, H. Eds., Harwood, Philadelphia, 1991, p. 232.

1.17. for a review of mitomycins, see: Verweij, J. Cancer Chemotherapy and Biological Response Modifiers 1997, 17, 46.

1.18. Ali-Osman, F.; Antoun, G.; Wang, H.; Ragagopal, S.; Gagucas, E. Mol. Pharm. 1996, 49, 1012.

1.19. Mello, J. A.; Acharya, S.; Fishel, R.; Essigman, J. M. Chemistry & Biology 1996, 3, 579.

1.20. for a review of sequence-specific DNA minor groove binders, see: Bailly, C.; Chaires, J. B. Bioconjugate Chem. 1998, 9, 513.

1.21. White, S.; Scewczyk, J. W.; Turner, J. M.; Baird, E. E.; Dervan, P. B. Nature, 1998, 391, 468.

1.22. Trauger, J. W.; Baird, E. E.; Dervan, P. B. Nature, 1996, 382, 559.

1.23. Blackburn, E. Nature 1991, 350, 569.

1.24. Wheelhouse, R. T.; Sun, D.; Han, H.; Han, F. X.; Hurley, L. H. J. Am. Chem. Soc. 1998, 120, 3261.

1.25. Long, B. H.; Golik, J.; Forenza, S.; Ward, B.; Rehfuss, R.; Dabrowiak, J. C.; Catino, J. J.; Musial, S. T.; Brookshire, K. W.; Doyle, T. W. Proc. Natl. Acad. Sci. USA 1989, 86, 2.

1.26. (a) enediynes, see: reference 6a. (b) bleomycin, see: Stubbe, J.; Kozarich, J. W. Chem. Rev. 1987, 87, 1107. (c) leinamycin, see: Mitra, K.; Kim. W.; Daniels, S.; Gates, K. S. J. Am. Chem. Soc. 1997, 119, 11691.

1.27. Shields, T. P.; Barton, J. K. Biochemistry 1995, 34, 15037.

1.28. Singh, U. S.; Scannell, R. T.; An, H.; Carter, B. J.; Hecht, S. M. J. Am. Chem. Soc. 1995, 117, 12691.

1.29. for a review of radical-induced oxidative damage, see: Pogozelzki, W. K.; Tullius, T. D. Chem. Rev. 1998, 98, 1089.

1.30. for an example, see: Schulz, W. G.; Islam,. I.; Skibo, E. B. J. Med. Chem. 1995, 38, 109.

1.31. for a review of DNA modification due to nucleobase alkylation, see: Burrows, C. J.; Muller, T. D. Chem. Rev. 1998, 98, 1109.

1.32. for a review, see: Sinha, B. K. Drugs 1995, 49, 11.

1.33. (a) calicheamicin γ₁^{I}: Zhao, B.; Konno, S.; Wu, J. M.; Oronsky, A. L. Cancer Lett. 1990, 50, 141. (b) neocarzinostatin: Maeda, H. Anticancer Res. 1981, 1, 175. (c) dynemicin A: Konishi, M.; Ohkuma, H.; Tsuno, T.; Oki, T.; VanDuyne, G. D.; Clardy, J. J. Am. Chem. Soc. 1990, 112, 3715.

1.34. for calicheamicin γ₁^{I}, see: (a) Walker, S.; Landovitz, R.; Ding, W. -D.; Ellestad, G. A.; Kahne, D. Proc. Natl. Acad. Sci. USA 1992, 89, 4608. (b) Hangeland, J. J.; De Voss, J. J.; Heath, J. A.; Townsend, C. A.; Ding, W. -D.; Ashcroft, J. S.; Ellestad, G. A. J. Am. Chem. Soc. 1992, 114, 9200.

1.35. Grissom, J. W.; Gunawardena, G. U.; Klingberg, D.; Huang, D. Tetrahedron, 1996, 52, 6453.

1.36. Bergman, R. G. Acc. Chem. Res. 1973, 6, 25.

1.37. Dedon, P. C.; Goldberg, I. H. Chem. Res. Tox. 1992, 5, 311.

1.40. Nicolaou, K. C.; Skokotas, G.; Maligres, P.; Zucarello, G.; Schweiger, E. J.; Toshima, K.; Wendeborn, S. Angew. Chem., Int. Ed. Engl. 1989, 28, 1272.

1.41. Gupta, R.; Xie, G.; Lown, J. W. Gene 1994, 149, 81.

1.44. Dai, W -M.; Fong, K. C. Tetrahedron Lett. 1995, 36, 5613.

1.52. Bishayee, A.; Kulkarni, S. P.; Roy, K.; Chatterjee, M. Cancer Investigation 1998, 16, 291.

1.67. Chen, L. B. Annu. Rev. Cell. Biol. 1988, 4, 155.

1.68. Koya, K.; Li, Y.; Wang. H.; Ukai, T.; Tatsuta, N.; Kawakami, M.; Shishido, T.; Chen, L. B. Cancer Res. 1996, 56, 538.

1.69. Sharma, V.; Crankshaw, C. L.; Piwnica-Worms, D. J. Med. Chem. 1996, 39, 3483.

2.1. Nicolaou, K. C.; Skokotas, G.; Maligres, P.; Zucarello, G.; Schweiger, E. J.; Toshima, K.; Wendeborn, S. Angew. Chem., Int. Ed. Engl. 1989, 28, 1272.

2.2. Braverman, S.; Duar, Y.; Segev, D. Tetrahedron Lett. 1976, 3181.

2.3. Maxam, A. M.; Gilbert, W. Methods Enzymol. 1980, 65, 499.

2.5. Kerwin, S. M. Tetrahedron Lett. 1994, 35, 1023.

2.9. Czech, B.; Quici, S.; Regen, S. L. Synthesis, 1980, 113.

2.12. Martell, A. E.; Hancock, R. D.; Eds. Metal Complexes in Aqueous Solutions, Plenum Press, New York, 1996, 226.

2.13. Helgeson, R. C.; Weisman, G. R.; Toner, J. L.; Tamowski, T. L.; Chao, Y.; Mayer, J. M.; Cram, D. J. J. Am. Chem. Soc. 1979, 101, 4928.

2.14. Ghasemi, J.; Shamsipur, M. J. Coord. Chem. 1993, 28, 231.

2.16. Newcomb, M.; Timiko, J. M.; Walba, D. M.; Cram, D. J. J. Am. Chem. Soc. 1977, 99, 6392.

3.4. Tsukube, H. Coordination Chem. Rev. 1996, 148, 1.

4.26. Melikyan, G. G.; Khan, M. A.; Nicholas, K. M. Organometallics 1995, 14, 2170.

4.43. Wrasidlo, W.; Hiatt, A.; Mauch, S.; Merlock, R. A.; Nicolaou, K. C. Acta Oncologica 1995, 34, 157.

5.14. Sugii, Y.; Shindo, H. J. Pharm. Soc. Japan 1934, 54, 829.

7.1. Linstrumelle, G.; Krieger, J. K.; Whitesides, G. M. Org. Synth. 1976, 55, 103.

7.2. Still, W. C.; Kahn, M.; Mitra, A. J. Org. Chem. 1978, 43, 2923.

7.3. Nicolaou, K. C.; Skokotas, G.; Maligres, P.; Zucarello, G.; Schweiger, E. J.; Toshima, K.; Wendeborn, S. Angew. Chem., Int. Ed. Engl. 1989,28, 1272.

7.4. Basak, A.; Khamrai, U. K. Tetrahedron Lett. 1995, 36, 7913.

7.5. Kerwin, S. M. Tetrahedron Lett. 1994, 35, 1023.

7.6. Crombie, L.; Haigh, D.; Jones, R. C. F.; Mat-Zin, Ab. R. J. Chem Soc. Perkins Trans. 1 1993, 17, 2047.

7.7. (a) Fraser, M. M.; Raphael, R. A. J. Chem. Soc. 1955, 4280. (b) see Reference 5.

7.8. McPhee, M. M.; Kerwin, S. M. J. Org Chem. 1996, 61, 9385.

7.9. Eichinger, W. V.; Musso, H.; Eichhorn, K. D.; Mattern, G. J. Prakt. Chem./Chem.-Ztg. 1998, 340, 140.

7.10. Scobie, M.; Threadgill, M. D. J. Org. Chem. 1994, 59, 7008.

7.11. Simon, D. Z.; Brookman, S.; Beliveau, J.; Salvador, R. L. J. Pharm. Sci. 1977, 66, 431.

7.12. Morgan, G. T.; Porter, C. R. J. Chem. Soc. 1926, 1258.

7.13. Bailey, W. J.; Fujiwara, E. J. Am. Chem. Soc. 1955, 77, 165.

7.14. Gleiter, R.; Rittinger, S.; Langer, H. Chem Ber. 1991, 124, 357.

7.15. Chao, Y.; Weisman, G. R.; Sogah, G. D. Y.; Cram, D. J. J. Am. Chem. Soc. 1979, 101, 4948.

7.16 (a) for separation of the commercially available 1:1 mixture of cis and trans-1,2-dichloroethylene, see: Miller, S. I.; Noyes, R. M. J. Am. Chem. Soc. 1952 74, 629. (b) Sugii, Y.; Shindo, H. J. Pharm. Soc. Japan 1934, 54, 829.

7.17. Kleiderer, E. C.; Adams, R. J. Am. Chem. Soc. 1933, 55, 4219.

7.18. Cornforth, Sir J.; Hugein, L. M.; Wilson, J. R. H. J. Chem. Soc. Perkins Trans. 1 1987, 871.

7.19. Costero, A. M.; Pitarch, M. J. Chem. Res. (M) 1994, 0761.

7.20. Waldeland, C. R.; Zartman, W.; Adkins, H. J. Am. Chem. Soc. 1933, 55, 4234.

7.21. Hofmeister, H.; Annen, K.; Laurent, H.; Wiechert, R. Angew. Chem. 1984, 96, 720.

7.22. Weiberth, F. J.; Hall S. S. J. Org. Chem. 1985, 50, 5310.

7.23. Silberrad, O.; Phillips, H. A. J. Chem. Soc. 1908, 474-476.

7.24. Hegelson, R. C.; Weisman, G. R.; Toner, J. L.; Tarnowski, T. L.; Chao, Y.; Mayer, J. M.; Cram, D. J. J. Am. Chem. Soc. 1979, 101, 4928-4941.

7.25. Newcomb, M.; Timko, J. M.; Walba, D. M.; Cram, D. J. J. Am. Chem. Soc. 1977, 99, 6404.

7.26. Reinhoudt, D. N.; Gray, R. T.; Smit, C. J.; Veenstra, Ms. I. Tetrahedron, 1976, 32, 1161.

8.2 Monks, A., Scudiero, D., Skehan, P., Shoemaker, R., Paull, K., Vistica, D., Hose, C., Langley, J., Cronise, P., Vaigro-Wolff, A., Gray-Goodrich, M., Campbell, H., Mayo, J., and Boyd, Michael. (1991) Feasibility of a high-flux anticancer drug screen using a diverse panel of cultured human tumor cell lines. J. Nat. Cancer Inst. 83:757-766.

8.4 Boyd, M. R.; The Future of New Drug Development; In Niederhuber, J. E. (ed.):Current Therapy in Oncology. Philadelphia, B. C. Decker, Inc., 1993, pp. 11-22.

8.5 Boyd, M. R. and Paull, K. D.; Some Practical Consideration and Applications of the NCI In Vitro Drug Discover Screen. Drug Dev. Res. 34: 91-109, 1995.

8.6 Boyd, M. R.; The NCI In Vitro Anticancer Drug Discovery Screen; Concept, Implementation and Operation 1985-1995. In: Teicher, B. A. (ed.): Cancer Drug Discovery and Development, Vol. 2; Drug Development; Preclinical Screening, Clinical Trial and Approval, Humana Press, 1997, pp. 23-43.

The following references, not specifically referred to in the text above, are also incorporated by reference.

Paull, K D, Shoemaker, R H, Hodes, L., Monks, A, Scudiero, D. A., Rubinstein, L., Plowman, J., and Boyd, M. R. (1989) Display and analysis of patterns of differential activity of drugs against human tumor cell lines: Development of mean graph and COMPARE algorithm. J. Nat. Cancer Inst. 81:1088-1092.

Weislow, O. S., R. Kiser, D. L. Fine, J. P. Bader, R. H. Shoemaker, and M. R. Boyd. (1989) New soluble formazan assay for HIV-1 cytopathic effects: application to high flux screening of synthetic and natural products for AIDS antiviral activity. J. Nat. Cancer Inst. 81:577-586.

(a) Fukuda, T.; Takanaka, S.; Takagi, M. J. Chem. Soc., Chem. Comm. 1990, 1028. (b) Griffin, J. H.; Dervan, P. B. J. Am. Chem. Soc. 1987, 109, 6840. (c) Basak, A.; Dugas, H. Tetrahedron Lett. 1986, 27, 3.

Schwarzenbach, G.; Gysling, H. Helv. chim. Acta 1949, 32, 1314.

Dai, W -M.; Fong, K. C.; Danjo, H.; Nishimoto, S -i.; Solow, M.; Mak, W. L.; Yeung, M. L. Bioorg. Med. Chem. Lett. 1996, 6, 1093.

(a) van Remoortere, F. P. Inorg. Chem. 1974, 13, 2826. (b) Beer, P. D. J. Chem. Soc., Chem. Comm. 1986, 1678.

Beato, M.; Chavez, S.; Truss, M. Steroids 1996, 61, 240.

White, A.; Ding, X.; vanderSpek, J. C.; Murphy, J. R.; Ringe, D. Nature 1998, 394, 502.

An, H.; Bradshaw, J. S.; Izatt, R. M.; Yan, Z. Chem. Rev. 1994, 94, 939.

Silvana, F.; Bernadou, J. Meunier, B. Bioconjugate Chem. 1997, 8, 222.

Bennett, C. B.; Snipe, J. R.; Resnick, M. A. Cancer Research 1997, 57, 1970.

Dai, W -M.; Fong, K. C. Tetrahedron Lett. 1995, 36, 5613.

Nicolaou, K. C.; Zuccarello, G.; Skokotas, G.; Maligres, P. U.S. Pat. No. 5,237,101, issued Aug. 17, 1993.

Izatt, R. M.; Bradshaw, J. S.; Nielson, S. A.; Lamb, J. D.; Christensen, J. J. Chem. Rev. 1985, 85, 271.

(a) Gösseringer, R.; Küister, E.; Galinier, A.; Deutscher, J.; Hillen, W. J Mol. Biol. 1997, 266, 665. (b) Burz, D. S.; Ackers, G. K. Biochemistry 1996, 35, 3341.

(a) Hendrixson, R. R.; Mack, M. P.; Palmer, R. A.; Ottolenghi, A.; Ghiradelli, R. G. Toxicol. and Appl. Pharmacol. 1978, 44, 263. (b) Gad, S. C.; Reilly, C.; Siino, K., Gavigan, F. A.; Witz, G. Drug and Chemical Toxicology 1985, 8, 451.

(a) calicheamicin γ₁^{I}: Zein, N.; Poncin, M.; Nilakantan, R.; Ellestad, G. A. Science 1989, 244, 697. (b) dynemicin A: Konishi, M.; Ohkuma, H.; Matsumoto, K.; Tsuno, T.; Kamei, H.; Miyaki, T.; Oki, T.; Kawaguchi, H.; Van Duyne, G. D.; Clardy, J. J. Antibiot. 1989, 42, 1449.

Grissom, J. W.; Gunawardena, G. U.; Klingberg, D.; Huang, D. Tetrahedron, 1996, 52, 6453.

(a) Bergman reaction: Bergman, R. G. Acc. Chem. Res. 1973, 6, 25. (b) radical-induced DNA damage: Dedon, P. C.; Goldberg, I. H. Chem. Res. Tox. 1992, 5, 311.

(a) Walker, S.; Landovitz, R.; Ding, W. -D.; Ellestad, G. A.; Kahne, D. Proc. Natl. Acad. Sci. USA 1992, 89, 4608. (b) Hangeland, J. J.; De Voss, J. J.; Heath, J. A.; Townsend, C. A.; Ding, W. -D.; Ashcroft, J. S.; Ellestad, G. A. J. Am. Chem. Soc. 1992, 114, 9200.

Nicolaou, K. C.; Zuccarello, G.; Ogawa, Y.; Schweiger, E. J.; Kumazawa, T. J. Am. Chem. Soc. 1988, 110, 4866.

(a) Semmelhack, M. F.; Gallagher J. J.; Minami, T.; Date, T. J. Am. Chem. Soc. 1993, 115, 11618. (b) Nicolaou, K. C.; Dai, W. -M.; Tsay, S. C.; Estevez, V. A.; Wrasidlo, W. Science 1992, 256, 1172. (c) Audrain, H. Skydstrup, T.; Ulibarri, G.; Grierson, D. S. Synlett 1993, 20. (d) Takahashi, T; Tanaka, H.; Yamada, H.; Matsumoto, T.; Sugiura, Y. Angew. Chem. Int. Ed. Engl. 1996, 35, 1835.

Wender, P. A.; Zercher, C. K.; Beckham, S.; Haubold, E. -M. J. Org. Chem. 1993, 58, 5667.

Banfi, L.; Basso, A.; Guanti, G. Tetrahedron 1997, 53, 3249.

Myers, A. G.; Dragovich, P. S. J. Am. Chem. Soc. 1992, 114, 5859.

Maier, M. E. Synlett 1995, 13.

Hay, M. P.; Wilson, W. R.; Denny, W. A. Bioorg. Med. Chem. Lett. 1995, 5, 2829.

König, B.; Rüitters, H. Tetrahedron Lett. 1994, 35, 3501.

König, B.; Pitsch, W.; Thondorf, I. J. Org. Chem. 1996, 61, 4258.

Warner, B. J.; Millar, S. P.; Broene, R. D.; Buchwald, S. L. Science 1995, 269, 814.

Reinhoudt, D. N.; Gray, R. T.; Smit, C. J.; Veenstra, Ms. I. Tetrahedron 1976, 32, 1161.

Pedersen, C. J. J. Am. Chem. Soc. 1967, 89, 7017.

McPhee, M. M.; Kerwin, S. M. J. Org. Chem. 1996, 61, 9385.

Myers, A. G.; Dragovich, P. S. J. Am. Chem. Soc. 1989, 111, 9130.

Stephens, R. D.; Castro, C. E. J. Org. Chem. 1963, 28, 3313.

March, J.; Advanced Organic Chemistry 4th Ed., John Wiley and Sons, New York, 1992, 714.

Beau, J. -M.; Crévisy, C. Tetrahedron Lett. 1991, 32, 3171.

Audrain, H.; Skydstrup, T.; Ulibarri, G.; Ridu, C.; Chiaroni, A.; Grierson, D. S. Tetrahedron 1994, 50, 1469.

Nishikawa, T.; Ino, A.; Isobe, M. Tetrahedron 1994, 50, 1449.

Shair, M. D.; Yoon, T.; Danishefsky, S. J. J. Org. Chem. 1994, 59, 3755.

Paquette, L. A. Org React. 1977, 25, 1.

McMurry, J. E.; Lectka, T.; Rico, J. G. J. Org. Chem. 1989, 54, 3748.

Jones, G. B.; Huber, R. S.; Mathews, J. E. J. Chem. Soc., Chem. Comm. 1995, 1791.

Huber, R. S.; Jones, G. B. Tetrahedron Lett. 1994, 35, 2655.

Ferguson, M. D.; Butcher, J. A. Jr. Tetrahedron Lett. 1993, 34, 3719.

Brandsma, L. Preparative Acetylenic Chemistry, Elsevier, Amsterdam 1971, 30.

Noro, M.; Masuda, T.; Ichimura, A. S.; Koga, N.; Iwamura, H. J. Am. Chem. Soc. 1994, 116, 6177. (b) Padwa, A.; Gareau, Y.; Xu, S. L. Tetrahedron Lett. 1991, 32, 983.

Wang, P.; Adams, J. J. Am. Chem. Soc. 1994, 116, 3296.

Charette, A. B.; Marcois, J. -F.; Bélanger-Gariépy, F. J. Am. Chem. Soc. 1996, 118, 6792.

Jones, G. B.; Wright, J. M.; Rush, T.; Plourde, G. W., II; Kelton, T. F.; Mathews, J. E.; Huber, R. S.; Davidson, J. P. J. Org. Chem. 1997, 62, 9379.

Lienne, M.; Gareil, P.; Rosset, R.; Husson, J. F.; Emmelin, M.; Neff, B. J. Chromatography 1987, 395, 255.

Semmelhack, M. F.; Neu, T.; Foubelo, F. J. Org. Chem. 1994, 59, 5038.

Taskinen, E.; Nummelin, K. J. Org. Chem. 1985, 50, 4844.

Wender, P. A.; Tebbe, M. J. Tetrahedron, 1994, 50, 1419.

(a) Christner, D. F.; Frank, B. L.; Kozarich, J. W.; Stubbe, J.; Golik, J.; Doyle, T. W.; Rosenberg, I. E.; Krishnan, B. J. Am. Chem. Soc. 1992, 114, 8763. (b) Kishikawa, H.; Jiang, Y. P.; Goodisman, J. P.; Dabrowski, J. C. J. Am. Chem. Soc. 1991, 113, 5434.

Denny, W. A. Curr. Pharm. Design 1996, 2, 281.

Taylor, P.; Insel, P. A. in Principles of Drug Action, 3rd Ed., Pratt, W. B.; Taylor, P., Eds., Churchill Livingstone, New York, 1990, 103.

(a) Kelly, T. R.; Bowyer, M. C.; Bhasaker, K. V.; Bebbington, D.; Garcia, A.; Lang, F.; Kim, M. H.; Jette, M. P. J. Am. Chem. Soc. 1994, 116, 3657. (b) Beer, P. D.; Rothin, A. S. J. Chem. Soc., Chem. Comm. 1988, 52. (c) Rebek, J. Jr.; Wattley, R. V. J. Am. Chem. Soc. 1980, 102, 4853.

Rebek, J. Jr.; Costello, T.; Marshall, L.; Wattley, R.; Gadwood, R. C.; Onan, K. J. Am. Chem. Soc. 1985, 107, 7481.

Snyder, J. P. J. Am. Chem. Soc. 1989, 111, 7630.

Chen, X. -M.; Luo, G. -B.; Tong, M. -L.; Zhou, Z. -Y. Acta Crystalloghaphica Section C 1996, 1727.

(a) Schake, A. R.; Schmitt, E. A.; Conti, A. J.; Streib, W. E.; Huffman, J. C.; Hendrickson, D. N.; Christou, G. Inorg. Chem. 1991, 30, 3192. (b) Goldberg, D. P.; Caneschi, A.; Lippard, S. J. J. Am. Chem. 1993, 115, 9299.

Durairaj, K. Current Science 1994, 66, 833.

Xi, M.; Bent, B. E. J. Am. Chem. Soc. 1993, 115, 7426.

Skoog, D. A.; Leary, J. L. Principles of Instrumental Analysis, 4th Ed., Harcourt Brace Jovanovich, Fort Worth, 1992, 138.

Gruter, G. -J.; Akkerman, O. S.; Bickelhaupt, F. J. Org. Chem. 1994, 59, 4473.

Parker, K. A.; Koziski, K. A. J. Org. Chem. 1987, 52, 674.

Lipshutz, B. H.; James, B.; Vance, S.; Carrico, I. Tetrahedron Lett. 1997, 38, 753.

Claesson, A.; Sahlberg, C. Tetrahedron, 1982, 38, 363.

Rossi, R.; Carpita, A.; Lippolis, V.; Benetti, M. Gazz. Chim. Ital. 1990, 120, 783.

Kasturi, T.; Mandal, A. B.; Sumana, B. G.; Rajasekhar, B. Tetrahedron 1993, 49, 2751.

Schlosser, M. Organometallics in Synthesis, A Manual, John Wiley & Sons, New York, 1994, 75.

Hofmeister, H.; Annen, K.; Laurent, H.; Wiechert, R. Angew. Chem. 1984, 96, 720.

Meijer, J.; Vermeer, P. Rec. Trav. Chim. 1974, 93, 183.

Weiberth, F. J.; Hall, S. S. J. Org. Chem. 1985, 50, 5310.

Wang, K. K. Chem. Rev. 1996, 96, 207.

Takada, T.; Arisawa, M.; Gyoten, M.; Hamada, R.; Tohma, H.; Kita, Y. J. Org. Chem. 1998, 63, 7698.

Adams, R.; Komblum, N. J. Am. Chem. Soc. 1941, 63, 188.

Bell, T. W.; Lein, G. W.; Nakamura, H.; Cram, D. J. J. Org. Chem. 1983, 48, 4782.

Costero, A. M.; Pitarch, M. J. Chem. Res. (M) 1994, 0761.

(a) for an example of the preparation and use of organocerium reagents, see: Imamoto, T.; Sugiura, Y.; Takiyama, N. Tetrahedron Lett. 1984, 25, 4233. (b) for an example of a non-basic organoaluminum reagent, see: Tolstikov, G. A.; Prokhorova, N. A.; Spivak, A. Y.; Khalikov, L. M.; Sultanmuratova, V. R. Zh. Org. Khim. 1991, 27, 2101.

Kang, J.; Cho, W.; Lee, W. K. J. Org. Chem. 1984,49, 1838.

Cooper, T. A. J. Am. Chem. Soc. 1973, 95, 4158 , and references therein.

Huynh, C.; Linstrumelle, G. Tetrahedron 1988, 6337.

Stephen, J.; Osborne, M. P.; Spencer, A. J.; Warley, A. Scanning Microsc. 1990, 4, 781.

Zs.-Nagy, I. Scanning Microsc. 1989, 3, 473.

Roomans, G.; Von Euller, A. Cell Biol. Int. 1996, 20, 103.

Cameron, I. L.; Smith, N. K. R. Magnesium 1989, 8, 31.

Smith, P.; Rhodes, N. P.; Shortland, A. P.; Fraser, S. P.; Djamgoz, M. B. A.; Ke, Y.; Foster, C. S. FEBS Lett. 1998, 423, 19.

Besson, P.; Gore, J.; Vincent, E.; Hoinard, C.; Bougnoux, P. Biochem. Pharmacol. 1996, 51, 1153.

What is claimed is:

1. A compound of general structure I: 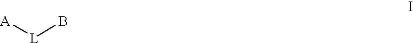
where A comprises an alkali metal ion binding moiety, B comprises a DNA-interactive moiety capable of covalent modification of DNA, and L comprises a linking group.

2. The compound of claim 1, wherein the alkali metal ion binding moiety comprises a crown ether, a cryptand, a sepulchrate, a spherand, a calixarane, a cyclen, a polyether antibiotic, a cyclic peptide antibiotic, or a podand.

3. The compound of claim 1, wherein the alkali metal ion binding moiety comprises a crown ether or a podand.

4. The compound of claim 1, wherein the DNA-interactive moiety comprises a propargylic sulfone, an enediyne, an aza-enediyne, an eneynallenes, an aza-eneynallene, a cyclopropylpyrroloindole, a pyrrolobenzodiazepines, a nitrogen mustard, a sulfur mustard, an epoxide, an aziridine, a nitroso compound, an iron-EDTA complex, a sulfonate ester, an alkyl halide, an ortho-quinone-generating moiety, a photo-activated DNA cleavage agent, an azide, a benzophenone, a quinobenzoxazine, a fluoroquinolone, a Rh-complex, a Ru-complex, a Cu-complex, a Co-complex, a bleomycin, a bleomycin analog, a porphyrins, a porphyrin analog, and a metal salen complex.

5. The compound of claim 1, wherein the DNA-interactive moiety comprises a propargylic sulfone, an enediyne or a sulfonate ester.

6. The compound of claim 1, wherein the alkali metal ion binding moiety comprises a crown ether, the DNA interactive moiety comprises a bis-propargylic sulfone, and the linking group comprises an alkane.

7. The compound of claim 1, wherein the alkali metal ion binding moiety comprises a crown ether, the DNA interactive moiety comprises an enediyne, and the linking group comprises an alkane.

8. The compound of claim 1, wherein the alkali metal ion binding moiety comprises a crown ether, the DNA interactive moiety comprises a biphenyl enediyne, and the linking group comprise an alkane.

9. The compound of claim 1, having the general structure: 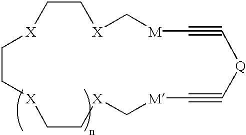
where each X is independently O, N, or S; n=1 to 4; M and M′ are CH₂ or together form a biphenyl ring; R is CH₂—S—CH₂; CH₂—SO₂—CH₂; or CH₂═CH₂.

10. The compound of claim 1, having the general structure: 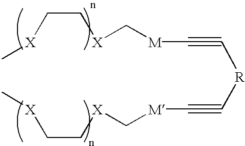
where X is independently O, N, or S; n=1 to 4; M and M′ are CH₂ or together form a biphenyl,; R is CH₂—S—CH₂; CH₂—SO₂—CH₂; or CH₂═CH₂.

11. The compound of claim 1, having the general structure: 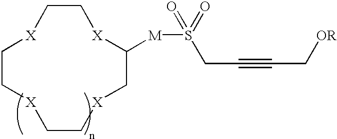
where X is independently O, N, or S; n=1 to 4; and M is alkyl, ester, or amide; and R is hydrogen or another C group which together forms a dimeric structure.

12. The compound of claim 1, having the general structure: 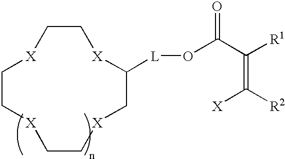
where X is independently O, N, or S; n=1 to 4; and L is a linking group, R¹ is H, R² is CH₂═CH₂—C≡C—CH₂—OH or R¹ and R² together form a compound having the structure: 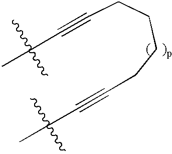
where p=1 to 4, and where X is a halogen or OTf.

13. The compound of claim 1, wherein the compound is capable of producing a diradical intermediate at physiological conditions.

14. The compound of claim 1, wherein the compound binds to alkali metals.

15. The compound of claim 1, wherein the compound binds to alkali metals, and wherein the compound is configured to effect nucleic acid cleavage.

16. The compound of claim 1, having the general structure II: 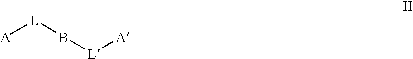
where A and A′ comprise the same or different alkali metal ion binding moiety, B comprises a DNA-interactive moiety capable of covalent modification of DNA, and L and L′ are comprise linking groups.

17. The compound of claim 1, having the general structure III: 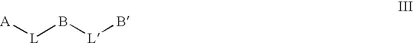
where A comprises an alkali metal ion binding moiety, B and B′ comprise DNA-interactive moieties capable of covalent modification of DNA, and L and L′ comprise linking groups.

18. The compound of claim 1 having the general structure IV 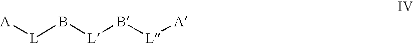
where A and A′ comprise the same or different alkali metal ion binding moiety, B and B′ comprise DNA-interactive moieties capable of covalent modification of DNA, and L, L′, and L″ comprise linking groups.

19. The compound of claim 1 having the general structure V: 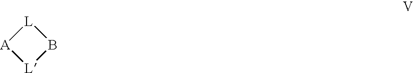
were A comprises an alkali metal ion binding moiety, B comprises a DNA-interactive moiety capable of covalent modification of DNA, and L and L′ comprise linking groups.

20. The compound of claim 1 having the general structure VI 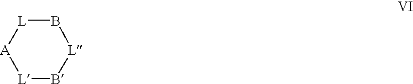
where A comprises an alkali metal ion binding moiety, B and B′ comprise DNA-interactive moieties capable of covalent modification of DNA, and L and L′ comprise linking groups.
